(19) United States
(12) Patent Application Publication
(10) Pub. No.: US 2024/0084261 A1
(43) Pub. Date: Mar. 14, 2024

(54) Cell Clusters Comprising Stem and Islet Cells, Methods of Making, and Treatment of Diabetes Mellitus Therewith

(71) Applicant: SYMBIOCELLTECH, LLC, Salt Lake City, UT (US)

(72) Inventors: Christof Westenfelder, Salt Lake City, UT (US); Anna Maddock Gooch, Salt Lake City, UT (US)

(21) Appl. No.: 18/263,934

(22) PCT Filed: Feb. 3, 2022
(86) PCT No.: PCT/US2022/015066

§ 371 Date: Aug. 2, 2023

Related U.S. Application Data
(60) Provisional application No. 63/145,380, filed on Feb. 3, 2021.

(51) Int. Cl. C12N 5/071; A61P 3/10

(52) U.S. Cl. CPC C12N 5/0677; A61P 3/10; C12N 2502/1358; C12N 2502/1382

(57) ABSTRACT

Provided are compositions and methods of treating impaired glucose tolerance which substantially increase the islet donor pool and safety as well as remove the need for anti-rejections drugs. Described are methods of making cell clusters comprising: expanding pancreatic islet cells; and forming cell clusters comprising: the expanded islet cells and mesenchymal/adipose stem cells. Expansion of the islet cells may include=>five population doublings. The islet cells may be primary islet cells obtained from an adult, non-diabetic donor having a North America Islet Donor Score (NAIDS) of <80. Described are cell clusters produced by the methods. Additionally, methods of treatment by intraperitoneal administration of islet-sized cell clusters composed of high numbers of mesenchymal stem cells and expanded pancreatic islet cells, durably and reversibly treats, without hypoglycemia, both streptozotocin-induced and spontaneous Type 1 Diabetes Mellitus, Type 2 Diabetes Mellitus, and other types of insulin-dependent diabetes mellitus, or impaired glucose tolerance.

### CROSS-REFERENCE TO RELATED APPLICATIONS

This application is a national phase entry under 35 U.S.C. § 371 of International Patent Application PCT/US2022/015066, filed Feb. 3, 2022, designating the United States of America and published in English as International Patent Publication WO 2022/169943 on Aug. 11, 2022, which claims the benefit under Article 8 of the Patent Cooperation Treaty to U.S. Patent Application Ser. No. 63/145,380, filed Feb. 3, 2021, the entireties of which are hereby incorporated by reference.

### TECHNICAL FIELD

The application relates to the field of biotechnology, medicine, and cell culture. It specifically relates to, e.g., methods of producing compositions of and comprising cell clusters (also identified as “Neo-Islets” or “NIs”) that include stem cells and pancreatic islet cells (ICs). It also relates to the utilization of cell clusters comprising stem cells and pancreatic islet cells for treatment of, for example, insulin-dependent diabetes mellitus, noninsulin-dependent diabetes mellitus, or impaired glucose tolerance.

### BACKGROUND

Insulin-producing β-Cells, when isolated from a donor pancreas, generally proliferate very poorly ex vivo, i.e., not sufficiently to generate adequate cell numbers for the treatment of insulin-dependent diabetes mellitus. Current technologies and many preclinical therapies designed to overcome this shortage and provide diabetic patients with a long-lasting, physiologically released insulin replacement therapy (islet and pancreas transplants; precursor cell-derived therapies, etc.) are hampered both by the shortage of donor cells and the need to suppress the patient's immune system, leading to a new set of adverse effects for the patient, such as opportunistic infections and malignancies. The great shortage of suitable pancreas donors combined with the need for repeated islet transplants, requiring up to five donors each, continues to prevent the general availability of these expensive therapies. Micro- and macro-encapsulation systems of insulin-producing cells are tested to facilitate immune isolation and overcome this problem. However, the utilized encapsulation materials represent foreign bodies and can induce a foreign body reaction that will result in the failure of the therapy or require use of anti-rejection drugs if the encapsulation device is open.

### BRIEF SUMMARY

Described herein are methods of making cell clusters, the method comprising: expanding pancreatic islet cells; and forming cell clusters comprising: the expanded pancreatic islet cells; and mesenchymal stem cells and/or adipose stem cells.

In embodiments, expansion of the pancreatic islet cells includes at least five population doublings before forming the cell clusters.

In embodiments, the pancreatic islet cells are primary pancreatic islet cells obtained from an adult donor; and wherein the adult donor's islets had a North America Islet Donor Score (NAIDS) of less than 80.

Further described herein are cell clusters produced by the method described herein.

Also described are methods of treating a subject, the methods comprising: providing to the subject the cell clusters described herein. Additionally, described are methods of treating a subject suffering from Type 1 Diabetes Mellitus or Type 2 Diabetes Mellitus, by, e.g., providing to the subject cell clusters as described herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: depicts a schematic overview of cell cluster formation and their use in treatment of insulin-dependent diabetes mellitus or noninsulin-dependent diabetes mellitus.

FIG. 2: Outgrowth and Epithelial to Mesenchymal transition of cultured pancreatic islet cells. All images are at 10× magnification. Panel A: Whole islets freshly isolated from the transgenic C57Bl/6 ins1gfp mouse, wherein the Green Fluorescent Protein gene (gfp) is under the control of the Insulin 1 (ins1) gene promoter.[1] Islet beta cells are rendered green. Panel B: Ins1gfp+ whole islets after 6 days of culture. Significant insulin gene expression is still apparent (green cells), and cells are outgrowing from the islets and proliferating. In these outgrown cells, insulin gene expression will be downregulated and the cells will no longer be green. Panel C: Dissociated ins1gfp+ mouse Islet cells cultured for 1 day, and fixed and analyzed by immunocytochemistry for insulin protein, using a guinea-pig anti-insulin antibody, and a cy³-conjugated anti-guinea pig antibody to visualize insulin protein (red). The image reveals that while there are very few gfp+ cells, approximately half the cells contain insulin, yet are not green. This indicates that the beta cells attach and proliferate as they lose the expression of the insulin gene due to Epithelial Mesenchymal transition. Panel D: Dissociated Ins1gfp+ pancreatic islet cells cultured 2 days in the presence of EdU. Cells were fixed and stained with Hoechst33342 (nuclei, blue, to identify non-mitogenic nuclei) and for EdU (red, to identify mitogenic nuclei). Cells that are not dividing are bright green and have a round, epithelial morphology, while cells that are dividing (red nuclei) are taking on an elongated, mesenchymal appearance, and are only faintly green, indicating the down-regulation of insulin gene expression, and illustrating their Epithelial Mesenchymal transition.

FIG. 3: Comparative, passage (P) dependent gene expression profiles of NI starting materials. Gene expression profiles (Log 10RQ) of mouse, dog and human cultured pancreatic islet cells (left) and M/ASCs (right) at passages 1, 2 and 3 (P1, P2, and P3, respectively). All gene expression profiles for both cell types were normalized to those of species-specific, freshly isolated islets. Overall, in mice, dogs and humans, gene expression profiles of M/ASCs differ from those of passaged pancreatic islet cells, and passaging of pancreatic islet cells progressively decreases the expression of islet cell associated genes. Data: mean with 95% CI, representative of six independent experiments. □, expressed in hASCs, but not in human pancreatic islet cells, preventing respective normalization; *, not expressed.

FIGS. 4A-4D. Mouse (FIG. 4A) and canine (FIG. 4B) ASC phenotyping. FIGS. 4A and 4B, left upper panels: bright field images of plastic adherent, confluent ASC cultures; Right upper panels: Osteogenic differentiation (calcium staining with Alizarin Red); Left lower panels: adipogenic differentiation (Oil Red-O staining); Right lower panels: Chondrogenic differentiation (Alcian blue staining) Red scale bars=50 μm. FIG. 4C: IDO-1 gene expression of canine ASCs exposed to IFNγ normalized to that of unexposed cASCs (mean±SEM of 4 independent experiments). IDO-1 gene expression is stimulated 3.4-fold when canine ASCs are exposed to IFNγ. FIG. 4D: Cultured mouse and dog ASCs were examined by FACS for their positive expression of CD44, and negative expression of CD45, CD34 and I-A[b] and DLA-DR transplant antigens. While all M/ASCs are characterized by plastic adherence and ability to undergo trilineage differentiation, not all non-human M/ASCs express the same set of cell surface epitopes as those from humans, and while most express CD44, expression of CD90 is variable. Canine M/ASC expression of CD90 is variable.

FIG. 5: cell cluster formation and imaging. FIG. 5 depicts images and a schematic of mouse cells undergoing cell cluster formation, wherein 1) green fluorescent protein positive (gfp+) mouse MSCs are culture expanded; 2) Mouse pancreatic islet cells are culture expanded; 3) the cells are co-cultured in ultra-low-adhesion plates and readily form cell clusters. The cell clusters can subsequently be cultured in redifferentiation medium (RDM).

FIG. 6: LEFT, MIDDLE and RIGHT PANELS: images (63× magnifications) of Murine (left), Canine (middle) and Human (right) cell clusters; ASCs (green), pancreatic islet cells (red) and nuclei (blue). Morphology and cell composition do not differ significantly among murine, canine and human cell clusters.

FIGS. 7A and 7B. Percent of Cell Tracker Green stained dog ASCs and unstained dog islet cells in cNIs prior to and post formation. FIG. 7A: Representative FACS scatter plots (x=forward scatter; y=fluorescence) showing the percent of green and unstained (i) ASCs alone (far left panel), (ii) ICs alone (left middle panel) (iii) cells at initiation of co-culture (right middle panel), (iv) dissociated NIs obtained from the co-culture in (iii; far right panel) 24 hrs. post-co-culture collected and dissociated to single cell preparation. 96.9% of the ASCs were effectively stained green prior to co-culture and only 0.1% of the unstained ICs are autofluorescent. Upon initiation of co-culture, approximately 47% of the cells are ASC and 53% are ICs, and after co-culture, within the NIs, approximately 51% of the cells are ASCs and 49% are ICs. FIG. 7B: Bar graph of the percent of ASCs and ICs in NIs 24 hrs. post-coculture (mean±SEM) of n=12 independent repetitions of the experiment conducted in FIG. 7A, indicating that consistently, NIs are composed of approximately 50% ICs and 50% ASCs. Differences between bars are not statistically significant.

FIGS. 8A-8C: Gene Expression Profiles of Mouse, Dog, and Human cell clusters. All data are normalized to 2 housekeeping genes, β-actin and β2 microglobulin. FIG. 8A: Gene expression profile of islet associated genes in mouse (top) and dog (bottom) cell clusters. Gene expression profiles were obtained from redifferentiated mouse cell clusters (top left), freshly isolated mouse islets (top right), and freshly formed mouse cell clusters (normalization is to mouse islets) on the following 14 islet associated genes: insulin 1 (ins1), insulin 2 (ins2), glucagon (gcg), somatostatin (sst), pancreatic duodenal homeobox-1 (pdx1), Insulin transcription factor mafA (mafa), nk6 homeobox 1 (nkx6.1), pancreatic polypeptide (ppy), glut-1, glut-2, ucn3, kcj11, sur2 and glp1 receptor (glp1r). Gene expression profiles were obtained from redifferentiated dog cell clusters (bottom left), freshly isolated dog islets (bottom right), and freshly formed dog cell clusters (dog normalization) on the following 6 islet associated genes: insulin (ins), gcg, sst, nkx6.1, sur1, and glp1r. Both freshly formed mouse and dog cell clusters express low levels of all tested islet associated genes, and have the capacity to undergo redifferentiation resulting in higher levels of these genes. FIG. 8B: Top: Gene expression profiles for insulin 1 (ins1), insulin 2 (ins2), glucagon (gcg), somatostatin (sst), pancreatic polypeptide (ppy), pancreatic duodenal homeobox-1 (pdx1), Insulin transcription factor mafA (mafa), glucose transporter 2 (glut-2), vascular endothelial growth factor α (vegf-α) and stromal cell derived factor 1 (cxcl-12) were obtained from freshly formed mouse cell clusters generated from either P1 mouse pancreatic islet cells and P5 mouse MSCs (left), or P2 mouse pancreatic islet cells and P5 mouse MSCs (right), and normalized to freshly isolated mouse islets. Middle: gene expression profiles of islet cell associated genes, insulin (ins), gcg, pdx1 and sulfonylurea receptor 1 (sur1), as well as ASC associated genes vegf-α, cxcl12, and transforming growth factor 131 (tgfβ-1) in freshly formed canine cell clusters produced from either P1 dog pancreatic islet cells and P2 dog ASCs (left) or P2 dog pancreatic islet cells and P2 dog ASCs (right) and normalized to freshly isolated dog islets. Bottom: Gene expression profile for ins, gcg, sst, ppy, pdx1, mafa, nk6 homeobox 1 (nkx6.1), urocortin 3 (ucn3), sur1, vegf-α, cxcl12, tgfβ-1, and igf-1 in freshly formed human cell clusters generated from either P1 human pancreatic islet cells and P3 human ASCs (left) or P2 human pancreatic islet cells and P3 human ASCs (right) normalized to freshly isolated human islets. This panel demonstrates that across species (murine, canine, human), (a) cell clusters made from dedifferentiated, passaged pancreatic islet cells express low levels of islet cell genes, and (b) islet cell gene expression decreases with passaging. FIG. 8C: Glucose Stimulated Insulin Secretion (GSIS) by 50 freshly formed C57Bl/6 mouse cell clusters comprising dedifferentiated P1 pancreatic islet cells and P5 MSCs (cross hatched bars) vs. 50 freshly isolated C57Bl/6 mouse islets (open bars). Experiments were performed in duplicate. Cell clusters release approximately 1% of the insulin that freshly isolated islets do in response to exposure to 25 mM glucose for 60 minutes (˜0.5 ng vs. ˜50 ng Insulin). This parallels the decrease in insulin gene expression over passages seen in Panel B.

FIG. 9. Allogeneic NI-treatment established euglycemia in spontaneously diabetic NOD mice. Blood glucose levels (mean±SEM) of NOD mice normalized with sub-cutaneous (s.c.) insulin-releasing Linbit pellets (Linbits) (Day 0), then infused i.p. on Day 20 post Linbit therapy with 2×10⁵ C57Bl/6 NI/kg (n=6; open bars) or vehicle (n=6; black bars). While vehicle treated mouse blood glucose levels increased when Linbits expired (approximately Day 35), euglycemia was maintained long term in NI treated mice, implying IC redifferentiation into insulin producing cells and NI-mediated immune protection from allo- and autoimmune attacks. Normal blood glucose level, hashed line. *, P<0.05 vs. vehicle treated group.

FIGS. 10A-10C. Blood glucose levels of NI and cluster treated, STZ diabetic C57Bl/6 mice and in vivo redifferentiation of ICs into endocrine cells contained in the NIs. FIG. 10A: Blood glucose levels over time are shown in groups of STZ-diabetic mice all treated i.p. on Day 7 with (i) vehicle, (ii) 2×10⁵ ASC clusters/kg b.wt., (iii) 2×10⁵ IC clusters/kg b.wt. or (iv) 2×10⁵ NIs/kg b.wt. *, P<0.05 vs. vehicle-treated group. ★, P<0.05 vs. ASC-cluster treated group. FIG. 10B: Left, Fluorescence image (green, eGFP+ cells) of a representative omentum from an NI-treated, euglycemic mouse 21 weeks post NI injection (scale bar=200 μm). Right, omental gene expression (mean±SEM) normalized to that of NIs prior to administration, demonstrating NI engraftment, and significant endocrine redifferentiation. FIG. 10C: Ins1 and Ins2 expression profiles (mean±SEM) from whole pancreata of ASC-cluster, IC-cluster, and NI treated vs. vehicle-treated diabetic mice normalized to those of non-diabetic mice. Since pancreatic insulin gene expression levels were similarly decreased in all treatment groups vs. those of hyperglycemic, vehicle-treated mice, it follows that the blood glucose control seen in NI-treated mice was achieved by insulin secretion from omental NIs.

FIG. 11: Morphology and viability of C57Bl/6 IC- and ASC clusters used to treat mice in FIGS. 10A-10C. Fluorescence images of P1 IC-only (left) and P1 ASC-only (right) clusters post formation and stained for viability with PI (red) and FDA (green, see Methods). ASC-only and IC-only clusters are >95% viable prior to i.p. injection. Scale bar (red)=150 μm.

FIG. 12: Blood Glucose Profiles and Dose Finding Study in NOD/SCID mice treated i.p. three weeks after STZ-Induced Hyperglycemia, with vehicle or canine cell clusters. Both the 2×10⁵ (black bars) and 8×10⁴ cell clusters/kg bw (cross hatched bars) doses reduce blood glucose levels long term compared with vehicle treatment (open bars). However, 2×10⁵ cell clusters/kg body weight (“bw”) is a more effective dose.

FIG. 13: Reversal of Euglycemia by removal of Canine cell clusters. Treatment i.p. of STZ diabetic NOD/SCID mice with canine cell clusters (black bars) causes sustained euglycemia compared to vehicle-treated animals (open bars), while removal of canine cell clusters from such treated animals results in return of hyperglycemia.

FIGS. 14A and 14B. I.P. administered syn- and xenogeneic NIs normalize blood glucose levels of STZ-diabetic mice. FIG. 14A: Blood glucose levels over time of STZ-diabetic C57Bl/6 mice treated i.p. with 2×10⁵ syngeneic NIs/kg b.wt. (black bars, N=6) or vehicle (open bars, N=6). FIG. 14B: Blood glucose levels of STZ-diabetic NOD/SCID mice treated with either 2×10⁵ canine NI/kg b.wt. (black bars, N=5) or vehicle (open bars, N=5). In both FIGS. 14A and 14B, blood glucose levels were first normalized on Day zero with Linbit pellets prior to NI administration. While vehicle-treated mice become hyperglycemic once insulin is depleted from the Linbits (30-40 days post implantation), NI treated mice (syn- and xenogeneic) remain normoglycemic, indicating NIs control blood glucose levels long term. *, P<0.05 vs. vehicle-treated groups.

FIG. 15: Kaplan-Meier survival plots of STZ-diabetic NOD/SCID mice treated early after the development of diabetes with canine cell clusters or vehicle. Diabetic animals treated with either the 2×10⁵ (squares) or 8×10⁴ (circles) cell clusters/kg bw dose survive significantly longer than vehicle-treated (triangle), or, surprisingly, non-diabetic control (diamonds) animals.

FIG. 16: Intraperitoneal Glucose Tolerance Test (IP GTTs) and Canine Insulin ELISA of cell cluster-treated, STZ-diabetic NOD/SCID mice. Top: IP GTTs Experimental Protocol. Bottom Left: IP GTTs of 2×10⁵ cell clusters/kg bw-treated (squares, n=5) vs. vehicle-treated NOD/SCID mice (circles, n=3). IP GTTs are normal in 2×10⁵ cell clusters/kg bw-treated, STZ-diabetic NOD/SCID mice, while blood glucose levels of vehicle-treated animals remain significantly elevated. Bottom Right: Canine-specific insulin ELISA conducted on duplicate samples of sera from vehicle (left bar, n=3) and canine cell cluster-treated (middle, cross-hatched bar, n=5), STZ-diabetic NOD/SCID mice that had been collected during the glucose tolerance tests, as well as sera from non-diabetic C57Bl/6 mice (middle black bar, n=2, negative control for ELISA specificity) and a healthy dog (open bar, positive control for ELISA specificity). In canine cell cluster-treated, but not vehicle-treated mice, a rise in blood glucose is accompanied by release of canine insulin, indicating that insulin release from the canine cell clusters is responsible for the normal IP GTTs.

FIG. 17: Insulitis remains in NI-treated NOD mice. Representative image (40×) of a Hematoxylin Eosin stained pancreatic section from a euglycemic, NI-treated NOD mouse 11 weeks post treatment demonstrating the presence of persistent, high-grade insulitis (black circle). Scale bar (white)=200 μm.

FIGS. 18A-18C. Omental NI engraftment, survival, and insulin expression in NOD mice. FIG. 18A: Bio-fluorescence in vivo imaging of a NOD mouse treated 10 weeks previously with DiR labeled, eGFP+ NIs demonstrates their location in the upper abdomen. FIG. 18B: eGFP+C57Bl/6 mouse NIs given i.p. remained engrafted in the omentum and maintained euglycemia in spontaneously diabetic NOD mice at 11 weeks post treatment (see FIG. 9). Left image (10×): representative omentum of a NOD mouse treated with C57Bl/6 eGFP+ NIs (green; see red arrows). This image demonstrates that the NIs homed to and engrafted in the omentum, and indicates there is no rejection of the NIs. Right image (10×): enlarged image of a single, engrafted NI. Its location, close to capillaries (yellow arrow) is shown. FIG. 18C: Left panel, Main image: Sections of the omentum (10× image) depicted in FIG. 18B stained by immunohistochemistry for DNA (Dapi, blue), and insulin protein (red). Insulin protein was clearly detected. Inset, negative control in which the primary, anti-insulin antibody was omitted. Right panel, Main image: Sections of the omentum (10×) of a vehicle treated, diabetic NOD mouse stained for DNA (blue), and insulin protein (red). Inset: 40× magnification of the same section (scale bar=10 μm). No insulin was detected at either magnification. These images demonstrate the omental location and insulin synthesis by engrafted NIs. Scale bars=100 μm unless otherwise indicated.

FIG. 19: Blood Glucose Levels of canine cell cluster-treated STZ-diabetic NOD/SCID mice. Animals were treated i.p. with cell clusters 3 months post onset of diabetes and followed Long-term Animals with established diabetes exhibit normoglycemia following treatment with canine cell clusters (black bars), while those treated with vehicle (open bars) remain hyperglycemic after insulin release by Linbits expires on ˜Day 36. This demonstrates that cell clusters are effective in establishing euglycemia in remote onset diabetes.

FIG. 20: Blood glucose levels of autoimmune T1DM NOD mice treated with allogeneic cell clusters. Spontaneously diabetic female NOD mice were treated with slow-release insulin pellets (Linbits, s.c.) to control hyperglycemia. On Day 20 post Linbit therapy, mice were treated with allogeneic cell clusters derived from C57Bl/6 mice (generated from P2 pancreatic islet cells and P5 gfp+ MSCs; n=5; black bars) or vehicle (n=3; open bars). These data clearly demonstrate that euglycemia is maintained as a consequence of cell cluster induced immune isolation against both allo- and autoimmune attacks.

FIG. 21: cell clusters do not induce hypoglycemia in non-diabetic mice. Top Panel: 2×10⁵ cell clusters/kg bw derived from C57Bl/6 mice were administered i.p. to non-diabetic C57Bl/6 mice on Day 0. Treated animals were followed for up to 12 weeks. Blood glucose levels were assessed weekly. No hypoglycemia was observed at any time point, demonstrating physiologic insulin release by mouse cell clusters. Cell clusters remain engrafted and were not rejected. Bottom Panel: Blood glucose levels of NOD/SCID mice treated i.p. with either (a) 2×10⁵ freshly formed DiR labeled dog cell clusters (P2 dog pancreatic islet cells+P4 dog ASCs; grey line; n=6) or (b) 0.5 ml serum free DMEM-F12 (Control; black line; n=3). Cell clusters remain engrafted (see FIG. 18A). No hypoglycemia was observed at any time point. These data further demonstrate physiologic insulin release by dog cell clusters.

FIGS. 22A-22C: Neither MSCs nor cultured pancreatic islet cells contained in allogeneic cell clusters induce antibody formation. In all panels, cells were incubated with sera from control or treated mice and then with Phycoerythrin-labeled (PE) anti-mouse IgG, then analyzed by FACS. Panels A and B depict data from sera that were collected (12 weeks post-treatment) from NOD mice that had been durably rendered euglycemic by cell cluster treatment, or from vehicle-treated or untreated, control NOD mice. FIG. 22A: FACS analysis of C57Bl/6 Gfp+ MSCs from cell clusters. The top row shows histograms of MSCs stained with isotype antibody (negative control, top left), and MSCs incubated with sera from untreated NOD mice (middle and right). The bottom row shows FACS histograms of MSCs incubated with sera from allo-cell cluster-treated (left three panels) or vehicle-treated (right panel) NOD mice. There is no evidence of an antibody response to allogeneic MSCs. FIG. 22B: FACS analysis of C57Bl/6 cultured pancreatic islet cells from cell clusters. The top row shows histograms of pancreatic islet cells stained with isotype antibody (negative control, top left), and pancreatic islet cells incubated with sera from untreated NOD mice (middle and right). The bottom row shows FACS histograms of pancreatic islet cells incubated with sera from allo-cell cluster-treated (left three panels) or vehicle-treated (right panel) NOD mice. These data demonstrate that there is no antibody response to allogeneic, cultured pancreatic islet cells. FIG. 22C: Positive Control. Top histogram: dog ASCs incubated with NOD mouse sera collected 14 days post vehicle treatment, followed by incubation with PE labeled anti-mouse IgG. Bottom histogram: dog ASCs incubated with NOD mouse serum collected 14 days post i.p. treatment with dog ASCs, followed by incubation with PE labeled anti-mouse IgG. These data demonstrate that NOD mice do mount a robust immune response to xenogeneic cells.

FIG. 23: IgG response to the cells used to generate the NIs (MSCs and ICs) that NOD mice were treated with. Shown is a summary of FACS results for P1 C57Bl/6 MSCs and P5 C57Bl/6 cultured ICs incubated with sera and cy3-labeled anti-mouse IgG antibody. Sera were from vehicle-treated and NI-treated NOD mice from the experiment depicted in FIG. 2. Sera were collected at the time of sacrifice (Day 77). As a positive control, sera were also collected from intact C57Bl/6 (allogeneic) islet-treated NOD mice 14 days post i.p. administration of intact, whole islets and assessed by FACS as above. Cy3+: percent of Cy3+ cells (mean±SE) detected upon incubation with sera. A response of <7% was considered to be negative. Antibody mediated rejection of NIs appears unlikely since (i) NOD mice remained euglycemic (see FIG. 2), and (ii) FACS data show no IgG response to these cells in otherwise immune competent NOD mice. *, P<0.05 vs. other treatments.

FIG. 24: Percent of helper, cytotoxic and regulatory T cells from spleens (a-d) and omenta (e) of islet-treated (N=3) vs. NI-treated NOD mice (N=3) 14 days post i.p. administration. For (a) and (b), shown are the percent of CD3+ cells that are also (a) CD4+ or (b) CD8+. For (c) and (d), shown are the percent of CD4+ cells that were also (c) CD25+ or (d) CD25+Foxp3+. While the percentages of helper and cytotoxic T cells were lower in NI treated mice than in islet treated mice, the percentages of regulatory T cells were significantly increased, suggesting that NIs helped restore normoglycemia in NOD mice (see FIG. 2) in part through robust immune-modulation. (e) The percent of Foxp3 positive cells in Omenta of NI-treated NOD mice was markedly increased vs. islet treated animals. Omenta were stained for Foxp3, and percent of positive cells counted as described in Supplementary Information. *, P<0.05 vs. islet-treated group.

FIGS. 25A-25D. Population Doublings (PDL) for human islets cells (hICs) and canine islet cells (cICs). FIG. 25A shows the hours required for a particular number of population doublings for hICs. FIG. 25B the hours required for a particular number of population doublings for hICs obtained from human donors 1-6. FIG. 25C shows the hours required for a particular number of population doublings for cICs. FIG. 25D the hours required for a particular number of population doublings for cICs obtained from canine donors 1-6.

FIGS. 26A-26D. Fold change in relative expression (RQ) as compared to a calibration sample for human islets cell from 8 separate human donors per population doubling (PDL). FIG. 25A depicts the fold change in expression of the insulin (INS) gene. FIG. 25B depicts the fold change in expression of the glucagon (GCG) gene. FIG. 25C depicts the fold change in expression of the somatostatin (SST) gene. FIG. 25D depicts the fold change in expression of the pancreatic polypeptide (PPY) gene.

FIGS. 27A-27D. Fold change in relative expression (RQ) as compared to a calibration sample for canine islets cell from 6 separate canine donors per population doubling (PDL). FIG. 26A depicts the fold change in expression of the insulin (INS) gene. FIG. 26B depicts the fold change in expression of the glucagon (GCG) gene. FIG. 26C depicts the fold change in expression of the somatostatin (SST) gene. FIG. 26D depicts the fold change in expression of the pancreatic polypeptide (PPY) gene.

FIGS. 28A-28C. Glucose sensitive insulin secretion (GSIS) of human islets and culture human islet cells. FIG. 28A shows insulin secretion for human islets and passage 0 (P0) human islet cells when challenged with 5 mM and 25 mM glucose. FIG. 28B shows insulin secretion for cultured islet cells that have undergone 5-7 population doublings (PDLs) when challenged with 5 mM and 25 mM glucose. FIG. 28C shows insulin secretion for cultured islet cells that have undergone 8-11 population doublings (PDLs) when challenged with 5 mM and 25 mM glucose. While the ability to secrete insulin in response to glucose stimulation varies among donors, and culture expansion reduces the level of insulin secretion, hICs continue to secrete insulin in response to glucose, similar to what we previously reported for culture-expanded mouse and dog ICs [2,3].

FIG. 29. Cell clusters formed by overnight co-culture of human islet cells at passages P0-P4 and P3 human MSCs. Scale bar (red)=100 μm. The phenotype and size distribution of formed cell clusters at each tested passage appears comparable. There are only small numbers of single, non-aggregated cells, demonstrating the high efficiency of cell cluster formation

FIG. 30. Dedifferentiation-induced decrease in Islet-specific endocrine gene expression levels of c57Bl/6 mouse ICs normalized to those of whole parent islets and plotted as a function of PDLs in vitro and upon cell cluster retrieval from euglycemic STZ diabetic mice at 21 weeks post administration. Mouse islets were isolated, culture expanded, and assessed for islet-specific endocrine hormone gene expression levels by rtPCR as described in Methods. Mouse ICs at ˜6 PDLs (˜3 weeks) were co-aggregated with murine Mesenchymal Stem Cells to from cell clusters which were used to treat diabetic mice. At 21 weeks post i.p. treatment of STZ-diabetic mice (n=6) with cell clusters (5,000/kg b.wt. given i.p. achieved persistent euglycemia by week 6), gene expression levels of cell clusters retrieved from the mouse omenta were normalized to expression levels of freshly isolated islets (time point PDL 0), indicating effective in vivo redifferentiation (see arrow and symbols representing examined genes). These data demonstrate that although culture expansion of islet cells decreases endocrine gene expression as a function of PDLs, incorporation of ICs into cell clusters and implantation into a diabetic subject results in redifferentiation of the ICs, reestablishment of euglycemia, and restoration of islet hormone gene expression.

FIG. 31. Fold change in relative expression (RQ) of genes of interest as compared to the indicated calibration sample for human islets cells (ICs) from donors 7 and 8 at passage 1 (P1). Black bars depict expression at P1 of cells from donor 7 normalized to expression at P1 of cells from donor 8. Green bars depict expression at P1 of cells from donor 7 normalized to expression in fresh islets from donor 7. Orange bars depict expression at P1 of cells from donor 8 normalized to expression in fresh islets from donor 8. Genes for which expression was measured are insulin (INS), glucacon (GCG), somatostatin (SST), pancreatic peptide (PPT), insulin promoter factor 1 (PDX1), urocortin-3 (UCN3), vascular endothelial growth factor A (VEGFA), C-X-C motif chemokine ligand 12 (CXCL12), transforming growth factor beta 1 (TGFB1), and fibroblast growth factor (FGF2).

FIG. 32. Fold change in relative expression (RQ) of genes of interest as compared to the indicated calibration sample for cell clusters (NIs) as compared to parent human islets cells (ICs) from donors 7 and 8 at passage 1 (P1). Green bars depict expression in cell clusters created with cells from donor 7 normalized to expression at P1 of cells from donor 7. Orange bars depict expression in cell clusters created with cells from donor 8 normalized to expression at P1 of cells from donor 8. Genes for which expression was measured are insulin (INS), glucagon (GCG), somatostatin (SST), pancreatic peptide (PPT), insulin promoter factor 1 (PDX1), urocortin-3 (UCN3), vascular endothelial growth factor A (VEGFA), C-X-C motif chemokine ligand 12 (CXCL12), transforming growth factor beta 1 (TGFB1), and fibroblast growth factor (FGF2).

FIG. 33. Fold change in relative expression (RQ) of genes of interest as compared to the indicated calibration sample for human islets cells (ICs) from donors 7 and 8 at passage 1 (P1). Gray bars depict expression for cell clusters (NIs) generated using cells from donor 7 normalized to cell clusters (NIs) generated using cells from donor 8. Green bars depict expression for cell clusters (NIs) generated using cells from donor 7 normalized to expression in fresh islets from donor 7. Red bars depict expression for cell clusters (NIs) generated using cells from donor 8 normalized to expression in fresh islets from donor 8. Genes for which expression was measured are insulin (INS), glucagon (GCG), somatostatin (SST), pancreatic peptide (PPT), insulin promoter factor 1 (PDX1), urocortin-3 (UCN3), vascular endothelial growth factor A (VEGFA), C-X-C motif chemokine ligand 12 (CXCL12), transforming growth factor beta 1 (TGFB1), and fibroblast growth factor (FGF2).

FIG. 34. Demonstrates the difference in blood glucose over time of between NOD/SCID mice with STZ induced Diabetes mellitus treated with human cell clusters (NI treated, white bars) and controls (vehicle treated controls, black bars).

FIG. 35. Depicts the results of glucose tolerance tests for diabetic NOD/SCID mice treated with human cell clusters (black circles on blue line, human-NI treated), diabetic NOD/SCID mice treated with only the vehicle (black squares on red line on), and non-diabetic NOD/SCID mice (black triangles on green line).

FIG. 36 NI gene expression profiles. Shown are gene expression profiles of freshly prepared human Neo-Islets (hNIs) prior to i.p. administration to diabetic NOD/SCID mice, normalized to expression levels of whole, uncultured human islets. Log 10 (RQ) values were calculated for NIs and graphed as the mean±SEM. Log 10(RQ)±2 (hashed line) was considered statistically significant. Islet associated genes (INS, GCG, SST, PPY, PDX1, and UCN3) are expressed in human NIs prior to administration, but at significantly reduced levels compared to those of freshly isolated human islets

FIGS. 37A-37E Therapeutic efficacy of single and repeat dosing of hNIs administered to STZ-diabetic NOD/SCID mice. FIG. 37A Human Neo-Islets given i.p. only transiently improve blood glucose levels and the i.p. Glucose Tolerance Test (right). FIG. 37B Upon i.p. redosing with the same number of hNIs blood glucose levels, and i.p. Glucose Tolerance Tests (ip GTT; right) are normalized compared to those in non-diabetic NOD/SCID mice, FIGS. 37C and D a response mediated by the exclusive secretion of human Insulin in hNI re-treated NOD/SCID mice (FIG. 37E). As previously reported, murine insulin secretion during the ip GTT in non-diabetic NOD/SCID was physiological.

### DETAILED DESCRIPTION

The disclosed methods, cells, and cell clusters overcome the limited ability to generate sufficient therapeutic doses of isolated and cultured pancreatic islet cells from a single pancreas donor and provide them to a subject in need thereof.

As used herein, Islets may comprise any of the cells found in mammalian pancreatic islets, including but not limited to Alpha cells, Beta cells, Delta cells, Gamma cells, and Epsilon cells. In one embodiment Islets comprise at least insulin expressing Beta cells.

As used herein, cell clusters may comprise Bone Marrow-derived Mesenchymal Stem Cells and/or Adipose-derived Stem Cells, and expanded pancreatic islet cells. The expanded pancreatic islet cells may be dedifferentiated pancreatic islet cells and/or redifferentiated pancreatic islet cells. The redifferentiated pancreatic islet cells may comprise any of the cells found in mammalian pancreatic islets, including but not limited to Alpha cells, Beta cells, Delta cells, Gamma cells, and Epsilon cells. Thus, the cell clusters hereof preferably produce, among other things, insulin, glucagon, somatostatin, pancreatic duodenal homeobox-1, insulin transcription factor mafA, nk6 homeobox-1, etc., which helps to better regulate glucose levels and thus explain the surprisingly good results attained herein. In one embodiment, the cell clusters comprise at least insulin-expressing Beta cells. The cell clusters of the present disclosure may comprise, by way of nonlimiting examples, a ratio of dedifferentiated pancreatic islet cells and/or redifferentiated pancreatic islet cells to adipose stem cells and/or mesenchymal stem cells of 1000:1, 100:1, 50:1, 25:1 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:25, 1:50, 1:100, or 1:1000.

Embodiments include cell clusters, generated in vitro, which are the approximate size of pancreatic islets. Such cell clusters may comprise Bone Marrow-derived Mesenchymal Stem Cells (MSCs) and/or Adipose-derived Stem Cells (ASCs); and expanded pancreatic islet cells. The expanded pancreatic islet cells may be dedifferentiated pancreatic islet cells and/or redifferentiated pancreatic islet cells. Culture expansion may dedifferentiate the pancreatic islet cells via Epithelial-Mesenchymal Transition (EMT), and the resulting cells may be aggregated with MSCs and/or ASCs into the cell clusters, which will spontaneously redifferentiate and resume regulated insulin secretion when administered to subjects. Pancreatic islets, like all organs, possess small numbers of MSCs and/or ASCs that intrinsically, as pericytes, exert robust anti-inflammatory, complex immune-protective, pro-angiogenic, survival and tissue repair-supporting actions. Cell clusters containing dedifferentiated cells may be treated to cause redifferentiation, the redifferentiation resulting in cell clusters comprising redifferentiated pancreatic islet cells that express insulin. In vitro creation of cell clusters, composed of culture expanded pancreatic islet cells and much higher numbers of healthy MSCs and/or ASCs than is physiologic, enable these cell clusters, mediated by the pleiotropic actions of MSCs and/or ASCs, to withstand inflammatory, immune and other insults when administered to subjects with impaired glycemic control, such as seen in Type 1 Diabetes Mellitus, Type 2 Diabetes Mellitus, and other types of insulin-dependent diabetes mellitus, or impaired glucose tolerance.

Isolated pancreatic islet cells (primary pancreatic islet cells) may be from any suitable donor (e.g., rodent, canine, human, or other mammal). In embodiments, the donor is an adult donor.

Islets cells may be obtained from demographically diverse pancreas/islet donors or isolated islets that are not suitable for therapeutic use under the current criteria in use by the medical community—referred to herein as “research grade” islet cells. Islet cells from such donors are generally wasted because they are judged unsuited for an islet transplant. However, cells from such donors, when formed into cell clusters as described herein, are suitable for therapeutic use. In short, the methods and cells clusters described herein provide a major expansion of the size of the donor pool from diverse demographic origin (dog and human) and isolated islets from such donors that do not meet the current quality criteria for a successful islet transplant (e.g., lower cell viability).

In aspects, the pancreatic islet cells used here may be classified as “research grade,” i.e., not intended for therapeutic use. In additional embodiments, the pancreatic islet cells may be obtained from a donor having a North America Islet Donor Score (NAIDS) of less than 80, less than 75, less than 70, less than 65, less than 60, less than 55, less than 50, less than 45, less than 40, less than 35, less than 30, less than 25, less than 20, less than 15, or less than 10 as defined by Golçbiewska, et al., and Yeh, et al. [4,5]. Specifically included herein are methods for the treatment of subjects with impaired glycemic control, such as Type 1 Diabetes Mellitus, Type 2 Diabetes Mellitus, and other types of insulin-dependent diabetes mellitus, or impaired glucose tolerance by the i.p. administration of the cell clusters described herein where the clusters contain pancreatic islet cells that were expanded from cells classified as research grade or having a NAIDS score as indicated above.

Differentiated pancreatic islet cells express, e.g., insulin, but do not proliferate, or proliferate only minimally in vitro. Isolated pancreatic islet cells may be induced to dedifferentiate in vitro. As used herein, “dedifferentiated” pancreatic islet cells or islet cell nuclei are cells or nuclei that no longer express or produce physiological levels of insulin when challenged with glucose. In certain embodiments, the expression of insulin by dedifferentiated pancreatic islet cells when challenged with glucose may be reduced by 10%, 20%, 30%, 40%, 50%, 60%, 70%, 80%, 90%, or more as compared to primary isolated pancreatic islet cells. The process of dedifferentiation is also referred to herein as an Epithelial-Mesenchymal transition or an “E to M” transition. Dedifferentiated pancreatic islet cells may proliferate in culture at a rate superior to differentiated pancreatic islet cells. Dedifferentiation of the pancreatic islet cells may immediately reduce or silence insulin expression, insulin synthesis, insulin storage, and/or glucose-induced insulin secretion in these cells.

Dedifferentiated pancreatic islet cells may be allowed to proliferate in vitro to form a large pool of cells that may be co-cultured and/or formed into cell clusters with other cell types.

Proliferation associated dedifferentiation may be achieved by culturing pancreatic islet cells in conditions which are adherent for the pancreatic islet cells. In various embodiments, the pancreatic islet cells may be cultured on a surface that has been coated with or not coated with laminin 511 or laminin 411. Dedifferentiation may optionally be performed in a dedifferentiation medium. Dedifferentiation medium may include a glucagon-like peptide 1 (GLP-1) receptor agonist. In specific embodiments, the GLP-1 receptor agonist may be GLP-1, exenatide, liraglutide, lixisenatide, albiglutide, taspoglutide, and/or Exendin-4. The GLP-1 receptor agonist may be present in the dedifferentiation culture medium at a concentration from 0.1 to 100 nM, from 1 to 50 nM, or at 1, 2, 3, 4, 5, 6, 7, 8, 9, 10, 11, 12, 13, 14, 15, 16, 17, 18, 19, 20, 25, 26, 27, 28, 29, or 30 nM.

In embodiments, the dedifferentiated islets cells may be expanded in culture for at least 1 population doubling prior to inclusion in a cell cluster. Numbers of population doublings that can be undergone include, but are not limited to 1, 2, 3, 4, 5, 6, 7, 8, 9, 10, 11, 12, 13, 14, 15, 16, 17, 18, 19, 20, 21, 22, 23, 24, 25, 30, 35, 40, 45, and 50 population doublings before inclusion into a cell cluster.

In particular embodiments, dedifferentiated islets cells may be redifferentiated prior to inclusion in cell clusters described herein.

Culturing of isolated pancreatic islets and/or pancreatic islet cells on laminins (e.g., Laminin 411 and 511), and addition of suitable media, may improve cell adhesion in culture, support cell survival, and moderately boost proliferation. For dedifferentiation, pancreatic islet cells may be plated on a suitable substrate that allows for attachment. In specific embodiments, the substrate may include Laminin 411 and/or Laminin 511. In a more specific embodiment, islet cells may be plated on tissue culture flasks or wells coated with Laminin-411 and/or Laminin-511 and placed in RPMI, DMEM, alpha MEM, CMRL, PIM, or other suitable culture media and supplemented with 10% to 20% fetal bovine serum or other species-specific serum or platelet lysate, and glutamine/penicillin/streptomycin. The culture medium may also be supplemented with at least 10 nM Exendin-4.

Examples of sera in which the cell clusters may be cultured include, but are not limited to, sera available from worldwideweb.sigmaaldrich.com. Specific non-limiting examples include: Fetal Bovine Serum, Bovine Calf Serum, Adult Bovine Serum, Chicken Serum, Goat Serum, Porcine Serum, Rabbit Serum, Sheep Serum, Horse Serum, Canine Serum, Baboon Serum, Coyote Serum, Goose Serum, Mouse Serum, Rat Serum, Rhesus Monkey Serum, Serum Replacement, and Human Serum.

Included are methods of making the cell clusters, the methods comprising: expanding pancreatic islet cells as described herein; and forming cell clusters comprising: the expanded pancreatic islet cells; and mesenchymal stem cells and/or adipose stem cells.

The MSC/ASC component of the cell clusters provides immune isolation, protection, and increased survival of the islet-derived component (the dedifferentiated pancreatic islet cells or redifferentiated pancreatic islet cells), thereby preventing rejection and enhancing engraftment of the cell clusters. Amplification via significantly increased numbers of cells of the potent immune-modulating activities of normal MSCs and/or ASCs in cell clusters provides auto- and allo-immune isolation of pancreatic islet cells, thereby eliminating the need for anti-rejection drugs or encapsulation devices. Consequently, in certain embodiments of treating a subject with the cells described herein, anti-rejection drugs are not administered to the patient. In further embodiments, the cells described herein are not encapsulated and/or associated with an encapsulation device. Moreover, the MSC/ASC component of the cell cluster may induce, via the release of hepatocyte growth and other factors, reversal of the Epithelial to Mesenchymal transition, thus facilitating redifferentiation of dedifferentiated pancreatic islet cells into insulin and other islet hormone producing cells in vivo.

In further embodiments, the cell clusters are administered intra-peritoneally (i.p.). The ability of the mammalian omentum to take up foreign bodies and various cell types facilitates the durable and spontaneous engraftment of the cell clusters, which then deliver insulin to the subject physiologically, i.e., into the portal vein of the liver, additionally optimized by superior peritoneal glucose sensing and oxygen pressures to that in the subcutaneous and portal vein spaces (see, D. R. Burnett, L. M. Huyett, H. C. Zisser, F. J. Doyle, and B. D. Mensh, “Glucose sensing in the peritoneal space offers faster kinetics than sensing in the subcutaneous space,” Diabetes 63:2498-505 (2014), incorporated herein by this reference [6]). The physiological route of insulin delivery might reduce insulin resistance, insulin-enhanced lipogenesis and potentially harmful exposure of peripheral tissues to high concentrations of insulin. For these reasons the omentum is uniquely suited for implantation of the cell clusters, in addition, should the need arise the cell clusters can be removed from the subject via an omentectomy (surgical removal of part or all of the omentum).

In further embodiments, should there be evidence for premature rejection of cell clusters, a short initial course with rapamycin or other suitable anti-rejection agent may administered to the subject to improve cell cluster survival and function. If a recipient of this therapy lacks or has a damaged omentum, an intra portal vein transplant, other location, or a suitable encapsulation device may be utilized.

In each of the above examples of methods, cell clusters may be coated with hydrogel. Such coating may be performed after any step in which a cell cluster is formed or prior to infusing or providing cell clusters to a subject.

In each of the above examples of methods, cell clusters may be contained within an encapsulation device. Such encapsulation may be performed after any step in which a cell cluster is formed or prior to infusing or providing cell clusters to a subject

In various embodiments, the cell clusters may be immune privileged. As used herein, “immune privileged” refers to cell clusters described herein eliciting no or a less robust immune response than cells or cell clusters that are not immune privileged. In various embodiments, the immune response to “immune privileged” cells or cell clusters may be 0, 5, 10, 20, 30, 40, 50, 60, 70, 80, 90, 95% or 100% or less than the immune response to non-immune privileged cells or cell clusters.

MSCs and ASCs are undifferentiated, multipotent, adult stem cells, also known as stromal cells that proliferate well, and do not produce insulin. MSCs and ASCs may be from any suitable donor (e.g., rodent, canine, human, or other mammal).

Dedifferentiated pancreatic islet cells proliferate well, but do not, or only minimally express or secrete insulin. In some embodiments, dedifferentiated pancreatic islet cells are allowed to proliferate to generate sufficient numbers for subsequent manipulation. In certain embodiments, once sufficient dedifferentiated pancreatic islet cells have been generated the cells are treated with an islet cell or beta cell-specific redifferentiation medium. Redifferentiation of the pancreatic islet cells restores insulin production, resulting in the re-expression of physiological insulin expression, synthesis, storage, and glucose-sensitive insulin release.

Described is the redifferentiation of dedifferentiated pancreatic islet cells to generate a redifferentiated islet cell. Redifferentiation, as used herein, refers to the treatment of dedifferentiated pancreatic islet cells to generate a redifferentiated islet cell having restored expression of physiological insulin expression, synthesis, storage, and glucose-sensitive insulin release. In certain embodiments, redifferentiation may be a two-step process.

In a first step, a dedifferentiated islet cell may be exposed to a culture medium containing a low level of glucose. The low level of glucose may be selected from 1, 2, 3, 4, 5, 5.1, 5.2, 5.3, 5.4, 5.5, 5.6, 5.7, 5.8, 5.9, and 6 mM D-glucose. The medium may contain other components such as Insulin/Transferrin/Selenium (ITS), penicillin/streptomycin (Pen/Strep), fetal bovine serum (FBS), dog serum, or human platelet lysate. The first step may include culturing the dedifferentiated islet cell in the culture medium containing a low level of glucose for 1, 2, 3, 4, 5, 6, 7, 8, 9, 10, 11, 12, 13, 14, 1 to 14, 2 to 13, 3 to 12, 4 to 10, or 5 to 9 days.

In a second step, the dedifferentiated islet cell may be exposed to a culture medium containing a high level of glucose. The high level of glucose may be selected from 15, 16, 17, 18, 19, 20, 22, 23, 24, 25, 26, 27, 28, 29, 30, 31, 32, 33, 34, and 35 mM D-glucose. The medium may contain other components such as insulin/transferrin/selenium (ITS), penicillin/streptomycin (Pen/Strep), fetal bovine serum (FBS), dog serum, or human platelet lysate, N2 supplement, B27 supplement, nicotinamide, Activin A, Alk-5 inhibitor II, triiodothyronine, and a glucagon-like peptide 1 (GLP-1) receptor agonist. Nicotinamide may be present in the culture medium at a concentration from 0.1 to 100 mM, from 1 to 50 mM, or at 1, 2, 3, 4, 5, 6, 7, 8, 9, 10, 11, 12, 13, 14, 15, 16, 17, 18, 19, 20, 25, 26, 27, 28, 29, or 30 mM. Activin A may be present in the culture medium at a concentration from 0.1 to 100 mM, from 1 to 50 mM, or at 1, 2, 3, 4, 5, 6, 7, 8, 9, 10, 11, 12, 13, 14, 15, 16, 17, 18, 19, 20, 25, 26, 27, 28, 29, or 30 mM. The GLP-1 receptor agonist may be present in the culture medium at a concentration from 0.1 to 100 nM, from 1 to 50 nM, or at 1, 2, 3, 4, 5, 6, 7, 8, 9, 10, 11, 12, 13, 14, 15, 16, 17, 18, 19, 20, 25, 26, 27, 28, 29, or 30 nM. The Alk-5 inhibitor II may be present in the culture medium at a concentration from 1, 2, 3, 4, 5, 6, 7, 8, 9, 10, 11, 12, 13, 14, 15, 16, 17, 18, 19, 20, 25, 26, 27, 28, 29, or 30 μM. The triiodothyronine may be present in the culture medium at a concentration from 0.1 to 100 μM. The GLP-1 receptor agonist may be Exendin-4. The second step may include culturing the dedifferentiated islet cell in the culture medium containing a high level of glucose for 10, 12, 13, 14, 15, 16, 17, 18, 19, 20, 21, 22, 23, 24, 25, 26, 27, 28, 10 to 28, 11 to 27, 12 to 26, 13 to 25, or 14 to 29 days.

In some embodiments, a method is provided for generating insulin-producing cells through a substantial expansion in the amount of starting material (dedifferentiated pancreatic islet cells) for subsequent culturing with proliferating MSCs or ASCs.

Methods are disclosed for the formation of the cell clusters as described herein. Such cell clusters may be approximately the size of islets found in the pancreas. Cell clusters may be formed, e.g., by any method known in the art. In a non-limiting example, cell clusters are formed by the culturing of cells on hydrophobic, ultra-low adhesion surfaces.

Examples of hydrophobic and/or ultra-low adhesion surfaces include, but are not limited to untreated polystyrene, low attachment hydrogel layers, and uncharged surfaces. Also described are methods of treating a subject in need of insulin and/or suffering from Type 1 (“T1DM”) or Type 2 Diabetes Mellitus (“T2DM”), or suffering from impaired glucose tolerance or Prediabetes Mellitus, using the described cell clusters is disclosed. In some embodiments, cell clusters are administered intraperitoneally (i.p.) and/or to the omentum of the subject. In certain embodiments, cell clusters are administered s.c., or otherwise parenterally to the subject. In certain embodiments, administration of the cell clusters to the subject increases and/or restores insulin production, secretion, and glucose-responsiveness. In certain embodiments, the cell clusters may be coated with hydrogel or other FDA approved material prior to administration to further enhance survival of the cell clusters in vivo, such as gelfoam, or a thrombin clot. In embodiments where the cell clusters contain dedifferentiated pancreatic islet cells, these cells may undergo redifferentiation in the subject after treatment of the subject with the cell clusters.

Methods of treating subjects with cell clusters comprise providing a dose of cell clusters comprising a therapeutically sufficient number of the cell clusters to a subject suffering from T1DM, T2DM, or impaired glucose tolerance to increase and/or restore insulin production, secretion, and glucose-responsiveness. This dose would be understood by those of ordinary skill in the art to vary depending on the route of administration, the weight of the subject, the degree of pathology in the subject to be treated, and the subject's response to therapy. In certain embodiments, subsequent doses of cell clusters could be administered to the subject depending on their initial response to therapy. In embodiments, a therapeutically sufficient number of cell clusters comprises sufficient expanded pancreatic islet cells to increase and/or restore insulin production, secretion, and glucose-responsiveness. In particular embodiments, a therapeutically sufficient number of the cell clusters comprises at least 1.00E+01, 1.00E+02, 1.00E+03, 1.00E+04, 1.00E+05, 1.00E+08, 2.00E+08, 3.00E+08, 4.00E+08, 5.00E+08, 7.00E+08, 8.00E+08, 9.00E+08, 1.00E+09, 2.00E+09, 3.00E+09, 4.00E+09, 5.00E+09, 7.00E+09, 8.00E+09, 9.00E+10, 1.00E+10, 2.00E+10, 3.00E+10, 4.00E+10, 5.00E+10, 7.00E+10, 8.00E+10, 9.00E+10, 1.00E+11, 1.00E+12, 1.00E+13, 1.00E+14, 1.00E+15, 1.00E+16, 1.00E+17, 1.00E+18, 1.00E+19, or 1.00E+20 expanded pancreatic islet cells.

The high efficiency (i.e. the very small loss of viable cells) of the methods described herein also provides a significant increase in the number of doses that can be obtained from a single pancreas over currently conventional treatment. For example, based on the average number of islets that can be obtained from a single human pancreas, expanding the pancreatic islet cells as described herein may provide, from a single donor, sufficient pancreatic islet cells for 10, 25, 50, 75, 100, 250, 500, 750, 1000, 1250, 1500, 1750, 2000, 3000, 4000, 5000, 6000, 7000, 8000, 9000, 10000, 20000, 30000, 40000, 50000, 60000, 70000, 80000, 90000, 100000 or more doses of cell clusters sufficient to increase and/or restore insulin production, secretion, and glucose-responsiveness. In contrast, current human islet transplants require approximately 3-5 pancreata for a single human dose. Further, repeat doses are often needed to reestablish insulin independence.

“Treating” or “treatment” does not require a complete cure. It means that the symptoms of the underlying disease are at least reduced, and/or that one or more of the underlying cellular, physiological, or biochemical causes or mechanisms causing the symptoms are reduced and/or eliminated. Insulin requirements may be reduced. End organ damage may be reduced. The need for anti-rejection drugs may be reduced or eliminated. It is understood that reduced, as used in this context, means relative to the state of the disease, including the molecular state of the disease, not just the physiological state of the disease.

The treatment (especially in the early stages) may be aided by the administration of insulin and/or oral hypoglycemic agents (or drugs). Such drugs include the biguanides (e.g., metformin), sulfonylureas (e.g., glimepiride, glyburide, or glipizide), meglitinides (e.g., repaglinide), diphenylalanine derivatives (e.g., nateglinide), thiazolidinediones (e.g., pioglitazone), DPP-4 inhibitors (e.g., sitagliptin, saxagliptin, linagliptin), alpha-glucosidase inhibitors (e.g., acarbose or miglitol), bile acid sequestrants (e.g., colesevelam), etc. Dosages and administration of such drugs, adjuvants and/or intermediate treatment(s) would be readily determined by a person of ordinary skill in the art and dependent on the subject being treated, and need not be repeated here.

Also described are methods of preparation and packaging the cell clusters known in the art to allow for preparation of the cell clusters remotely from the subject to be treated while ensuring survival of the cell clusters before administration, further enhancing survival of the cell clusters in vivo after administration. For instance, various implants are well known to those of ordinary skill in the art. Encapsulation and microencapsulation devices and methods are also well known.

Packaging may be accomplished, for example, by means known in the art, such as packaging fresh or frozen cell clusters into, e.g., syringes, sterile bags, infusion bags, bottles, etc., for delivery to a subject or health care practitioner. Plasmalyte A pH 7.4 maybe extremely useful in packaging the cell clusters.

The use of animal models, including rodent and canine models, is well understood by those of ordinary skill in the art to provide a useful tool in developing treatments for human diabetes [7]. Indeed, as King notes, it is ideal to provide more than one animal model to better represent the diversity of human diabetes, as is disclosed herein. The description provided would enable those of ordinary skill in the art to make and use cell clusters to treat T1DM, T2DM, and impaired glucose tolerance in humans without any undue experimentation.

In some embodiments, the subject may be a mammal, such as, for example, a rodent, dog, cat, horse, or human. In further embodiments, cells in the cell cluster may be allogenic, xenogenic, or a combination of allogenic and xenogenic cells in relation to the subject or other cells in the cell cluster.

As used herein, “comprising,” “including,” “containing,” “characterized by,” and grammatical equivalents thereof are inclusive or open-ended terms that do not exclude additional, unrecited elements or method steps, but also include the more restrictive terms “consisting of” and “consisting essentially of.”

### EXAMPLES

The following examples are provided for illustration purposes only and are not to be construed as limiting the disclosure to the embodiments specifically disclosed therein.

Since Pancreatic islets, like all tissues, possess small numbers of Mesenchymal Stem Cells, as pericytes, that exert immune-modulating, anti-inflammatory and other protective trophic effects locally, [8-14] we hypothesized and tested whether cell clusters (Neo-Islets (NIs)) comprising endocrine pancreatic islet cells with much higher numbers of MSCs/ASCs could be formed, and whether such cell clusters would provide effective: (i) Auto- and/or allo-immune-Isolation without encapsulation devices, (ii) Survival benefits of allogeneic cell clusters in vivo, thereby reducing or eliminating the need for anti-rejection drugs, (iii) Redifferentiation in vivo of pancreatic islet cells, and thereby (iv) Adequate and physiologic insulin secretion and durable maintenance of euglycemia in rodents with T1DM.

The unique and well documented pleiotropic and largely comparable actions of bone marrow-derived Mesenchymal Stromal Cells (MSCs) or adipose tissue-derived Adipose Stem Cells (ASCs), if combined with equal numbers of pancreatic islet cells in islet-sized cell clusters or “cell clusters” (NI), are harnessed to shield administered β-cells from allo- and auto-immune attacks and inflammatory damage, and to enhance β-cell survival and induce angiogenesis. Physiologically, only about 2% of the total cell numbers in islets are MSCs, located as pericyte-like cells in microvascular niches. Their cytoprotective functions within the islets likely parallel those in the bone marrow and other organs, i.e., vasculo-protection and stabilization, anti-inflammatory, trophic and immune-modulating activities. Such NIs of approximate islet size were generated in vitro from culture expanded, via Epithelial-Mesenchymal Transition (EMT) and associated dedifferentiation, pancreatic islet cells and bone marrow-derived MSCs of C57Bl/6 mice. 5×10³ NIs, each composed of ˜500 pancreatic islet cells and ˜500 MSCs, were intraperitoneally (i.p.) administered to spontaneously diabetic, immune-competent NOD mice that develop an auto-immune form of T1DM that largely resembles human T1DM. This allogeneic treatment protocol was chosen as it models the most common clinical situation in recipients of pancreas or islet transplants. By not using anti-rejection drugs or encapsulation devices, we rigorously tested that high numbers of MSCs in NIs do enable pancreatic islet cells to survive, redifferentiate into normally functioning endocrine cells, and thereby durably establish glycemic control in NOD mice with autoimmune T1DM. While NI treated diabetic NOD mice thrived normally, vehicle treated, diabetic NOD mice remained hyperglycemic and began to die. These initial data implied that NIs survive, engraft in the omentum, and redifferentiate into functional endocrine cells in vivo, and that both allo- and auto-immune protection is achieved. Importantly, following i.p. administration the NIs were taken up by the omentum where they engrafted long term and redifferentiated into physiologically insulin- and other islet-hormone-producing cells. NOD mice did not mount a humoral allo-immune response to the MSCs and pancreatic islet cells that are used to form NIs. NI-treated diabetic animals showed a significant increase in regulatory T cell (Treg) numbers in their omenta and spleens compared to animals that were treated with islets. When NIs were injected into nondiabetic animals, they also engrafted and survived in the omentum without causing hypoglycemia, further demonstrating regulated insulin secretion. Insulin secretion from the omentum occurs into the portal system of the liver, as does that from the pancreas, which is physiologic and results in inactivation of ˜50% of the delivered insulin. This limits the post-hepatic exposure of muscle, adipose tissue, the vasculature and other organs to supraphysiological, potentially hypoglycemia-inducing and otherwise harmful insulin levels that are generated when insulin is subcutaneously given. When streptozotocin (STZ) diabetic mice were treated with similarly sized cell clusters composed of either only MSCs or Islet cells, blood glucose levels, compared to NI treated, euglycemic animals, were only minimally lowered compared to vehicle treated controls. This clearly demonstrated that the therapeutic efficacy of NIs depends critically on the collaboration of MCSs and pancreatic islet cells. Finally, when STZ-diabetic NOD/SCID mice were treated i.p. by identical protocol with canine NIs (cNI), euglycemia was readily and durably induced and intraperitoneal Glucose Tolerance Tests (IP GTT) were normalized. Importantly, the insulin that was released during the IP GTT was canine specific, and when cNIs were surgically removed, hyperglycemia redeveloped. Taken together, the present data demonstrate that the complex pleiotropic actions of MSCs or ASCs (M/ASCs), as hypothesized, can be readily harnessed to protect cultured pancreatic islet cells, and when combined with them in NIs and administered i.p., facilitate long term glycemic control in mice with autoimmune T1DM. We conclude, therefore, that these observations have significant translational relevance for the treatment of T1DM.

Reagents: Reagents used and their sources are listed in the following table.

| Reagent | Source |
|---|---|
| 20 mM citrate buffer pH 4.5 | Sigma, St. Louis, MO |
| 4′,6-diamidino-2-phenylindole | Life Technologies, Carlsbad, CA |
| dihydrochloride | |
| Accumax | Innovative Cell Technologies, |
| | Inc., San Diego, CA |
| ACK buffer | Life Technologies, Carlsbad, CA |
| Anti-Ki67 rabbit IgG monoclonal | Abcam, Cambridge, MA |
| antibody (ab16667) | |
| Bovine Serum Albumin (BSA) | Sigma, St. Louis, MO |
| Canine IFN-gamma (781-CG-050) | R&D Systems, Minneapolis, MN |
| Canine Insulin ELISA kit | Mercodia, Uppsala, Sweden |
| Canine serum | Golden West Biologicals, |
| | Temecula, CA |
| Click-iT EdU Alexa Fluor | Invitrogen, Carlsbad, CA |
| 594 Imaging Kit | |
| Cell Tracker Green | Life Technologies, Carlsbad, CA |
| Collagenase 1 | Worthington, Lakewood, NJ |
| Collagenase P | Roche, Indianapolis, IN |
| Cy3 conjugated goat | Jackson ImmunoResearch, |
| anti-rabbit IgG (111116003) PA | West Grove, |
| Cy3-conjugated | Jackson ImmunoResearch, |
| goat-anti-mouse IgG ab PA | West Grove, |
| DAB substrate Staining (SK-4100) | Vector Laboratories, |
| | Burlingame, CA |
| DiI | Life Technologies, Carlsbad, CA |
| DIR | Life Technologies, Carlsbad, CA |
| dithizone | Sigma, St. Louis, MO |
| DMEM-F12 | Sigma, St. Louis, MO |
| DMSO | Sigma, St. Louis, MO |
| donkey anti-guinea pig cy5 | Jackson ImmunoResearch, |
| conjugated antibody PA | West Grove, |
| Eosin Y Solution | Sigma, St. Louis, MO |
| Fetal Bovine Serum (FBS) | Hyclone, Logan, UT |
| Fluorescein diacetate | Sigma, St. Louis, MO |
| Formalin | Sigma, St. Louis, MO |
| Gelfoam | Pfizer, Kalamazoo, MI |
| Gentamycin Penicillin | Sigma, St. Louis, MO |
| Streptomycin (GPS) | |
| guinea pig anti-insulin antibody | Dako, Carpinteria, CA |
| Hanks Buffered Saline Solution | Gibco, Carlsbad, CA |
| Hematoxylin counterstain (H-3404) | Vector Laboratories, |
| | Burlingame, CA |
| HEPES | Gibco, Carlsbad, CA |
| Histopaque 1077 | Sigma, St. Louis, MO |
| Histopaque-1.119 | Sigma, St. Louis, MO |
| Isoflurane | Baxter, Deerfield, IL |
| Laminin-511 | BioLamina, Uppsala, Sweden |
| L-Glutamine-Penicillin- | Sigma, St. Louis, MO |
| Streptomycin (GPS) | |
| Linbits | LinShin Canada, Toronto, |
| | Ontario, Canada |
| Mouse T Lymphocyte Subset | BD Pharmingen, San Jose, CA |
| Antibody Cocktail (#558391) | |
| NaHCO₃ | Sigma, St. Louis, MO |
| OneTouch Ultra2 Glucometer | Johnson and Johnson, |
| | New Brunswick, NJ |
| PBS | Roche, Indianapolis, IN |
| Propidium Iodide | Life Technologies, Carlsbad, CA |
| Qiagen RNeasy Mini Kit | Qiagen, Germantown, MD |
| rabbit anti Foxp3 antibody | Abcam, Cambridge, MA |
| (ab54501) | |
| RPMI 1640 | Life Technologies, Carlsbad, CA |
| Stempro Osteo-, Chondro-, | Gibco, Carlsbad, CA |
| Adiogenic differentiation kits | |
| Streptozotocin | Sigma, St. Louis, MO |
| SuperScript II Reverse Transcriptase | Invitrogen, Carlsbad, CA |
| TaqMan PCR primers | Applied Biosystems, |
| | Foster City, CA |
| TaqMan Universal Master | Applied Biosystems, |
| Mix II with UNG | Foster City, CA |
| Tregs Detection Kit (#130-094-165) | Miltenyi Biotec, Bergisch |
| | Gladbach, Germany |
| Triton X 100 | Fisher Scientific, Waltham, MA |
| Trypsin EDTA | Sigma, St. Louis, MO |

### Example 1: Islet Isolation

From Rodents: Mice were euthanized with Isoflurane (3-5%) in a sealed chamber, and immediately placed on a surgical board for a sterile midline incision. The pancreas was exposed, the pancreatic duct located. The common bile duct was clamped, and the pancreas was inflated with 5 ml/mouse or 15 ml/rat 1 mg/ml Collagenase P in Dissociation Buffer (Hanks Buffered Saline Solution (HBSS), Ca⁺⁺, Mg⁺⁺+25 mM HEPES+NaHCO₃) via the common bile duct. The inflated pancreas was removed to a sterile conical tube containing digestion solution (1 mg/ml Collagenase P in Dissociation Buffer.). The tube was placed in a 37° C. shaking water bath (120 rpm) and the contents digested for 15 minutes. The digestion was stopped with an equal volume of cold Dissociation Buffer. The digested tissue was filtered through a 400 μm screen into a fresh tube, and centrifuged at 1200 rpm for 2 minutes at 4° C. with the brake off. The pellet was washed with 20 ml Dissociation Buffer and centrifuged again (1200 rpm for 2 minutes at 4° C. with the brake off). To purify the islets further, the pellet was resuspended in 10 ml Histopaque 1077 solution and overlayed with 10 ml serum free DMEM-F12 to set up a gradient. The gradient was centrifuged at 2000 rpm for 20 minutes at 4° C. with the brake off, and the islets were collected at the interface between the medium and Histopaque into a 50 ml conical tube containing 20 ml Dissociation Buffer. The islets were then centrifuged at 1200 rpm for 2 minutes, washed with 20 ml Dissociation Buffer, spun down again, resuspended in islet culture medium, and placed in a sterile Petri dish. Islets were allowed to recover in a 37° C., 5% CO₂ humidified incubator at pH 7.4 overnight.

From Dogs: Fresh pancreata were obtained from euthanized dogs through an NIH sharing agreement and inflated via the common bile duct, using 1 mg/ml Collagenase P solution. Canine islets were isolated from inflated pancreases following modified versions of techniques described by Vrabelova, et al. and Woolcott, et al.[15,16] In brief, the distended dog pancreas was cut in 15 to 20 pieces and placed in a 50 ml tube containing 20 ml of 1 mg/ml Collagenase P solution. The tube was placed into a 37° C. water bath with the shaker set at 120 rpm. Islet content in the solution was monitored by microscopic examination of dithizone stained samples obtained from small samples taken at 5-minute intervals. Digestion was continued until approximately 50% of islets were free of acinar tissue, and stopped with 20 ml of HBSS supplemented with 10 mM HEPES+1% BSA. The tissue was then gently sieved through a 400-μm screen and centrifuged for 10 seconds at 100×g at 4° C. The pellets were washed once and centrifuged for 10 seconds at 200×g (4° C.). Three layer density gradients were created by resuspending the pellets in 10 ml Histopaque-1.119, slowly layering on top 10 ml of Histopaque-1.077 followed by another layer of 10 ml of serum-free medium. The gradient was spun at 750×g for 20 minutes at 4° C. without brake. Islets were collected from the top interface and transferred to a 50 ml tube containing HBSS supplemented with 10 mM HEPES+1% BSA. The purified islet suspensions were washed with serum-free medium and centrifuged for 10 seconds at 200×g (4° C.) twice and passed through a 40-μm cell strainer. Five 50 μl aliquots from each preparation were collected and used to assess the islet yield Finally, hand-picked (to remove acinar cell content) islets were cultured in 20% FBS supplemented RPMI 1640 medium at 37° C., in a 5% CO₂ incubator.

From Humans: Human islets were purchased from Prodo Laboratories (Irvine, CA) or obtained from other legitimate sources of human donor tissue.

### Example 2: Culture and De-Differentiation of Pancreatic Islet Cells

Rodent Islet Cells: Recovered mouse islets were hand-picked and further purified by capturing the islets in the top of a 40 μm filter strainer. Islets were cultured as follows: pancreatic islet cells were cultured by placing whole islets on Laminin-511 coated wells, and allowing the pancreatic islet cells to outgrow from the islets until 90% confluent in RPMI 1640+20% FBS+GPS, which results in their dedifferentiation via reversible EMT. Culturing in this manner further purifies pancreatic islet cells and removes remaining exocrine cells. Passaging: Mouse pancreatic islet cells were allowed to grow to approximately 90% confluence. They were then trypsinized (1× Trypsin-EDTA for 5-10 minutes), pelleted by centrifugation at 600×g for 5 minutes, washed with DMEMF12+20% FBS+GPS, and seeded into T75 flasks. Passaged pancreatic islet cells were cultured in DMEM-F12+20% FBS+GPS. Culturing in this manner further purifies pancreatic islet cells and removes acinar and ductal cells.

Canine Islet Cells: Initial Culture: Recovered dog islets were handpicked and further purified by capturing the islets in the top of a 40-μm filter strainer. Cells were cultured as whole islets as described above for mice. Passaging: see as above for rodent pancreatic islet cells.

Human Islet Cells: Cells were cultured as whole islets and passaged as described above for rodents.

### Example 3: Isolation and Culture of ASCs and MSCs

ASCs (mouse and canine): Under sterile conditions, approximately 3-15 g abdominal fat samples were harvested from euthanized, non-diabetic mice or non-diabetic dogs (NIH tissue sharing agreement) and placed on ice in separate, sterile 50 ml conical tubes containing approximately 30 ml of 1×PBS. The fat samples were minced, placed in tubes of PBS containing 3 mg/ml Collagenase 1, and digested approximately 1 hour in a 37° C. shaking water bath. The tubes were centrifuged (600×g, 10 minutes) to pellet the cellular content. The supernatant was carefully removed, and the pellet washed two times with sterile PBS, and then resuspended in 10 ml DMEM F12+GPS+10% FBS for culture. Cells were cultured in a 37° C. humidified 5% CO₂ incubator at pH 7.4. Culture medium was changed twice weekly. When primary cultures reached 70-80% confluence, attached cells were passaged by exposure to 1× trypsin/EDTA for 3-5 minutes, and further passaged or cryopreserved in 10% DMSO.

Non-diabetic Human ASCs were purchased at P1 from Lonza (Walkersville, MD), and cultured as described above.

MSCs (from rodents): Obtained cell suspensions from flushed femurs of euthanized mice were plated in T25 flasks containing DMEM-F12+10% FBS+GPS. Cells were cultured in a 37° C. humidified 5% CO₂ incubator. Culture medium was changed twice weekly. When primary cultures reached 70-80% confluence, cells were detached with 1× trypsin/EDTA for 3-5 minutes, and passaged or cryopreserved in 10% DMSO.

Prior to cell cluster formation, cultured MSCs or ASCs are characterized (i) by FACS for their expression of CD44 and CD90, and negative expression of CD45, CD34 and DLA-DR antigens, and (ii) by their abilities to undergo trilineage differentiation (adipogenic, osteogenic, chondrogenic) as previously described.[17] Prior to cell cluster formation, cultured, dedifferentiated canine pancreatic islet cells are examined by (a) FACS and confirmed to be negative for expression of DLA-DR, CD90 and CD133; and (b) rtPCR for residual islet cell gene expression of insulin, glucagon, somatostatin, pancreatic polypeptide, pdx-1, and nkx6.1. Cell viability was assessed using Fluorescein diacetate (FDA) and Propidium Iodide (PI) as follows: 1× staining solution (1 μL of 5 mg/ml FDA and 5 μL of 1 mg/ml PI dissolved in 100 μL PBS) was mixed with cells in 100 μL PBS, incubated at room temperature for seconds and cells were imaged using a fluorescence microscope. Four fields were counted for red, green and total cell numbers.

### Example 4: Induction of Indoleamine 2, 3 Dioxygenase (IDO-1)

Canine ASCs were tested at P2 for induction of IDO-1 in response to canine interferon gamma (IFNγ) as follows. Eight 35 mm culture dishes were seeded with 0.5×10⁶ canine-derived ASCs each in DMEM F12+10% canine serum. 10 ng/ml canine INFγ was added to four dishes. After overnight culture in a 37° C. humidified 5% CO₂ incubator, cells from all dishes were harvested and assayed for IDO-1 gene expression by rtPCR. Results from IFNγ treated cultures were normalized to those of unexposed cells of the same passage number and expressed as Log 10RQ.

### Example 5: Cell Cluster Formation and In Vitro Characterization

Rationale: (A) To test whether cell clusters comprising (i) dedifferentiated, culture expanded pancreatic islet cells combined with (ii) much higher numbers of MSCs/ASCs than occurs naturally in islets could be formed. (B) To determine whether and to what extent such cell clusters express or can be induced to express islet cell associated genes.

Methods

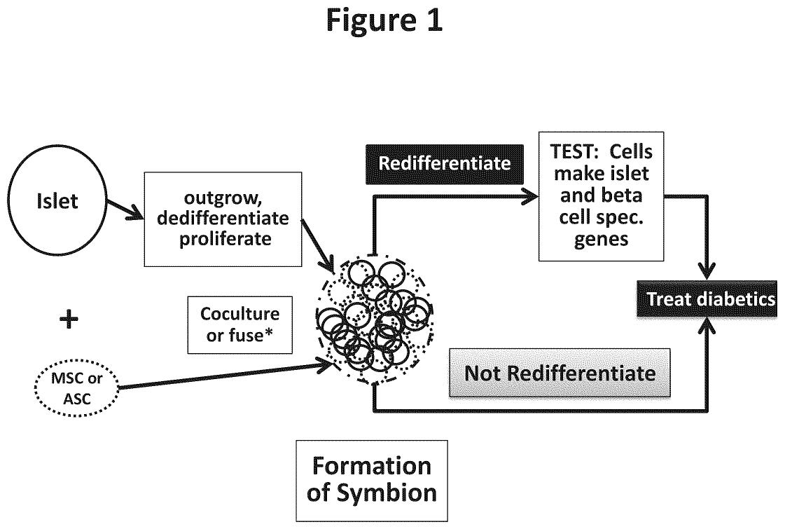

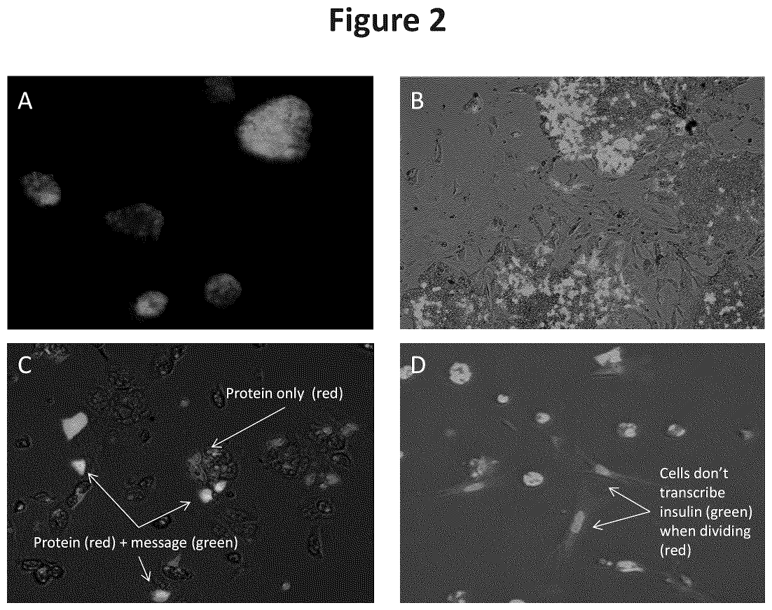

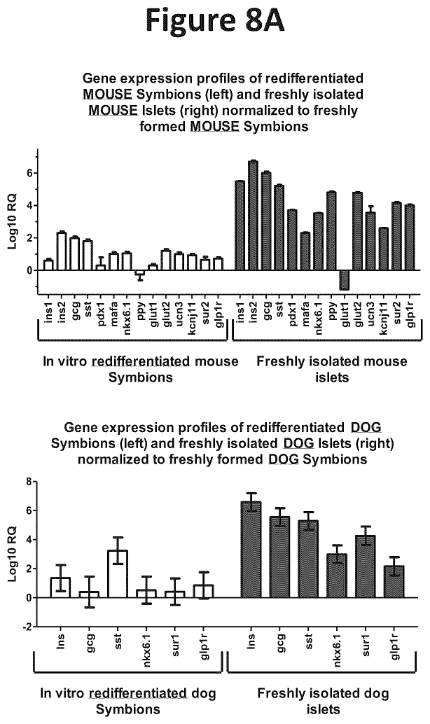

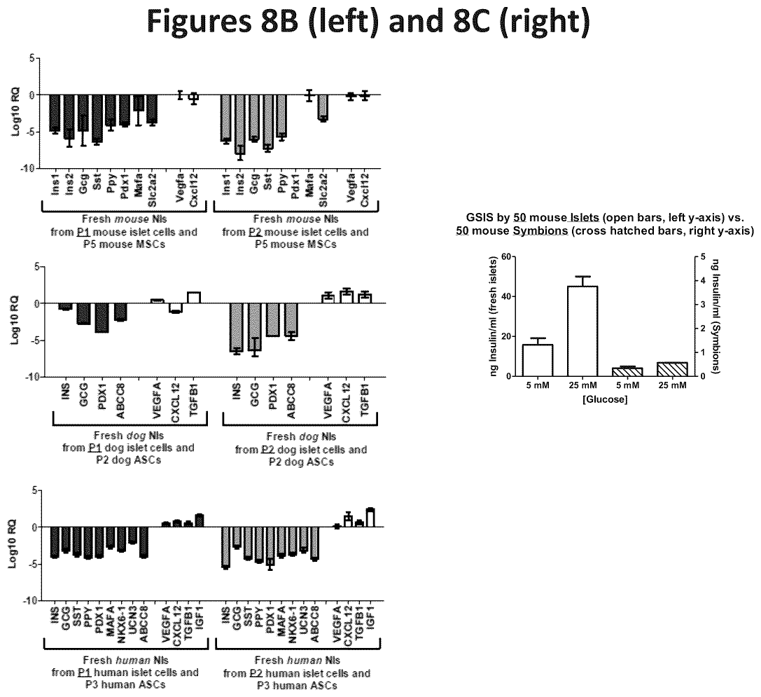

Outgrowth of pancreatic islet cells: Islet cells were either (1) dissociated with trypsin and cells plated in Laminin-511 and/or Laminin-411 (20 μg/ml) pre-coated Tissue Culture (TC) wells or flasks, or (2) whole islets were plated in Laminin-511 and/or Laminin-411 coated TC wells. See FIG. 1. In both cases, cells were cultured and allowed to propagate in RPMI or other suitable growth medium supplemented with 20% Fetal Bovine Serum (FBS)+glutamine/penicillin/streptomycin (GPS)+Exendin 4 (Glp-1 at 10 nM for rodent cell cultures) until sub-confluence (all supplements are commercially available). This process takes approximately one to two weeks. Islet cells dedifferentiated within a matter of days, judging from immunohistochemistry (IHC) for insulin presence, Insulin Enzyme Linked Immunosorbent Assay (ELISA), Glucose Stimulated Insulin Release assays (GSIS), gene expression profiles (rtPCR), and from murine cell lines transgenic for Green Fluorescent Protein (gfp) under the control of the insulin 1 gene promoter.[1] See FIGS. 2 and 8A-8C.

Cell cluster formation: ASCs (P1 to P4) or MSCs (P1 to P5) and Islet cells (P1 to P2) were co-cultured at a 1:1 ratio in ultra-low attachment surface culture dishes (Corning, Kennebunk, ME) and allowed to form NIs overnight. Control ASC and Islet cell clusters were formed by the same method. Prior to their in vivo administration, samples of NIs were tested by rtPCR for expression of islet and MSC associated genes (see below).

Staining for confocal microscopy: ASCs or MSCs were stained with Cell Tracker Green (green), and passaged pancreatic islet cells were stained with Lipophilic Tracer DiI (red) by following the manufacturers' instructions. Post cell staining, NIs were formed, collected, fixed in 10% formalin, and their nuclei were stained with 4′,6-diamidino-2-phenylindole dihydrochloride (DAPI) prior to confocal microscopy.

Lipophilic Tracer DiR labeling of cell clusters was carried out following the manufacturer's instructions.

Redifferentiation of cell clusters: Re-differentiation of cell clusters was achieved in vitro using commercially available additives, in a two-step process. Step 1: cell clusters of rodent, canine or human origin were cultured for 6-8 days in serum free DMEM containing 5.6 mM D-glucose and supplemented with: (a) 1% BSA fraction V, (b) ITS-G, (c) GPS. Step 2: After 6-8 days, this medium was replaced with Redifferentiation Medium (RDM) and cultured 2 weeks. RDM is DMEM containing 25 mM glucose and supplemented with: (a) N2 supplement A (commercially available), (b) SM-1 supplement (commercially available), (c) 10 mM Nicotinamide (commercially available), (d) 10 nM exendin 4 (commercially available), (e) 2 nM Activin A (commercially available). Redifferentiation tested and confirmed by rtPCR for expression of islet and MSC associated genes as described below.

Cell cluster cellular ratio assessment: For each species (mouse, dog, human), adherent cultures of ASCs and ICs were harvested as described above. ASCs were stained green with cell tracker green in order to be able to distinguish them from ICs. Staining efficiency was assessed by FACS and determined to be >95%. ICs were left unstained. NIs were formed overnight in six-well ultra-low adhesion plates as described above using 0.5×10⁶ ASCs and 0.5×10⁶ ICs per well in 2 ml DMEM/F12+10% FBS. The next day, NIs were collected and dissociated to single cell preparations by 30 minutes incubation with 1 ml Accumax per well. Single cell preparations were then resuspended in 1×PBS+1% BSA and analyzed by FACS (BD FACScan Analyzer, San Jose, California) for percent green (ASC) vs. unstained (IC) cells.

rtPCR: RNA was extracted from 1×10⁶ cell samples using a Qiagen RNeasy Mini Kit, including a DNase digestion step to exclude contaminating DNA, and following the manufacturer's instructions. Reverse transcription was performed using SuperScript II Reverse Transcriptase for 60 minutes at 42° C. Real-time PCR was carried out in duplicate using species-specific TaqMan primers (Applied Biosystems, ABS, Foster City, California) and following the manufacturer's instructions. All reactions were carried out in a total volume of 20 μL with TaqMan Universal Master Mix II with UNG. Reaction conditions were 50° C. for 2 minutes, followed by a 95° C. for 10 minutes start, and 40 cycles of melting at 95° C. for 15 seconds and annealing at 60° C. for 1 minute. All samples were run in duplicate, and the average threshold cycle (Ct) value was used for calculations. The ABS 7500 Real Time PCR System was used to monitor real-time PCR. Relative quantitation (RQ, normalization) of each target gene was calculated with the Ct method using the ABS software provided with the instrument, and by normalization to two internal housekeeping genes, beta actin and beta 2 microglobulin (B2m). RQ was calculated through normalization to external controls as indicated, and by using the software provided with the machine. Results are presented as log 10 (RQ)±log 10 (RQmin and RQmax). Differences greater than log 10 (RQ) 2 or less than log 10 (RQ)-2 were considered significant. Utilized PCR primers are listed in the following table.

| Target genes (MOUSE) | ABS catalog # |
|---|---|
| Actb | Mm04394036_g1 |
| B2m | Mm00437762_m1 |
| Ins1 | Mm01259683_g1 |
| Ins2 | Mm00731595_gH |
| Gcg | Mm01269055_ml |
| Sst | Mm00436671_ml |
| Ppy | Mm01250509_g1 |
| Pdx1 | Mm00435565_m1 |
| Mafa | Mm00845206_s1 |
| Slc2a1 | Mm00441480_m1 |
| Slc2a2 | Mm00446229_m1 |
| Ucn3 | Mm00453206_s1 |
| Abcc8 | Mm00803450_m1 |
| Nkx6-1 | Mm00454961_m1 |
| Glplr | Mm00445292_m1 |
| Kcnj11 | Mm00440050_s1 |
| Vegfa | Mm01281449_m1 |
| Cxcl12 | Mm00445553_m1 |
| Tgfb1 | Mm01178820_m1 |
| Igf1 | Mm00439560_m1 |
| Target genes (DOG) | ABS catalog # |
| ACTB | Cf03023880_g1 |
| B2M | Cf02659077_m1 |
| INS | Cf02647520_m1 |
| GCG | Cf02624195_ml |
| SST | Cf02625293_m1 |
| PDX1 | Cf02622671_m1 |
| NKX6-1 | Cf02705682_mH |
| ABCC8 | Cf02690717_m1 |
| GLPIR | Cf02696492_m1 |
| VEGFA | Cf02623449_ml |
| CXCL12 | Cf02625258_m1 |
| TGFB1 | Cf02623325_m1 |
| IGF1 | Cf02627846_m1 |
| IDO-1 | Cf02640742_m1 |
| Target genes (HUMAN) | ABS catalog # |
| ACTB | Hs01060665_g1 |
| B2M | Hs00984230_m1 |
| INS | Hs02741908_m1 |
| GCG | Hs01031536_m1 |
| SST | Hs00356144_m1 |
| PPY | Hs00358111_g1 |
| PDX1 | Hs00236830_m1 |
| MAFA | Hs01651425_s1 |
| NKX6-1 | Hs00232355_m1 |
| UCN3 | Hs00846499_s1 |
| ABCC8 | Hs01093761_ml |
| VEGFA | Hs00900055_m1 |
| CXCL12 | Hs03676656_mH |
| TGFB1 | Hs00998133_m1 |
| IGF1 | Hs01547656_m1 |

### Results

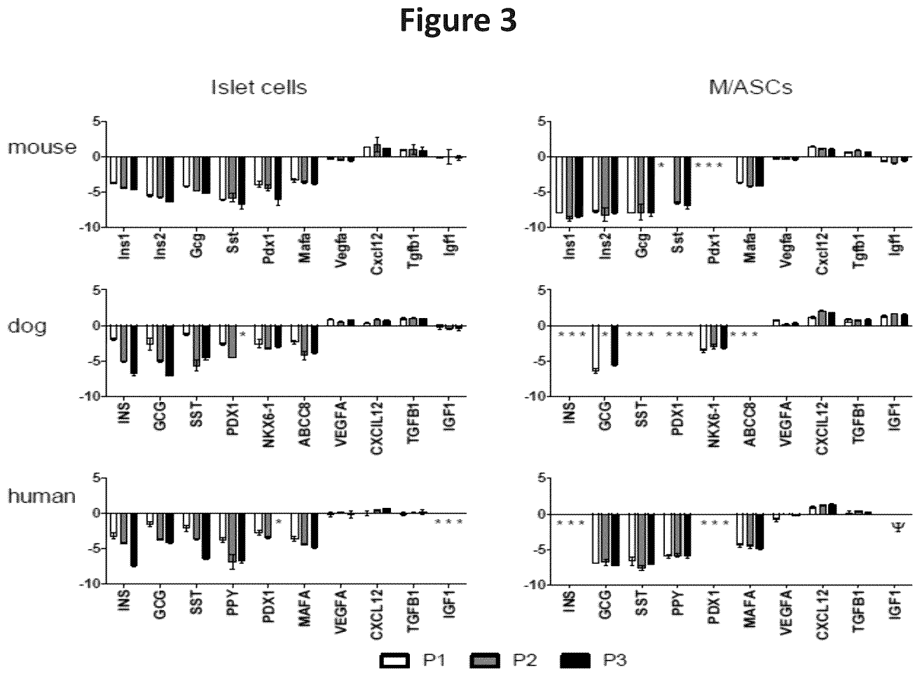

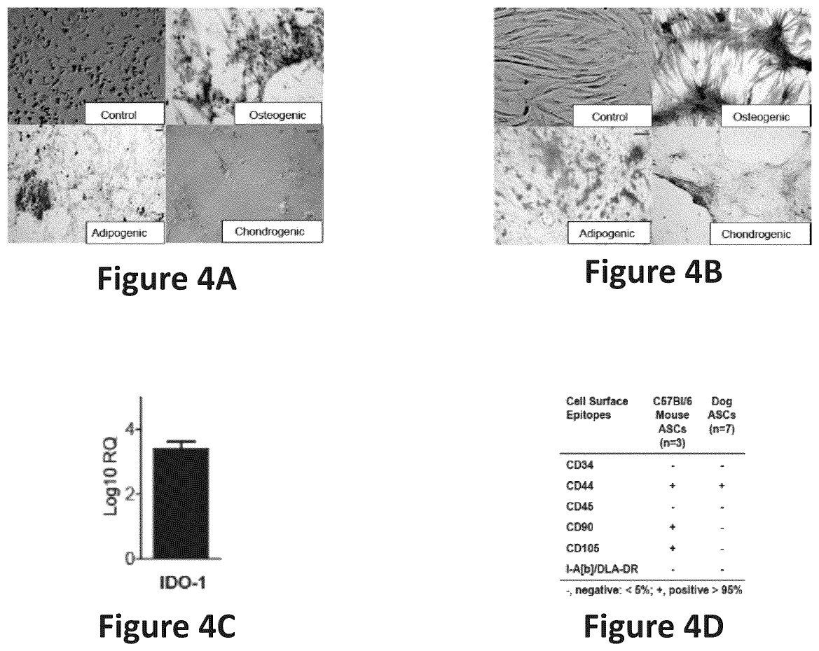

Growth and Characterization of ICs and M/ASCs: The NIs' starting materials, i.e., cultured ICs and M/ASCs, were obtained as follows. Freshly isolated islets from non-diabetic mice, dogs and humans were tested for viability, placed in culture, and grown and passaged as described in the above examples. ICs grow out of the islets, proliferate and dedifferentiate as they undergo EMT, a reversible process. Cultured ICs retain residual IC-associated gene expression profiles that decrease with passaging, and exhibit a gene expression pattern distinct from those of M/ASCs (FIG. 3). All ICs were used at P1-P2 for NI formation and experimentation. MSCs and ASCs were obtained from non-diabetic mice, dogs and humans and cultured and characterized as described in Example 3. All MSCs and ASCs met the minimal criteria of plastic adherence, ability to undergo trilineage differentiation, expression of characteristic cell surface epitopes, and importantly, absent expression of I-A[b] (mouse)/DLA-DR (dog)/HLA-DR (human) transplant antigens. Exposure of canine ASCs to IFNγ significantly induced indoleamine 2, 3 dioxygenase (IDO-1) gene expression (FIGS. 4A-4D), an important inhibitor of the T cell response in inflammatory states such as insulitis. M/ASCs were used at P1-P5 for NI formation and experimentation. Both ICs and M/ASCs were karyotyped (Veterinary Medicine and Biomedical Sciences, Texas A&M University, College Station, TX) and found to be normal.

Cell cluster formation and imaging: FIG. 1 shows a schematic of cell cluster formation and a proposed use. As shown in the figure, dedifferentiated pancreatic islet cells and ASCs or MSCs were used to form cell clusters that can be induced to produce islet cell specific proteins to treat T1DM or T2DM; dedifferentiated pancreatic islet cells and ASCs or MSCs. The pancreatic islet cells were first outgrown from the islet (mouse, canine or human), and allowed to dedifferentiate and proliferate for one or more passages. Once dedifferentiated, such cells express and produce significantly reduced to no islet cell specific genes or proteins, respectively. ASCs or MSCs were cultured by standard methods up to 4 passages. Once sufficient numbers of each cell type are available, the two cell types can be co-cultured in low-adhesion flasks to form islet-sized cell clusters.

FIG. 2 illustrates the outgrowth and Epithelial to Mesenchymal transition that resulted from culturing pancreatic islet cells in the manner described herein. To help illustrate this phenomenon, the transgenic, C57Bl/6, ins1gfp+ mouse, wherein the green fluorescent protein (gfp) is under the control of the Insulin 1 (ins1) gene promoter, was used.[1] As only islet beta cells express the Insulin 1 gene, insulin-gene-expressing beta cells isolated from this strain appear green, and are thus readily identifiable. Panel A shows whole islets isolated from the ins1-gfp+ mouse. These islets were cultured on Laminin-511 coated plates as described above. Panel B of FIG. 2 is of Ins1gfp+ whole islets after 6 days of culture. While there was still significant insulin gene expression where islet cells attached (green cells), cells are detaching from the islets and proliferating, and in these cells, insulin gene expression is downregulated (cells are no longer green). This is more fully illustrated in Panel C of FIG. 2, which depicts Ins1gfp+ pancreatic islet cells that were trypsinized to dissociate the islets prior to culture, then fixed and stained for Insulin protein (red). Where cells are green or yellow the ins1 gene is still actively transcribed and translated. Where cells appear red only, insulin protein is present, but lack of green (or yellow) color indicates the gene is down-regulated. Finally, Panel D of FIG. 2 shows Ins1gfp+ pancreatic islet cells that were grown in the presence of EdU to track cell division. These cells were fixed and stained with Hoechst (nuclei, blue) and for EdU (red). Cells in which ins1 gene translation is occurring appear green. Nuclei of cells that are dividing appear red. As can be seen in the image, cells that are not dividing are bright green and have a round, epithelial morphology, while cells that are dividing (red nuclei) are taking on an elongated, mesenchymal appearance, and are only faintly green, indicating the down-regulation of insulin gene expression.

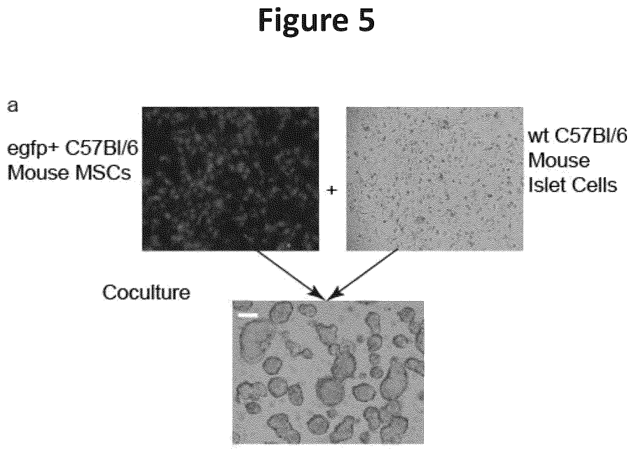

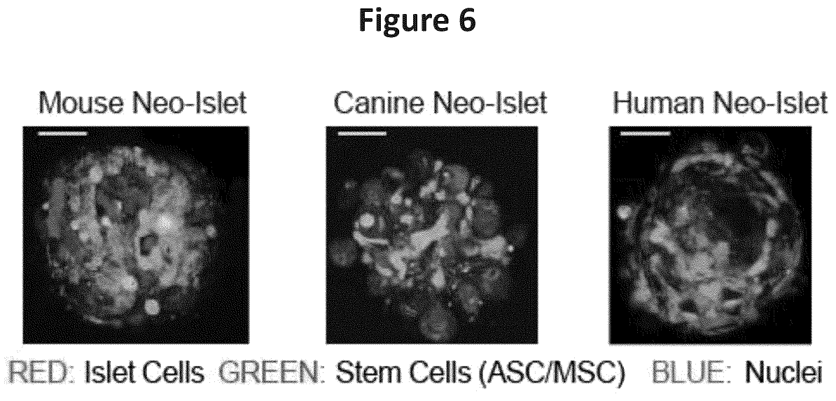

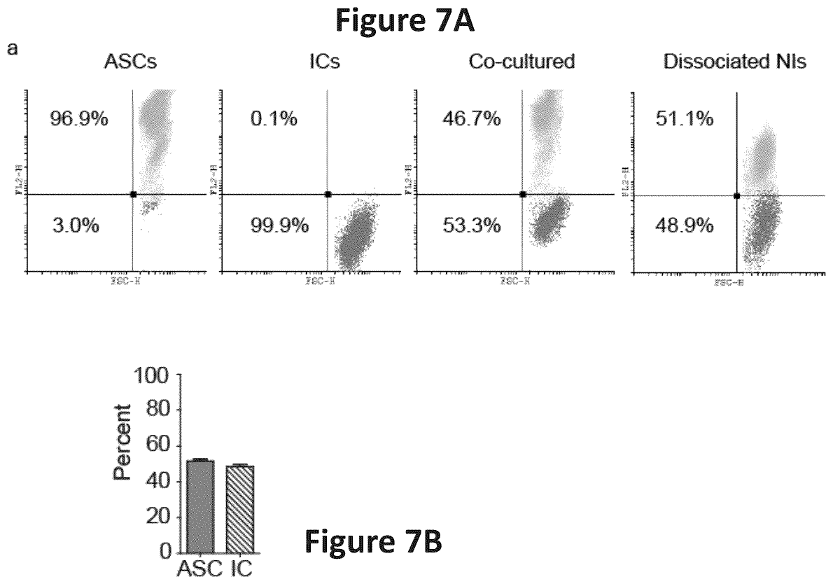

NIs of approximate islet size were prepared by overnight co-culturing of bone marrow-derived MSCs or their adipose-derived analogs ASCs (M/ASCs) with culture expanded murine pancreatic islet cells (ICs) at a 1:1 ratio (found to be optimal) in an ultralow cell adhesion system. An example of this process using mouse cells is shown in FIG. 5. To further assess the potential translational relevance of such murine NIs, we confirmed that comparable NIs could be readily generated from both canine and human ICs and M/ASCs. Green fluorescent protein positive (gfp+) C57Bl/6 mouse MSCs and C57Bl/6 mouse pancreatic islet cells were grown. The two cell types were then cultured in low-adhesion plates and formed cell clusters. Confocal images (63× magnification) of single Murine, Canine and Human cell clusters of ASCs (green) and pancreatic islet cells (red) are shown respectively in the left, middle and right images of FIG. 6. As can be seen, for cell clusters of either murine, canine or human origin, endocrine and stem cells are distributed equally throughout the cell cluster. The percent of each cell type in NIs was further assessed as follows. Canine ASCs were stained with Cell Tracker Green and cocultured with unstained pancreatic islet cells at a 1:1 ratio as above. After NIs were formed, they were dissociated with Accumax to single cells and analyzed by FACS as described in Online Methods, which revealed that at 24 hours post-formation, NIs are comprised of approximately 50% M/ASCs and 50% ICs (FIG. 7A), further indicating both cell types remain in a 1:1 ratio within NIs post-formation.

Gene expression profiles and Glucose Stimulated Insulin Secretion of murine, canine and human cell clusters: While these cell clusters do not express significant levels of insulin, as cultured pancreatic islet cells undergo an Epithelial to Mesenchymal transition when cultured, they may be redifferentiated in vitro using the redifferentiation protocol outlined above, such that they re-express islet cell genes. When the two cell types, ICs and M/ASCs, were combined to form NIs as shown in FIG. 5, the NI gene expression pattern exhibited characteristics of both cell types. FIG. 8A shows the islet gene expression profiles of mouse (top) and dog (bottom) cell clusters 14 days post exposure to redifferentiation medium (left sides) compared with those of freshly isolated mouse or dog islets (right sides). Both sets of gene expression profiles were normalized to those of freshly formed, dedifferentiated mouse (top) or dog (bottom) cell clusters. These results indicate that the freshly formed mouse and dog cell clusters express low levels of all tested islet associated genes, and have the capacity to undergo redifferentiation to express higher levels of these genes. Freshly formed NIs were able to secrete insulin in response to glucose in vitro, albeit at approximately 100-fold less than intact islets FIG. 8B shows the gene expression profiles of freshly formed murine (top), canine (middle) and human (bottom) cell clusters made from MSCs or ASCs and either P1 (left) or P2 (right) pancreatic islet cells as compared to freshly isolated islets from those species (normalization). As can be seen in this panel, freshly formed mouse, dog and human cell clusters all express low levels of islet associated genes, as well as genes associated with ASCs/MSCs (vegf-α, cxcl12, tgfβ1 and igf1), and for each of these species, the expression of islet cell genes decreases with higher islet cell passage number. As shown in FIG. 8C, in response to exposure to 25 mM glucose, the Glucose Stimulated Insulin Secretion (GSIS) by 50 freshly formed C57Bl/6 mouse cell clusters (P1 pancreatic islet cells and P5 MSCs, cross hatched bars) is approximately 1% that of 50 freshly isolated C57Bl/6 mouse islets (open bars). This parallels the decrease in insulin gene expression seen in FIG. 8B.

Conclusion: Taken together, these results indicate (i) that cell clusters of cultured pancreatic islet cells and either MSCs or ASCs can be readily formed in vitro; (ii) that across species (mouse, dog, human), such cell clusters are similar in appearance and gene expression profiles, expressing low levels of islet associated genes; (iii) that across species (mouse, dog, human) such cell clusters are capable of being redifferentiated in vitro to re-express pancreatic endocrine associated genes. Furthermore, these results suggest that these cell clusters may be of therapeutic use in treating insulin dependent and non-insulin dependent diabetic humans or animals.

### Example 6: In Vivo, Dose Finding and Proof of Principle Studies in Spontaneously Diabetic NOD Mice and STZ-Diabetic NOD/SCID Mice Treated I.P. With Rodent, Dog, or Human Cell Clusters

### Animal Models

All studies involving animals were conducted in adherence to the NIH Guide for the Care and Use of Laboratory Animals, and were supervised and approved by an institutional veterinarian and member of the IACUC. Mice and rats were purchased from either Jackson Laboratory (Bar Harbor, ME) or Harlan (Haslett, MI), and were housed at constant temperature and humidity, with a 12:12-hour light-dark cycle in regular, shoebox type caging. Unless otherwise indicated, all mice and rats had unrestricted access to a standard diet and tap water. All mouse experiments were carried out using female C57Bl/6, female NOD or female NOD/SCID mice weighing between 15 and 35 g. All rat experiments were conducted on male Sprague-Dawley rats weighing between 538 and 650 g.

Polydextran Particle Omental Uptake Protocol

Four, 2-year-old Sprague-Dawley rats weighing between 538-650 g were anesthetized and treated i.p. with 5 ml polydextran particles (PDP; sterile sephadex G-25, particle size 87-510 μm) suspended 1:1 in Normal Saline. On Day 7 post administration, animals were sacrificed, and their omenta and other organs were harvested and examined for the presence of PDP.

Diabetes Models

Streptozotocin (STZ): Non-Obese Diabetic/Severe Combined Immunodeficiency (NOD/SCID) and C57Bl/6 mice were made diabetic with 3-5 i.p. doses (1 dose per day) of 50-75 mg/kg b.w. STZ, freshly dissolved in 20 mM citrate buffer, pH 4.5. Mice were considered to be diabetic when their non-fasting blood glucose levels were >300 mg/dL on 3 separate days.

Spontaneous: Female NOD mice develop T1DM spontaneously between 12-20 weeks of age. Mice were considered to be diabetic when their non-fasting blood glucose levels were >300 mg/dL on 3 separate days. Insulin treatment: Where indicated in Results and the figures, insulin was administered to diabetic animals via slow-release, sub-cutaneous insulin pellets (Linbits). Animals were anesthetized with isoflurane, and 1-3 Linbit pellets were inserted just under the skin following the manufacturer's instructions. Tail vein blood glucose concentrations were monitored for several days to ensure animals were neither hyper- nor hypoglycemic.

Blood Glucose Monitoring: In all animal studies, blood glucose concentrations were assessed twice per week via tail vein sampling, and using a OneTouch Ultra 2 glucometer (level of detection, 20-600 mg glucose/dL). Anesthesia: Animals were anesthetized with isoflurane, 1-5%, using an inhalation rodent anesthesia system (Euthanex, Palmer, PA). Rectal temperatures were maintained at 37° C. using a heated surgical waterbed (Euthanex, Palmer, PA).

Treatment of diabetic NOD mice with allogeneic NIs from C57Bl/6 mice: Diabetic NOD mice' blood glucose levels were normalized with Linbits, and NIs, composed of P5 eGFP+ MSCs and P1 pancreatic islet cells (2×10⁵ NIs/kg b.wt. suspended in 0.5 ml serum-free DMEM-F12 medium; N=6) or vehicle (0.5 ml serum-free DMEM-F12 medium; N=6) were sterilely administered i.p., using light isofluorane anesthesia on Day 20 post-Linbit administration. No subsequent exogenous insulin was given in either group. At 10 weeks post NI administration, mice were euthanized, and their omenta, livers, spleens, lungs, kidneys and pancreases were harvested and examined by fluorescence microscopy for the presence of eGFP+ NIs. Sera were also collected to test for an allo-IgG response to the cells that make up the NIs. As a positive control for this test, an additional group of 3 NOD mice was given Linbits and treated i.p. with 2×10⁵ freshly isolated, allogeneic islets/kg b.wt. suspended in 0.5 ml serum-free DMEM-F12. These mice were euthanized 14 days post-islet administration, and their sera harvested and examined as above.

STZ diabetic C57Bl/6 mouse treatment with syngeneic NIs, ASC-clusters or IC-clusters: Four groups of 10-week old, STZ-diabetic, blood glucose controlled (via Linbits) wt C57Bl/6 mice were administered i.p. (i) 0.5 ml vehicle (serum-free DMEM-F12; N=6), or 2×10⁵/kg b.wt. (ii) freshly formed NIs (P5 eGFP+ MSCs and P1 ICs; N=6), (iii) clusters composed of P1 ASCs only (N=5), or (iv) clusters composed of P1 ICs only (N=5). Mice were followed as indicated. Upon euthanization, omenta, pancreata, spleens, livers, lungs and kidneys were harvested and fluoroscopically examined for the presence of eGFP+ NIs. In addition, islet associated gene expression profiles were obtained in all omenta and pancreata.

Treatment of non-diabetic mice with mouse or canine NIs: Mouse NIs: Six groups of 2 to 4, 12-week old C57Bl/6 mice each were administered i.p. either (i) 2×10⁵/kg b.wt. freshly formed syngeneic NIs (P5 MSCs and P1 ICs) suspended in 0.5 ml serum-free DMEM-F12, or (ii) 0.5 ml serum-free DMEM-F12 (vehicle). Mice were followed for up to 12 weeks. Canine NIs: Two groups of 9-week old NOD/SCID mice were treated i.p. with (i) 2×10⁵/kg b.wt. freshly formed cNIs suspended in 0.5 ml DMEM/F12 (N=6) or (ii) 0.5 ml serum-free DMEM-F12 (vehicle; N=3). Mice were followed for 10 weeks.

Results

To test our central hypothesis in a clinically highly informative autoimmune TIDM model, we first examined whether the i.p. administration of in vitro generated allogeneic NIs could reestablish euglycemia in spontaneously diabetic NOD mice as a reflection of (i) their survival, (ii) the redifferentiation of pancreatic islet cells contained in the NIs into functional insulin-producing cells in vivo, and (iii) the MSC-mediated cyto-, allo- and auto-immune protection of the transplanted cell clusters.

Treatment of spontaneously diabetic NOD Mice with allogeneic NIs. Since others found that islet progenitor cells and dedifferentiated islet beta cells can differentiate into functional endocrine cells in vivo, we tested whether allogeneic murine NIs as described herein which were administered i.p. to spontaneously diabetic NOD mice, which develop a T-cell mediated, autoimmune form of T1DM, would reestablish euglycemia. This protocol was chosen because it closely resembles the most common clinical situation in which a patient with T1DM receives an allogeneic pancreas or islet transplant. To facilitate both in vivo tracking and post-mortem localization, administered NIs were dually labeled with DiR and composed of P5 MSCs derived from C57Bl/6 mice transgenic for the eGFP gene, constitutively expressed in all tissues, and P1 ICs from wild type C57Bl/6 mice (see FIG. 5). Others have demonstrated that normalization of blood glucose levels with insulin enhances the redifferentiation of pancreatic islet cells into insulin producing cells in vivo which simultaneously reduces the glucotoxic effects on the transplanted cells. Thus, to avoid potential glucotoxic effects on the transplanted NIs, and to stimulate endocrine redifferentiation of transplanted ICs, blood glucose levels of twelve spontaneously diabetic, female NOD mice were normalized with the subcutaneous administration of insulin-releasing pellets (Linbits), an effect that lasts for 30-40 days post-administration.

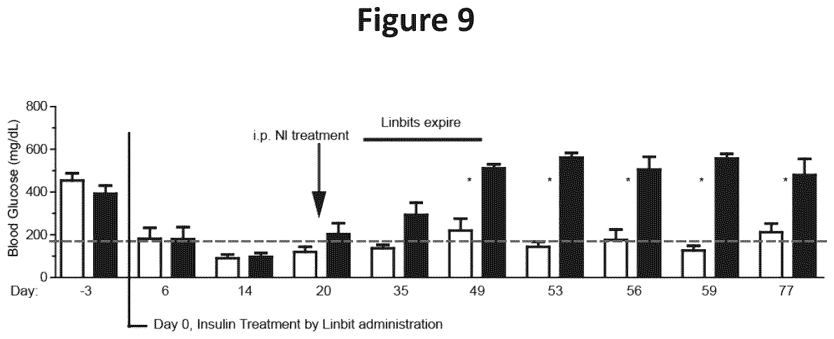

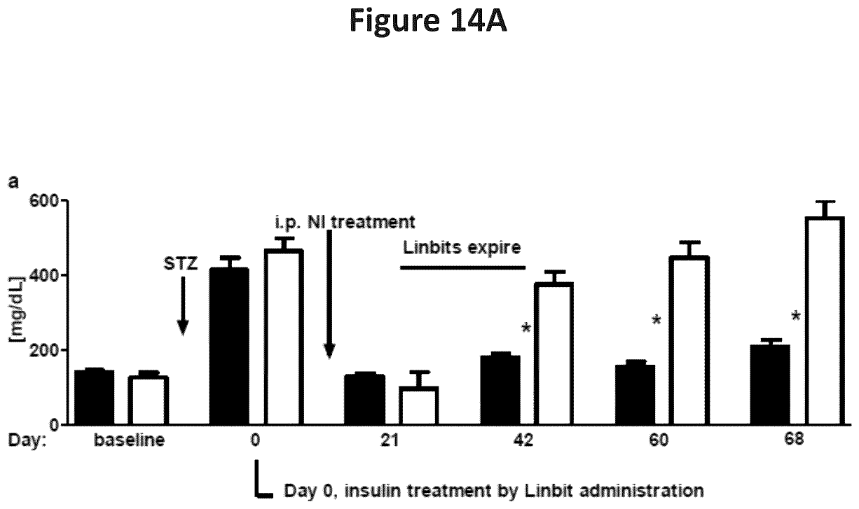

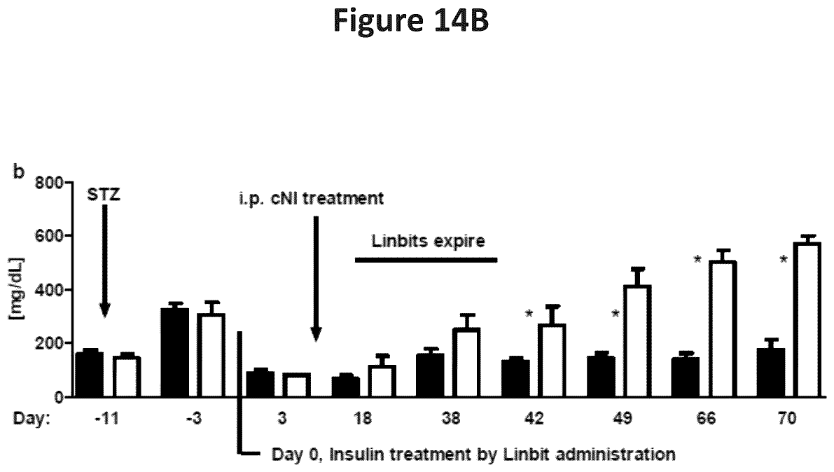

These mice were then divided into two groups and treated i.p. either with 2×10⁵/kg b.wt. NIs from allogeneic C57Bl/6 mice (N=6) or with vehicle (N=6; FIG. 9). As expected, by day 35-40 post-Linbit treatment, hyperglycemia redeveloped in vehicle-treated NOD mice, while strikingly, blood glucose levels in NI-treated animals remained near normal (FIG. 9) Similar restoration of normoglycemia was achieved in parallel experiments for Streptozotocin (STZ) diabetic C57Bl/6 mice, treated with syngeneic, and STZ-diabetic NOD/SCID mice, treated with xenogeneic (canine) NIs (FIGS. 14A and 14B).

Together, these data show that (i) the NIs engraft and survive, (ii) the ICs within the NIs redifferentiate in vivo, providing the mouse with a new, endogenous source of insulin, and (iii) the MSCs contained in the NIs effectively provide cyto-protection and allo- and auto-immune-isolation of the insulin producing cells in NOD mice, and apparently establishing glycemic control in in this clinically highly relevant T1DM model.

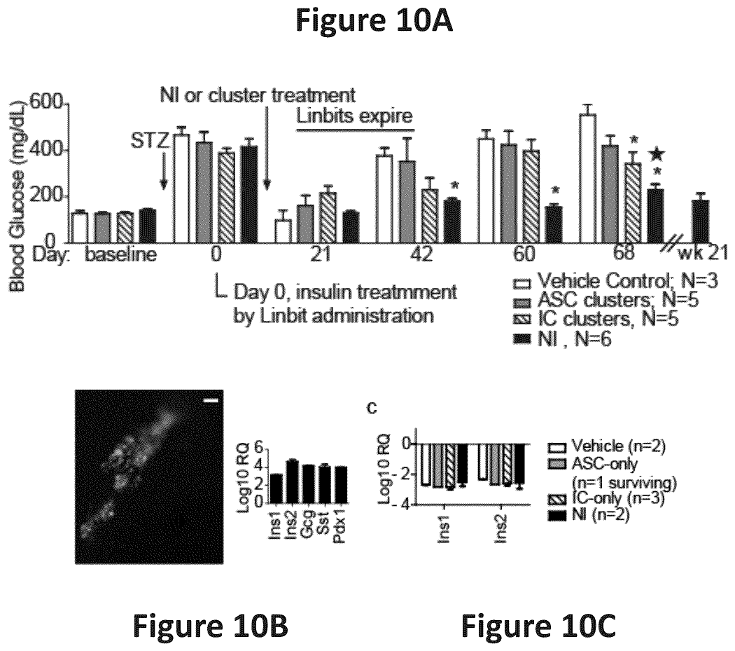

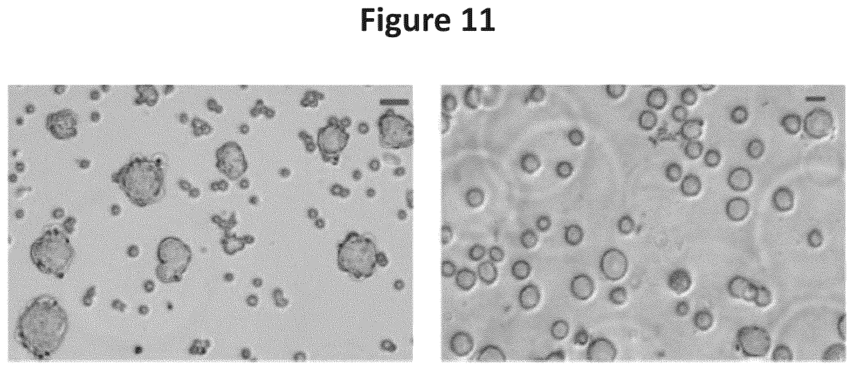

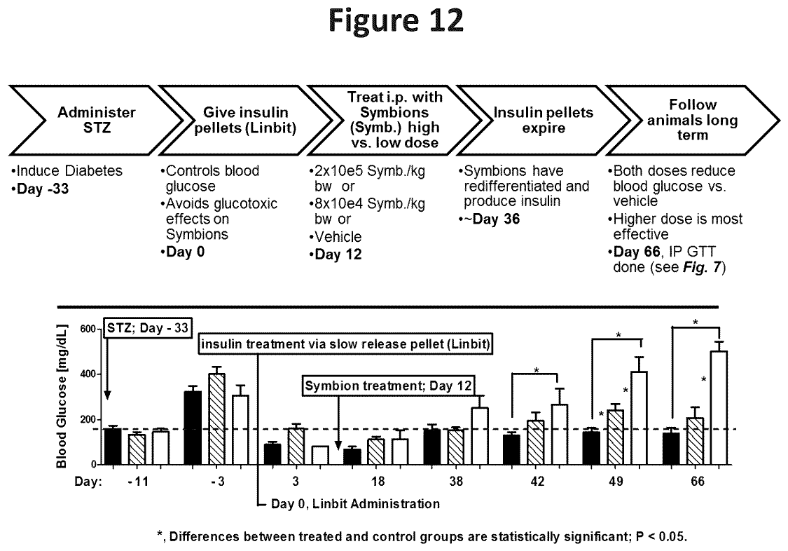

Collaboration of Islet Cells and M/ASCs within NIs is Essential to Establishing Normoglycemia in Diabetic Animals. To further explore the collaboration between ICs and M/ASCs in NIs, two experiments were conducted and are summarized in FIGS. 10A-10C. In these, a readily controllable Streptozotocin (STZ) model of T1DM in immune competent C57Bl/6 mice was used. In the first group, STZ-diabetic C57Bl/6 mice were treated i.p. with 2×10⁵/kg b.wt. syngeneic NIs or with vehicle. In the second, STZ-diabetic C57Bl/6 mice were treated i.p. with 2×10⁵/kg b.wt. control clusters composed of either ASCs (P1) or passaged ICs (P1) alone (FIG. 11). Importantly, the total number of cells in each generated cell cluster was identical to that in NIs (1,000 cells per cluster). Three mice from the NI-treated group, and all mice from the control groups were euthanized at 12 weeks. The remaining three NI-treated mice were followed for 21 weeks. Long-term (21 weeks) euglycemia was obtained in STZ-diabetic C57Bl/6 mice that were treated i.p. with syngeneic NIs. Significantly, treatment with identically generated control clusters composed of either ASCs or cultured ICs alone only minimally reduced blood glucose levels when IC clusters were given (FIG. 10A), demonstrating that both ICs and stem cells must be present within NIs to facilitate optimal redifferentiation of insulin producing cells.

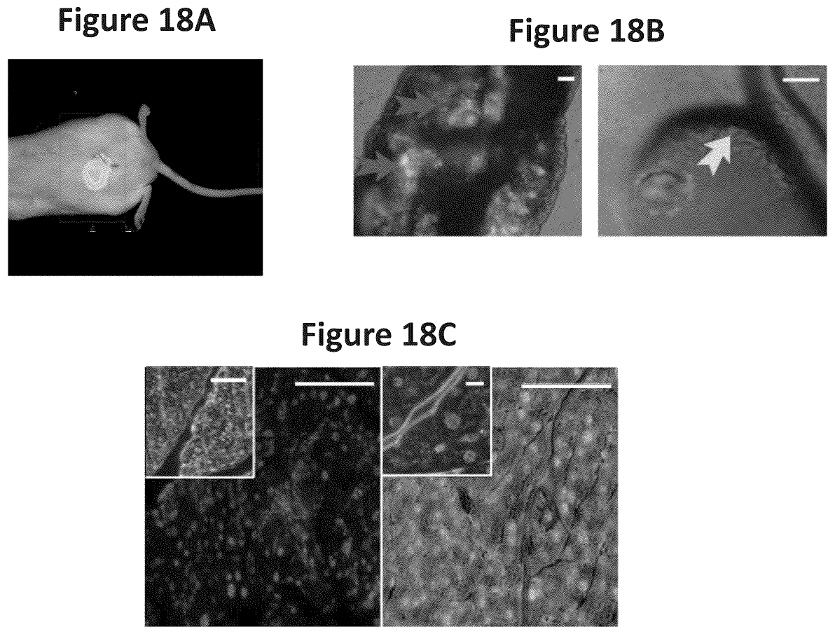

In vivo Redifferentiation. Data from the NOD mouse experiment (FIG. 9), as well as from retrieved omenta (FIG. 18B) imply that the NIs redifferentiate in vivo to produce sufficient insulin and other islet hormones to render mice euglycemic. Indeed, omenta retrieved from the euglycemic, C57Bl/6 NI-treated mice at 21 weeks showed both engraftment of NIs and significantly increased insulin, glucagon, somatostatin and PdxI gene expression compared to the NIs they were treated with (FIG. 10B). This clearly demonstrates effective in vivo redifferentiation of islet hormone-expressing pancreatic islet cells. Furthermore, expression of Ins1 and Ins2 in whole pancreata of STZ-diabetic mice was, as expected, significantly reduced in all animals (FIG. 10C), indicating that euglycemia in NI-treated mice was achieved by physiological insulin secretion provided by omentally engrafted NIs and not by residual pancreatic insulin.

### Example 7: In Vivo, Dose Finding and Proof of Principle Studies in STZ-Diabetic NOD/SCID Mice Treated I.P. With Canine Cell Clusters

Rationale: In Example 5, it was shown that freshly formed cell clusters of ASCs and dedifferentiated pancreatic islet cells express low levels of islet associated genes as well as ASC/MSC associated genes. It was also observed that the endocrine derived component of such cell clusters have the capacity to redifferentiate in vitro to re-express higher levels of islet associated genes. Others have shown that endocrine precursor cells can redifferentiate in vivo to produce insulin. We therefore tested (i) whether cell clusters comprising canine ASCs and dedifferentiated pancreatic islet cells can dose-dependently reverse hyperglycemia and affect animal survival, and (ii) whether removal of cell clusters would result in the return of hyperglycemia, confirming that cell clusters are exclusively responsible for the obtained treatment of T1DM.

Methods

cell clusters: cell clusters were formed from canine ASCs (passage 2) and canine cultured pancreatic islet cells (passage 1).

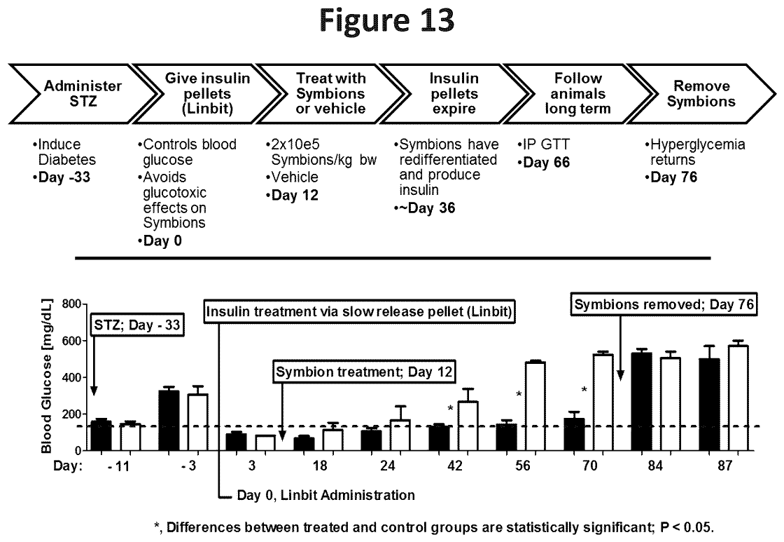

Diabetes Model: Non-obese diabetic/Severe Combined Immunodeficiency (NOD/SCID) mice were made diabetic with 5 i.p. doses of 50 mg/kg body weight Streptozotocin (STZ) in citrate buffer. Once blood glucose levels were >300 mg/dL on 3 separate days, they were given, on Day 0, one slow-release insulin pellet s.c. each (Linbit, Linshin, Canada) in order to control blood glucose levels and thereby avoid glucotoxic cell damage. These pellets expire by approximately 36 days (see FIG. 12). Animals were treated i.p. with (a) 200,000 or (b) 80,000 freshly formed, unredifferentiated canine derived cell clusters/kg body weight, or (c) vehicle (DMEM/F12). In some studies, NIs were surgically removed on day 76, and the mice were followed for an additional 2 weeks (see FIG. 13).

Intraperitoneal Glucose Tolerance Tests (GTT): At 55 days post treatment, 3 vehicle-treated and 5 canine cell cluster-treated mice were fasted 5 hours, whereupon baseline blood glucose levels were assessed using a OneTouch Ultra 2 Glucometer (Johnson and Johnson, New Brunswick, NJ; level of detection limit of 20 to 600 mg glucose/dL) Animals were then anesthetized, and 2 g glucose/kg bw (dissolved in serum free medium and filter sterilized) were administered via i.p. injection under isoflurane anesthesia. Blood glucose levels were assessed at 30 minutes, 60 minutes and 120 minutes post glucose administration.

Treatment Protocols

NOD allogeneic treatment: Once female NOD mice were confirmed to be hyperglycemic (non-fasting blood glucose >300 mg/dL on 3 separate days), they were treated s.c. with Linbit pellets. Once animals' blood glucose levels were normalized they were anesthetized (isoflurane), and cell clusters, composed of P5 gfp+ MSCs and P1 pancreatic islet cells (2×10⁵ cell clusters/kg bw suspended in 0.5 ml serum-free DMEM-F12 medium; n=6) or vehicle (0.5 ml serum-free DMEM-F12 medium; n=3), were administered i.p. No subsequent exogenous insulin was given to animals in either group. Blood glucose levels and body weights were assessed twice per week, and mice were followed for 10 weeks. At 10 weeks post cell cluster administration, animals were sacrificed, and their sera, omenta, livers, spleens, lungs and kidneys and pancreases were harvested, and examined for the presence of cell clusters and insulin. None were found anywhere but the omenta.

C57Bl/6 syngeneic treatment of STZ diabetic animals: STZ-diabetic, blood glucose controlled (via Linbits) C57Bl/6 mice were anesthetized and administered i.p. either (a) 2×10⁵ freshly formed gfp+ mouse cell clusters (P5 gfp+ MSCs and P1 pancreatic islet cells) stained with DiR and suspended in 0.5 ml serum free DMEM-F12 (vehicle; n=3), or (b) 2×10⁵ freshly formed gfp+ mouse cell clusters stained with DiR and embedded in Gelfoam (n=3). Control group animals were anesthetized and treated i.p. with 0.5 ml vehicle (n=3). Blood glucose levels and weights were assessed at baseline and then twice per week in all animals for 18 weeks. Once per week, the animals were examined under isofluorane anesthesia using a Licor, Pearl Impulse imager to track the cell clusters. Upon sacrifice, omentum, pancreas, spleen, liver, lungs and kidneys were harvested and examined for the presence of cell clusters. None were found anywhere but the omenta.

Treatment of non-diabetic mice: Mouse cell cluster administration: Six groups of 2 to 4 non-diabetic, 12 week old female C57Bl/6 mice (average weight of 21.9 g) each were anesthetized and administered i.p. either (a) 2×10⁵ freshly formed mouse cell clusters (P5 gfp+ MSCs and P1 pancreatic islet cells) suspended in 0.5 ml serum-free DMEM-F12 (5 groups sacrificed at different time points for tracking purposes), or (b) 0.5 ml serum free DMEM-F12 (vehicle; 1 group). Blood glucose levels and weights were assessed at baseline and then twice per week for up to 12 weeks. Canine cell cluster administration: Two groups of non-diabetic, 9 week old, female NOD/SCID mice weighing 19.7 to 24.8 g were anesthetized administered i.p. either (a) 2×10⁵ freshly formed, DiR stained canine cell clusters suspended in 0.5 ml DMEM/F12 (N=6) or (b) 0.5 ml serum free DMEM-F12 (vehicle; n=3). Blood glucose levels and weights were assessed at baseline and then twice per week for 10 weeks. Once per week, the animals were examined under isofluorane anesthesia using a Li-Cor, Pearl Impulse imager to track the cell clusters. Upon sacrifice, omentum, pancreas, spleen, liver, lungs and kidneys were harvested and examined for the presence of cell clusters. None were found anywhere but the omenta.

NOD/SCID recent onset diabetes, xenogeneic treatment: Groups of female, 20 week old, STZ-diabetic NOD/SCID mice weighing 17-29 g (n=5 per group) whose blood glucose levels were controlled with Linbit pellets were anesthetized and treated i.p. with (a) 2×10⁵ or (b) 8×10⁴ freshly formed, unredifferentiated canine cell clusters/kg bw embedded in Gelfoam, or (c) vehicle (DMEM/F12). Cell clusters were composed of P1 Islet cells and P2 canine ASCs. Blood glucose levels and body weights were assessed twice per week, and mice were followed for 13 weeks. IP GTTs were performed in the high dose group at 55 days post treatment, and cell clusters were surgically removed from the high dose group of mice in week 10.

NOD/SCID remote onset diabetes, xenogeneic treatment: 11 week old female NOD/SCID mice weighing 18.4 to 22.8 g were made diabetic with three i.p. doses of 75 mg/kg body weight STZ in citrate buffer. The diabetic state was confirmed by blood glucose levels of >300 mg/dL on 3 separate days. Once the animals were confirmed to be diabetic, their blood glucose levels were controlled for approximately 3 months with insulin therapy using s.c. linbit pellets. To confirm that all animals were still diabetic prior to cell cluster or vehicle administration, Linbits were allowed to expire, and mice to re-develop hyperglycemia. Mice were again treated with Linbits (Day 0) to control blood glucose levels and prevent glucotoxic cell cluster damage. Anesthetized mice were then treated i.p. with either (i) 2×10⁵ cell clusters/kg b.w. embedded in gelfoam or (ii) vehicle (0.5 ml DMEM/F12). Cell clusters were composed of P1 Islet cells and P2 canine ASCs. Blood glucose levels and body weights were assessed twice per week, and mice were followed for 11 weeks. IP GTTs: At 55 days post treatment, 3 vehicle-treated and 5 cell cluster-treated mice were fasted 5 hours, whereupon baseline blood glucose levels were assessed. Animals were then anesthetized, and 2 g glucose/kg bw (dissolved in 0.5 ml serum free medium and filter sterilized) were administered via i.p. injection under anesthesia. Tail vein blood glucose levels were assessed at 30 minutes, 60 minutes and 120 minutes post glucose administration.

ELISA for Canine Insulin: Sera from vehicle and cell cluster-treated mice that had been collected during the glucose tolerance tests were examined by ELISA for the presence of canine specific insulin that does not cross react with mouse insulin (Mercodia, Uppsala, Sweden), following the manufacturer's instructions. Sera from a dog, as well as from a C56Bl/6 mouse were also analyzed as positive and negative controls, respectively, for cross-reactivity.

Antibody Response Test: Aliquots of ˜5×10⁴ cells (MSCs, ASCs or pancreatic islet cells that were used to create the cell clusters that were administered) were each incubated with ˜500 μl of serum obtained from cell cluster or canine ASC-treated NOD mice >14 days post cell cluster or ASC administration. The cells were incubated with the sera for 30 minutes at room temperature. After 30 minutes cells were centrifuged at 600×g for 5 minutes, resuspended in FACS buffer and incubated with cy3-conjugated goat-anti-mouse (dilution) IgG antibody. The cells were incubated an additional 20 minutes in the dark at room temperature. One ml 1×PBS+1% BSA was then added, the cells vortexed, centrifuged, resuspended in 400 μl fixation buffer (1% Formaldehyde), and analyzed by FACS (BD FACScan Analyzer, San Jose, CA). A shift of >7% of the cells was considered a positive response, indicating that the serum contained antibodies to the tested cells. Embedding cell clusters in Gelfoam: Individual doses of cell clusters were collected in a 15 ml Falcon tube and centrifuge at 200×g for 2 minutes. The supernatants were discarded, and the pellets resuspended in 0.2 ml serum-free DMEM-F12 each. The cell cluster suspensions were then loaded into 0.5×0.5×0.5 cm blocks of sterile Gelfoam, which were incubated in a 37° C. incubator for 3 hours prior to i.p. administration to mice. Cell cluster embedded in Gelfoam were surgically transplanted under sterile conditions and under anesthesia onto the peritoneal fat-pads and omenta of recipient mice. The abdominal incision was closed with two layer sutures.

In vivo Imaging: In vivo imaging of DiR stained cell clusters was performed in anesthetized mice using the Li-Cor, Pearl Impulse imager.

Results

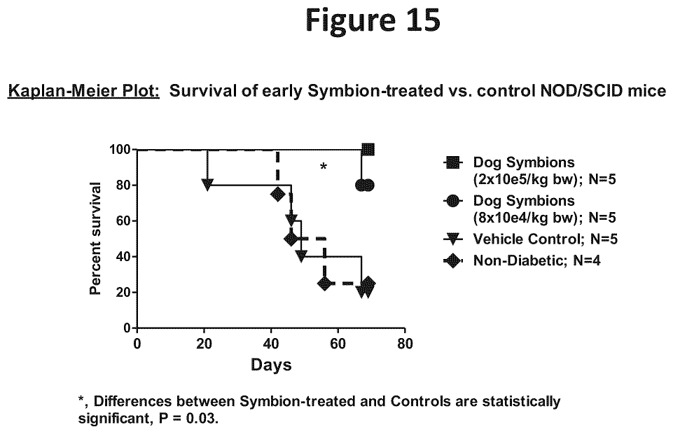

A dose of 2×10⁵ cell clusters/kg bw administered i.p. 1 month post STZ achieves and maintains euglycemia and promotes animal survival: Three groups of five NOD/SCID mice each were treated i.p. approximately one month after establishment of STZ-induced T1DM diabetes with (a) 200,000 or (b) 80,000 freshly formed, unredifferentiated canine derived cell clusters/kg body weight, suspended in 0.5 ml serum free medium (DMEM-F12), or (c) vehicle (0.5 ml serum free medium). Linbits were given once, ˜1 month after STZ administration (Day 0 in FIGS. 12, 13, 14A, and 14B), and cell clusters or Vehicle were administered once blood glucose levels were stabilized, 12 days post Linbit administration. Vehicle-treated animals began to die by day 21, despite insulin therapy (see FIG. 15). As shown in FIGS. 12, 13, and 14B, once the Linbits wore off, remaining animals treated with vehicle (open bars) again became hyperglycemic. Cell cluster-treated diabetic animals (black and cross hatched bars) showed normalized blood glucose levels, with the 200,000 cell clusters/kg bw dose more effectively controlling hyperglycemia than the 80,000 cell clusters/kg bw dose. As FIG. 15 demonstrates, mortality rates were significantly lower in the treated groups (squares and circles) than in either the vehicle-treated diabetic group (triangles) or, surprisingly, the healthy control, non-diabetic group (diamonds).

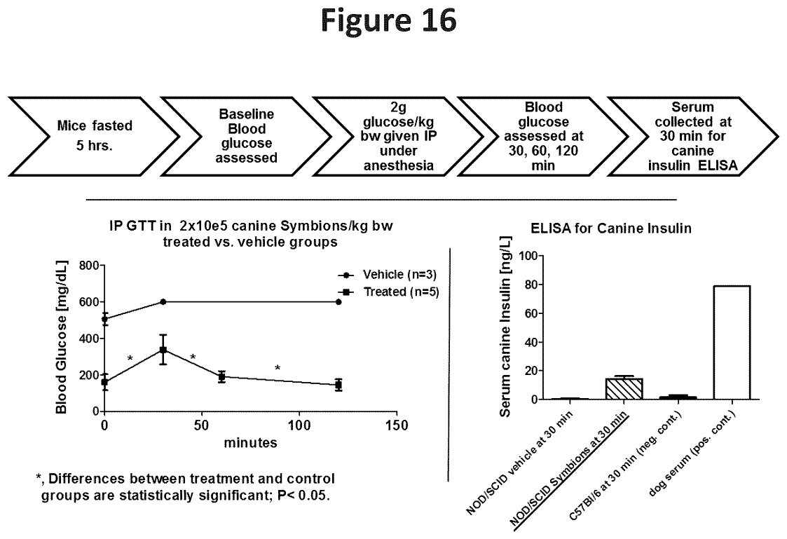

Intraperitoneal glucose tolerance tests (IP GTTs) were normal in 2×10⁵ cell clusters/kg bw-treated animals, and a rise in blood glucose was accompanied by release of canine insulin: IP GTTs (2 g glucose/kg bw) were performed at 54 days post canine cell cluster treatment (66 days post Linbit therapy) on NOD/SCID mice that had been treated with either the 2×10⁵ canine cell clusters/kg body weight dose or vehicle as described in the Methods. As seen in FIG. 16, IP GTTs of cell cluster-treated animals were normal, whereas blood glucose levels of vehicle-treated mice remained elevated 2 hours post glucose administration.

Sera from vehicle and cell cluster-treated mice that had been collected during the glucose tolerance tests were examined by ELISA for the presence of canine specific insulin that does not cross react with mouse insulin as described in Methods. As can be seen in FIG. 16, canine insulin was detected in the sera of cell cluster-treated (cross hatched bar), but not vehicle-treated mice. The sera of healthy C57Bl/6 mice were tested as well to ensure there was no significant cross reactivity between canine and mouse insulin. No significant cross-reactivity was observed.

Retrieval of canine cell clusters reestablishes hyperglycemia: On Day 76, the cell clusters were removed from the 2×10⁵ cell clusters/kg bw treatment group. As FIG. 13 demonstrates, removal of canine cell clusters resulted in reestablishment of hyperglycemia in this group of animals (black bars) similar to that of vehicle-treated animals (open bars).

Conclusion: The results presented in Example 6 demonstrate that freshly formed canine cell clusters administered i.p. to recent onset diabetic animals redifferentiate in vivo to provide adequate and physiologic insulin secretion and durable, but reversible, maintenance of euglycemia in rodents with T1DM. In addition the ability to remove the clusters via removal of the omentum is a safety feature of this technology when clinically warranted.

### Example 8: Cell Cluster Tracking and Angiogenesis

Rationale: (A) It is well known that the Omentum accumulates cells and foreign bodies of various sizes. Thus we hypothesized and tested whether the cell clusters when delivered i.p. would be taken up by and engraft in the Omentum. Such a location offers two advantages: (i) As is the case for the pancreas, blood from the Omentum drains directly into the liver via the portal system. Thus insulin and other islet hormones made by the cell clusters would be delivered in physiological fashion. (ii) The Omentum can be removed without significant ill effects, should it be desired for safety or other reasons that the cell clusters be removed. (B) As MSCs and ASCs express potent angiogenic and survival factors, we also examined whether the stem cell component of the engrafted cell clusters enhanced the development of a blood supply for the cell clusters.

Methods

Mouse cell clusters were generated from co-culture in low adherence vessels of P2 Islet cells derived from wild-type C57Bl/6 mice and P5 MSCs derived from C57Bl/6 mice transgenic for the GFP⁺ gene to facilitate tracking of the cell clusters in vivo. As indicated below, in one group of experiments, after formation, the cell clusters were stained with the Infrared light-excitable carbocyanine probe DiR (Molecular Probes, Eugene, OR) to allow for tracking in vivo.

Dog cell clusters were formed from co-culture in low adherence vessels of P2 dog pancreatic islet cells and P4 dog ASCs that had been stained with DiR to allow for tracking in live animals.

Diabetes model and allogeneic treatment: Female Non-Obese Diabetic (NOD) mice spontaneously develop T1DM at approximately 12-20 weeks of age. Once female NOD mice were confirmed to be hyperglycemic (blood glucose >300 mg/dL on three separate days), they were treated s.c. with Linbit pellets. Once animals' blood glucose levels were normalized, cell clusters (2×10⁵ cell cluster/kg bw suspended in 0.5 ml serum free DMEM-F12 medium) or vehicle (0.5 ml serum free DMEM-F12 medium) were administered i.p. to groups of five animals each. No subsequent exogenous insulin was given to animals in either group. Blood glucose levels and body weights were assessed twice per week, and mice were followed for 10 weeks. At 10 weeks post cell cluster administration, animals were sacrificed, and their sera, omenta, livers, spleens, kidneys and pancreases harvested.

Canine cell cluster administration: DiR labeled dog cell clusters were administered i.p. to 6 NOD/SCID mice, and the mice were examined weekly for 10 weeks under isoflurane anesthesia using a Li-Cor Pearl Impulse™ imager to track the cell clusters.

Syngeneic cell cluster administration: Two syngeneic administration experiments were performed, one in non-diabetic animals, and another in diabetic animals.
A) Non-diabetic animals: Six groups of non-diabetic C57Bl/6 mice were administered i.p. either (a) 2×10⁵ freshly formed gfp+ mouse cell clusters suspended in 0.5 ml serum free DMEM-F12 (5 groups), or (b) 0.5 ml serum free DMEM-F12 (vehicle; 1 group). Blood glucose levels (OneTouch Ultra 2 glucometer) and weights were assessed at baseline and then twice per week in all animals. At various time points up to 12 weeks, groups of animals were sacrificed and their sera, omenta, livers, spleens, lungs, kidneys and pancreases harvested.
B) Diabetic animals: Two groups of STZ-diabetic C57Bl/6 mice were administered i.p. either (a) 2×10⁵ freshly formed gfp+ mouse cell clusters stained with DiR and suspended in 0.5 ml serum free DMEM-F12, or (b) 0.5 ml serum free DMEM-F12 (vehicle). Blood glucose levels (OneTouch Ultra™ 2 glucometer) and weights were assessed at baseline and then twice per week in all animals for 13 weeks. Once per week, the animals were examined under isoflurane anesthesia using a Licor, Pearl Impulse imager to track the cell clusters.

Immunohistochemistry: Omenta and other organs were harvested, fixed and embedded as previously described.[18] Omental sections were deparaffinized and stained by immunohistochemistry for DNA with 4′,6-diamidino-2-phenylindole (DAPI, Molecular Probes, Eugene, OR) and insulin protein using a guinea-pig anti-insulin antibody (Dako, Carpinteria, CA), and a cy³-conjugated anti-guinea pig antibody (Jackson ImmunoResearch, West Grove, PA) following the manufacturers' instructions.

Results

Cell clusters spontaneously engraft in the murine Omentum and produce Insulin. We hypothesized that injected NIs would home to, attach to, and engraft in the mice' well-vascularized omenta, which would offer the advantage of physiologic insulin secretion into the portal system of the liver. Indeed, as shown in FIG. 18A, fluorescence in vivo imaging of a euglycemic NOD mouse treated 10 weeks previously with DiR labeled NIs demonstrates their persistent location in the upper abdomen.

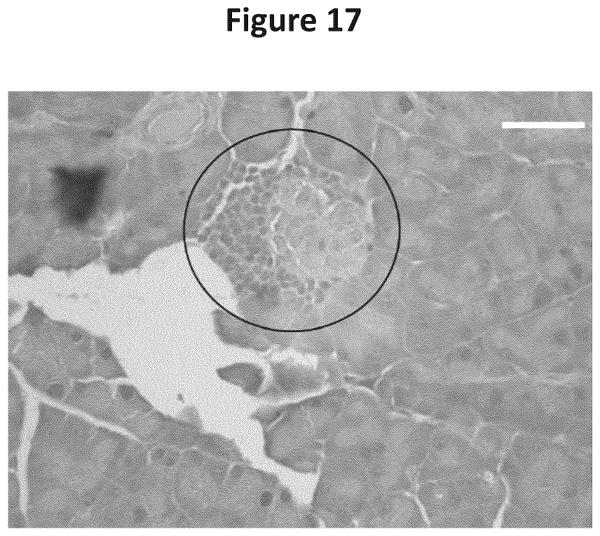

To further assess the intraperitoneal engraftment pattern and function of DiRlabeled, eGFP+ NIs as detected in FIG. 18A, upon euthanasia of the NI-treated NOD mice from the experiment shown in FIG. 9, we examined histologically the omenta, pancreata, spleens, livers, lungs and kidneys for the presence of eGFP+ NIs. NIs were detected only in the animals' omenta (FIG. 18B). Furthermore, sections of the omentum stained positive for insulin (FIG. 18C, left panel), while negative controls (FIG. 18C, inset) and omenta from vehicle treated, diabetic NOD mice showed no insulin staining (FIG. 18C, right panel). Pancreata were shown to have high-grade insulitis, as expected (FIG. 17), indicating that euglycemia was not achieved through islet recovery, but rather through physiologic insulin secretion by the omentally engrafted NIs. Importantly, there was no histologic evidence for tumor formation or ectopic maldifferentiation (adipo-, osteo-, chondrogenic) in any of the examined organs.

Conclusion: Taken together, the foregoing results demonstrate that across species: (i) cell clusters that are administered i.p. engraft in the omentum where they remain long term, redifferentiate, secrete insulin in physiologic fashion and are not rejected. (ii) The angiogenic properties of the stem cell component of the cell cluster helps vascularize the cell clusters, providing them with needed oxygen, nutrition, and optimized delivery of insulin from the cell clusters into the portal vein of the liver.

### Example 9: Cell Cluster Treatment of Remote Onset Diabetics

Rationale: We showed in Example 5 that the cell clusters are effective in treating recent onset T1DM. We tested here whether cell clusters were also effective in treating remote onset T1DM.

Methods

Cell clusters: cell clusters were formed from canine ASCs (passage 2) and canine cultured pancreatic islet cells (passage 1).

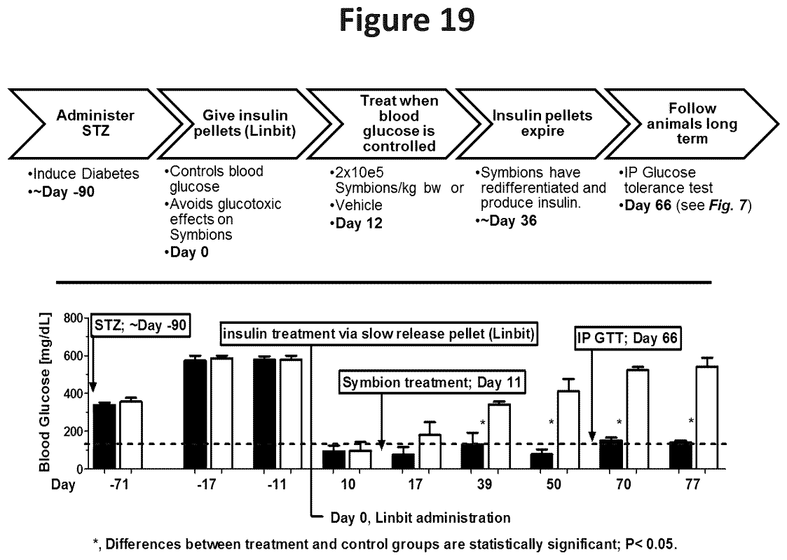

Diabetes Model: Non-obese diabetic/Severe Combined Immunodeficiency (NOD/SCID) mice were made diabetic with 3 i.p. doses of 75 mg/kg body weight Streptozotocin (STZ) in citrate buffer. The diabetic state was confirmed by blood glucose levels of >300 mg/dL on 3 separate days. Once the animals were confirmed to be diabetic, their blood glucose levels were controlled for approximately 3 months with insulin therapy using s.c. linbit pellets. To confirm that all animals were still diabetic prior to the cell cluster or vehicle administration, Linbits were allowed to expire, and all mice re-developed hyperglycemia. Mice were again treated with Linbits (Day 0 on FIG. 19 to control blood glucose levels and prevent glucotoxic cell damage.

IP GTTs and Insulin ELISAs—were carried out as described in Example 5, and results were combined with those of animals in Example 6 (recent onset) and presented in FIG. 16.

Results

Two groups of 5 diabetic NOD/SCID mice each were treated i.p. at 3 months after STZ-induced T1DM with (a) 200,000 freshly formed canine derived cell clusters/kg body weight suspend in 0.5 ml serum free medium (DMEM-F12) or (b) vehicle. An overview of the experimental design is given in FIG. 19. As shown in FIG. 19, animals with remote onset diabetes exhibit normoglycemia following treatment with canine cell clusters (black bars), while those treated with vehicle (open bars) remain hyperglycemic once their insulin pellets expire.

Conclusion: The above data demonstrate that, as is the case with recent onset diabetes, cell clusters are also effective in establishing euglycemia in remote onset diabetes.

### Example 10: Treatment of Spontaneously Diabetic NOD Mice with Allogeneic Mouse Cell Clusters

Rationale: We showed in Examples 5 and 7 that canine cell clusters can reverse STZ induced diabetes in NOD/SCID mice. While the NOD/SCID data presented above indicate that cell clusters generated from canine derived cells are capable of safely and effectively undergoing redifferentiation in vivo to produce insulin and secrete it in physiologic fashion long-term, the NOD/SCID model does not address the issues of protection of the transplanted cells from diabetogenic autoimmune and allo-immune attacks. ASCs and MSCs exhibit powerful immune modulating properties.[19] We hypothesized the stem cell component of the cell clusters would provide local immune isolation, and tested whether the cell clusters could restore euglycemia when administered allogenically to spontaneously diabetic NOD mice.

Methods

Mouse cell clusters were generated from co-culture in low adherence vessels of P2 Islet cells derived from wild-type C57Bl/6 mice and P5 MSCs derived from C57Bl/6 mice transgenic for the GFP⁺ gene.

Spontaneous diabetes model and allogeneic treatment: Once female NOD mice were confirmed to be hyperglycemic (blood glucose >300 mg/dL on 3 separate days), they were treated s.c. with Linbit pellets. Once animals' blood glucose levels were normalized, cell clusters (2×10⁵ cell cluster/kg bw suspended in 0.5 ml serum free DMEM-F12 medium) or vehicle (0.5 ml serum free DMEM-F12 medium) were administered i.p. to groups of 5 animals each. No subsequent exogenous insulin was given to animals in either group. Blood glucose levels and body weights were assessed twice per week, and mice were followed long term.

Results

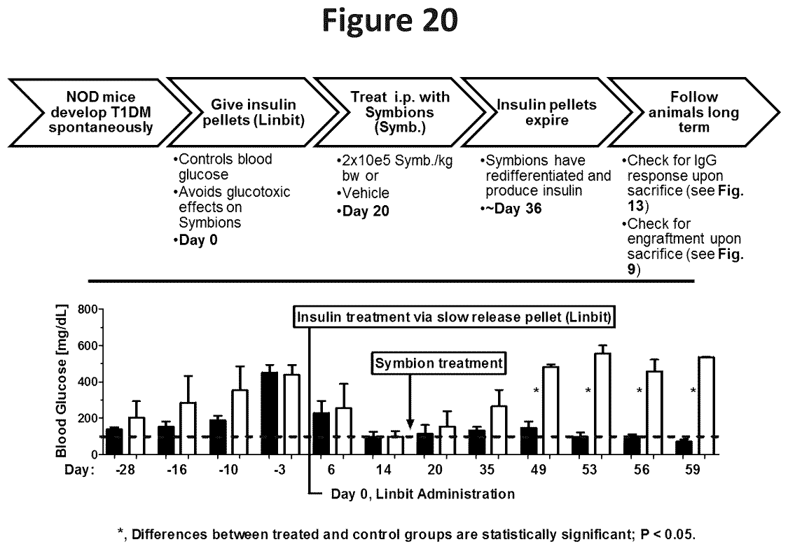

A dose of 200,000 allogeneic cell clusters/kg bw administered i.p. achieves and maintains euglycemia in spontaneously diabetic NOD mice. Blood glucose levels of vehicle and cell cluster-treated NOD mice are shown in FIG. 20. To summarize, blood glucose levels were normalized in mice treated with allogeneic cell clusters (black bars), while vehicle-treated mice (open bars) remained hyperglycemic.

Conclusion: These data demonstrate that, like the canine cell clusters, mouse cell clusters (i) redifferentiate in vivo to provide adequate insulin secretion to reestablish and maintain euglycemia, and importantly (ii) that they afford immune isolation against both allo- and auto-immune attacks without encapsulation, as hypothesized.

### Example 11: Cell Clusters do not Induce Hypoglycemia in Non-Diabetic Mice

Rationale: From the previous examples, it is apparent that the cell clusters engraft in the omentum where they redifferentiate to produce and secrete insulin. However, if insulin secretion were to be constitutive and non-physiologic, this could potentially lead to episodes of hypoglycemia. We tested, therefore, whether administration of cell clusters to non-diabetic animals would result in hypoglycemia.

Methods

Mouse cell clusters were generated from co-culture in low adherence vessels of P2 Islet cells derived from wild-type C57Bl/6 mice and P5 MSCs derived from C57Bl/6 mice transgenic for GFP⁺ gene.

Dog cell clusters were formed from co-culture in low adherence vessels of P2 dog pancreatic islet cells and P4 dog ASCs.

Mouse cell cluster administration: Six groups of 2 to 4 non-diabetic C57Bl/6 mice each were administered i.p. either (a) 2×10⁵ freshly formed mouse scell clusters suspended in 0.5 ml serum free DMEM-F12 (5 groups), or (b) 0.5 ml serum free DMEM-F12 (vehicle; 1 group). Blood glucose levels (OneTouch Ultra 2 glucometer) and weights were assessed at baseline and then twice per week for up to 12 weeks.

Canine cell clusters: Two groups of NOD/SCID mice were administered i.p. either (a) 2×10⁵ freshly formed dog cell clusters (N=6) or (b) 0.5 ml serum free DMEM-F12 (vehicle; N=3). Blood glucose levels (OneTouch Ultra 2 glucometer) and weights were assessed at baseline and then twice per week for 10 weeks.

Results

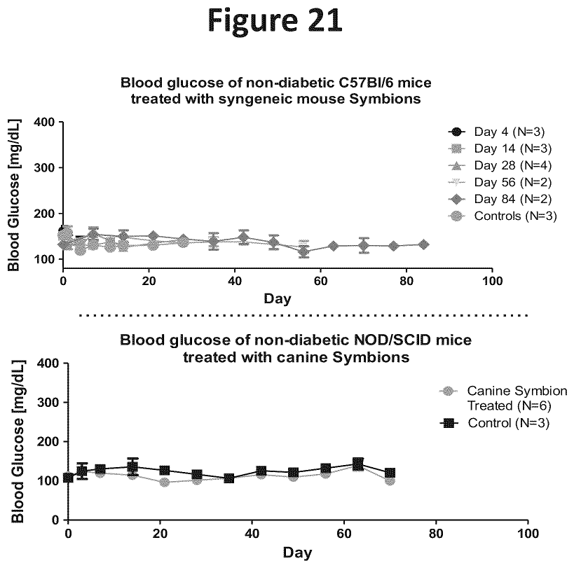

Cell clusters do not cause hypoglycemia in non-diabetic mice. As shown in Example 8 and FIG. 18B, i.p. administered mouse or dog cell clusters engraft in the omentum. As can be seen in FIG. 21, upper panel, blood glucose levels of C57Bl/6 mice that were treated with mouse cell clusters remain normal and comparable to those of vehicle-treated mice. Similar results were obtained for NOD/SCID mice treated with canine cell clusters (FIG. 21, lower panel).

Conclusion: These data demonstrate that engrafted cell clusters formed from either mouse or canine cells release insulin physiologically and not constitutively.

### Example 12: Allogeneic MSCs and Cultured Islet Cells Contained in the Cell Clusters do not Elicit an Antibody Response in Recipients

Rationale: The preceding examples indicate the cell clusters described herein may be used allogeneically to reestablish normoglycemia in diabetic animals without rejection. The following study was undertaken to further test whether animals treated allogeneically with cell clusters produce antibodies to either of the cell types that make up the cell clusters.

Methods

Mouse cell clusters were generated from co-culture in low adherence vessels of P2 Islet cells derived from wild-type C57Bl/6 mice and P5 MSCs derived from C57Bl/6 mice.

Antibody Response Test: Test sera were incubated with either: (a) 1×10⁵ gfp+C57Bl/6 MSCs, or (b) 1×10⁵ cultured C57Bl/6 pancreatic islet cells for 30 minutes. Positive control sera were incubated with 1×10⁵ canine ASCs. After incubation with serum, the cells were centrifuged, resuspended in FACS buffer and incubated with Phycoerythrin (PE) labeled anti-mouse IgG antibody (Pharmingen, San Diego, CA). The cells were incubated an additional 20 minutes in the dark at room temperature. One ml 1×PBS (Roche, Indianapolis, IN)+1% BSA (Sigma, St. Louis, MO) was then added. The cells were vortexed, then centrifuged, resuspended in fixation buffer (1% Formaldehyde), and analyzed by FACS (BD FACScan Analyzer, San Jose, CA; 10,000 cells counted).

Results

Sera were obtained from:
(i) NOD mice that had been treated i.p. with 2×10⁵ cell cluster/kg bw 12 weeks post cell cluster treatment (see Example 8),
(ii) NOD mice that had been treated i.p. with vehicle 12 weeks post vehicle treatment (see Example 8), and
(iii) NOD mice that had not been infused (naïve mice).

Mouse MSCs from cell clusters and mouse pancreatic islet cells from the cell clusters were incubated with the collected sera, and then with Phycoerythrin (PE) labeled anti-mouse IgG antibody. The serum-exposed cells were then analyzed by FACS as described above in Methods to determine whether any IgG antibodies to administered MSCs or pancreatic islet cells were present in the sera of treated mice.

As xenogeneic administration of ASCs is known to elicit an immune response, canine ASCs that had been exposed to sera from NOD mice 14 days post canine ASC administration were incubated with PE labeled anti-mouse IgG antibody, analyzed by FACS, and used as positive controls.

If cell cluster-treated mice had developed an allo-immune response to the MSCs or the pancreatic islet cells in the cell clusters, then the PE-labeled α-mouse IgG antibody would bind to the serum exposed cells, and the cells would appear shifted (PE positive) on FACS analysis. A shift of >7% of the cells (% of PE positive cells) on FACS was considered a positive allo-antibody response.

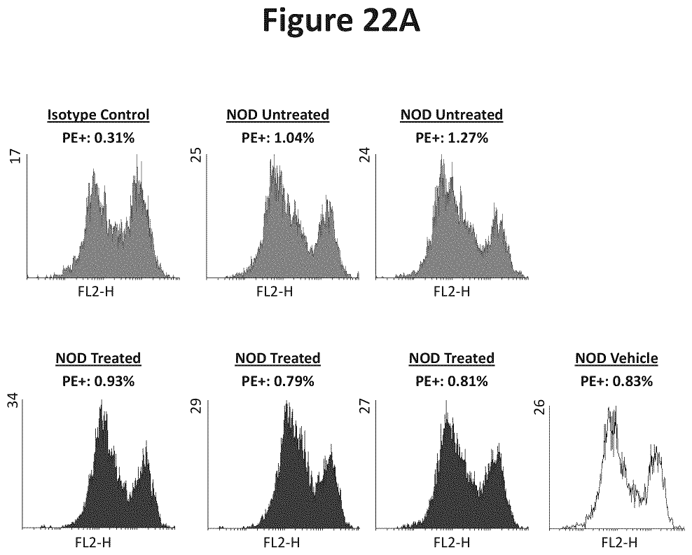

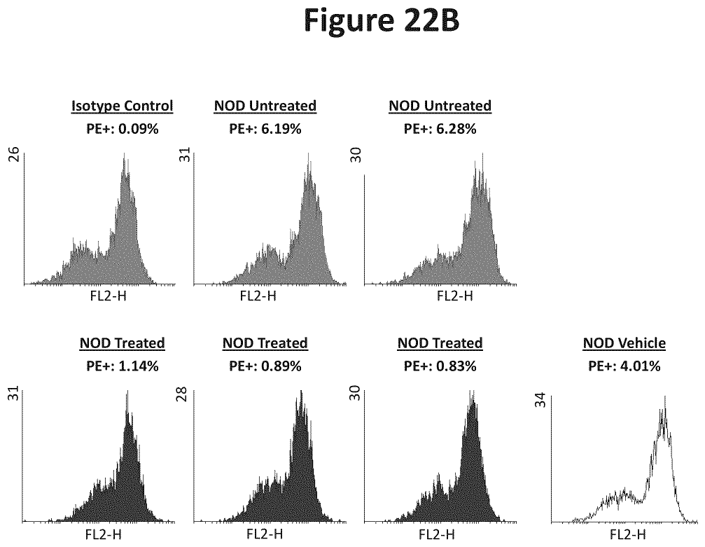

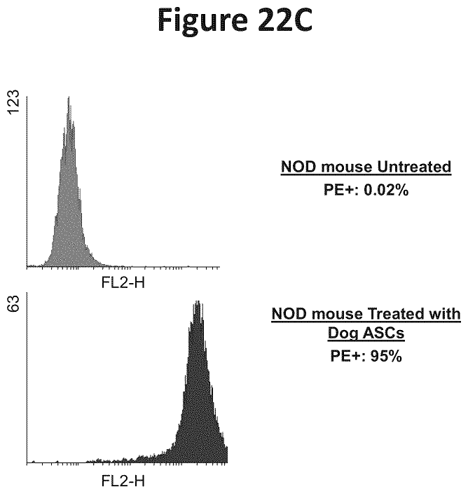

As shown in FIGS. 22A-22C, sera from allogeneic cell cluster-treated mice contained no IgG antibodies to the allogeneic, mouse MSCs (FIG. 22A) or pancreatic islet cells (FIG. 22B). In addition, these data suggest that sera from vehicle-treated NOD mice contained no preformed IgG antibodies against MSCs or dedifferentiated pancreatic islet cells. As expected, sera from mice treated with canine ASCs (positive control) did contain high levels of IgG antibodies to the canine ASCs as evidenced by a shift in 95% of the cells (FIG. 22C).

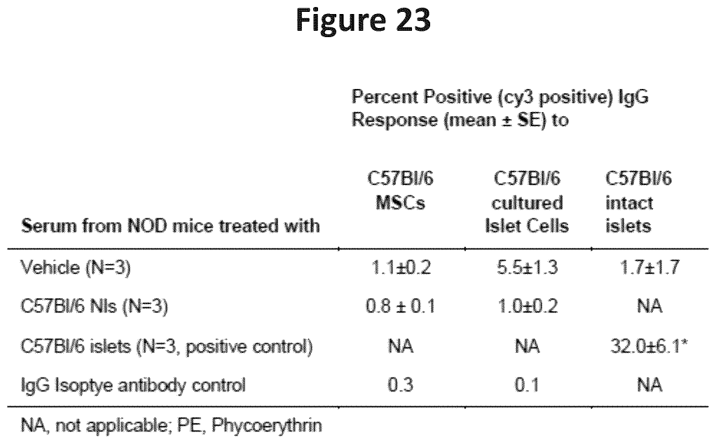

NOD mice do not mount an allo-immune IgG Response to the MSCs and Islet Cells of NIs. To examine whether pancreatic islet cells and MSCs contained in the NIs are protected from a humoral immune attack, we assessed whether sera from normoglycemic, NI-treated NOD mice contained IgG antibodies directed against either the MSCs or cultured ICs that were used to generate the administered NIs. Sera from NI-treated, normoglycemic NOD mice contained neither IgG antibodies directed at MSCs nor at cultured ICs, while the i.p. administration of identical numbers of allogeneic (C57Bl/6), freshly isolated islets used as a positive control, elicited a robust antibody response (FIG. 23). The lack of an IgG antibody response to the cells that are used to form the allogeneic NIs, along with the achievement of long term euglycemia, further indicates that the NIs, as described herein, also provide humoral, allo-immune protection to their islet cell and MSC components.

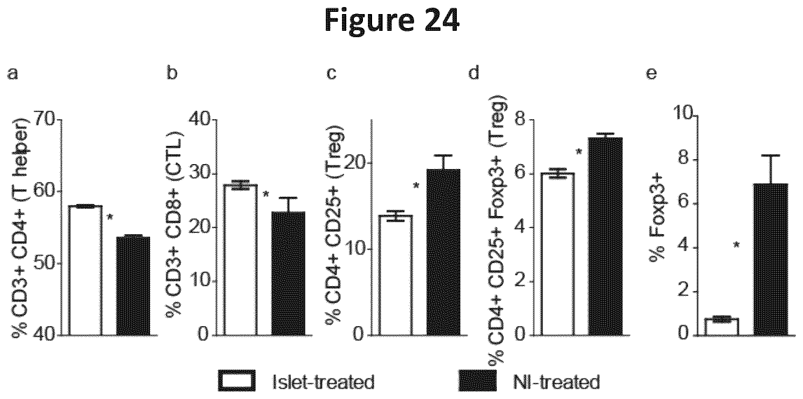

Inhibition of Autoimmune Response. Critical to effectively treating autoimmune T1DM with insulin producing cells is the autoimmune isolation of those cells, and the results presented in FIG. 9 imply that the ICs within the NIs are protected from the autoimmune attack of the treated NOD mice. Autoimmune destruction of beta cells in NOD mice is mediated, as in human T1DM, by autoreactive CD4+ Th1 cells, and is characterized by insulitis involving islet infiltration by macrophages, CD4+ and CD8+ T cells. It has previously been shown that allo-ASC administration either alone or with islets alleviates or prevents hyperglycemia in diabetic animals and humans in part by promoting expansion of regulatory T cells and suppressing expansion of immune cells through here confirmed Tgfb1 expression (FIGS. 3, 8A and 8B) and IDO upregulation in dogs (FIG. 4C). To explore the possibility that the M/ASC component of the NIs protects the pancreatic islet cells from the NOD mouse's autoimmune attack through similar mechanisms, we examined here a select set of known MSC immunomodulatory mechanisms as follows. We treated another group of diabetic NOD mice i.p. with allogeneic C57Bl/6 islets (N=3) or with allogeneic NIs (N=3). After 14 days, such mice were euthanized, and their blood, pancreata, kidneys, lungs, spleens and omenta were harvested. Pancreata were examined histologically and demonstrated to show insulitis as expected. Spleens were harvested and tested by FACS for the percentages of CD3, CD4, CD8, FOXP3, CD25 positive cells. Harvested omenta were examined by IHC for the presence of Foxp3+ cells. The percent of CD3/CD4 and CD3/CD8 double positive cells (helper and cytotoxic T Lymphocytes) were significantly lower in spleen cells of NI-treated vs. Islet treated NOD mice, while the percent of CD4/CD25 double positive and CD4/CD25/Foxp3 triple positive Tregs were significantly increased in the spleens of M-treated vs. Islet treated NOD mice (FACS analysis, FIG. 24, Panels a-d). Similarly, IHC analysis of omenta of M treated mice showed a significant increase in the percent of Foxp3 positive cells vs. those of vehicle treated mice (FIG. 24, Panel e). While the number of animals tested is small, these results are in agreement with others' findings and with our hypothesis that NIs, and specifically their M/ASC component, promotes euglycemia in T1-diabetic mice through modulation of the diabetogenic auto-immune response. Omenta from the above mice were also stained for Ki67 to examine whether there was significant cell division associated with M grafts. None was found.

Conclusion: The above data indicate that administration of cell clusters does not elicit an antibody response to either cell type that composes the cell cluster, further supporting the hypothesis that the cell clusters provide immune isolation and eliminate the need for anti-rejection drugs and encapsulation devices.

Our extensive in vitro and in vivo data to date and presented above demonstrate that the treatment of experimental T1DM in mice with syngeneic and allogeneic cell clusters, and cell clusters from multiple species are able to effectively re-establish euglycemia, i.e., treat T1DM, and this during long-term follow-up. No Adverse Events, such as oncogenic transformation or ectopic maldifferentiation of cell clusters were observed. This novel therapy can be used as treatment of insulin-dependent diabetes both in companion animals (dogs, cats) and humans with type 1 diabetes mellitus.

### Example 13: Human Derived Cell Clusters Used for the Treatment of STZ Diabetic NOD/SCID Mice

Cell clusters containing human cells are generated as described in the above examples using ASC and/or MSCs from human subjects identified as healthy and not suffering from insulin-dependent Diabetes Mellitus and pancreatic islet cells from an allogeneic source.

The purpose of this study was to determine whether human cell derived cell clusters (hNIs) could reduce or eliminate the need for insulin in diabetic NOD/SCID mice as we previously found for dog cell derived cell clusters (cNIs).[3] Specifically, we set out (a) to determine whether passaged human islet cells (hICs) are characteristically comparable to canine islet cells (cICs), both in gene expression and response to cytokines, etc.; and (b) to determine if human hNIs can durably reduce or eliminate the need for insulin as cNIs and mNIs have been shown to do.[3]

Research grade human islets from 8 non-diabetic human donors (see Table A for demographics and Islet Viability) were purchased in lots of ˜5,000 Islet Equivalents from Prodo Labs (Aliso Viejo, CA). Islet cells derived from this inhomogeneous group of islet donors were expanded by culturing whole islets in tissue culture flasks, using RPMI 1640 medium (Gibco, Thermo Fisher Scientific, Waltham, MA)+10% human Platelet Lysate (hPL; Cell Therapy and Regenerative Medicine, University of Utah, Salt Lake City)+1× L-Glutamine-Penicillin-Streptomycin solution (GPS; Sigma G1146) until ˜90% confluent. For passaging, cells were released with 2× Trypsin (Sigma, St. Louis, MO), pelleted by centrifugation at 600×g for 5 min., washed with DMEM 5 mM glucose (Gibco)+10% hPL+GPS (complete medium), and reseeded at a density of 2×10e5 cells into T75 flasks in complete medium. hICs were characterized by rtPCR for expression of IC specific genes. hIC and cIC doubling times and population doublings (PDLs) were calculated by standard methods.

TABLE A
Demographics Characteristics and Islet Viability of human Islet Donors

| | Age | | | | HbA1c | | Viability of |
|---|---|---|---|---|---|---|---|
| Donor # | [yrs] | Gender | Race | Cause of Death | [%] | BMI | Islets |
| 1 | 27 | Male | Native | head trauma | 5.5 | 25.8 | 80% |
| | | | American | | | | |
| 2 | 28 | Male | Caucasian | head trauma | 4.2 | 34.7 | 80% |
| 3 | 40 | Male | Hispanic | head trauma | 5.3 | 25.3 | 70% |
| 4 | 48 | Female | Asian | stroke | 5.3 | 21.5 | 90% |
| 5 | 29 | Male | Hispanic | head trauma | 5.5 | 22.8 | 95% |
| 6 | 61 | Female | Hispanic | head trauma | 5.2 | 28.9 | 90% |
| 7(15) | 21 | Male | Caucasian | head trauma | 5.2% | 22.8 | 95% |
| 8(19) | 41 | Male | Caucasian | head trauma | 5.3% | 28.0 | 90% |

MSC culture: Human, bone marrow derived MSCs were purchased pre-characterized (tri-lineage differentiation, HLA antigens and surface CD markers) from Lonza (Walkersville, MD) and cultured in complete medium as previously described [2,20,21], and used at Passage 3 for formation of NIs.

Dog islets and cell lines: Utilized dog islets and Adipose Stem Cells (ASCs) from inguinal fat were identical to those used in our ongoing pilot study (INAD 012-776) [3] and all dog cell lines were cultured as previously described [3]. All dogs were non-diabetic mongrels, but some had pacemaker-induced congestive heart failure (see Table B for details). Both islets and adipose tissue were obtained through an NIH Organ Sharing Agreement at the University of Utah.

TABLE B
Demographics Characteristics of canine Pancreas/Islet Donors and Number of
Islets isolated per Pancreas (cause of death in all donors was euthanasia)

| Donor | Age | | | Pancreas | # islets | weight | Viability |
|---|---|---|---|---|---|---|---|
| # | [yrs] | Gender | Health | weight [g] | isolated | [kg] | of Islets |
| 1 | 1.3 | Female | CHF* | 54 | ND** | 25 | 80% |
| 2 | 1.3 | Male | healthy | 30 | 48,000 | 27 | 95% |
| 3 | 1.3 | Male | CHF | 51.7 | 30,000 | 28.8 | 60% |
| 4 | 1.4 | Male | healthy | 45 | 60,000 | 24 | 90% |
| 5 | 1.2 | Male | healthy | 54 | 60,000 | 27 | 65% |
| 6 | 2.1 | Male | CHF | 52 | 60,000 | 25.6 | 75% |

*CHF = Pacemaker-induced Congestive Heart Failure.
**ND = not determined

Islet and cell viability were assessed using Fluorescein diacetate (FDA, Sigma F7378) and Propidium Iodide (PI, Life Technologies P3566) staining, following instructions of the respective manufacturers. Islet viability in percent was quantified in 10 different, homogeneously distributed fields of ˜400 human and canine islets. This method does not detect potential apoptotic cell loss.

Human MSCs and canine ASCs and respective ICs were co-cultured in complete medium at a 1:1 ratio in ultra-low adhesion surface culture dishes (Corning, Kennebunk, ME), resulting in highly efficient cell cluster (hNI) and (cNI) formation overnight, as previously reported [2,3].

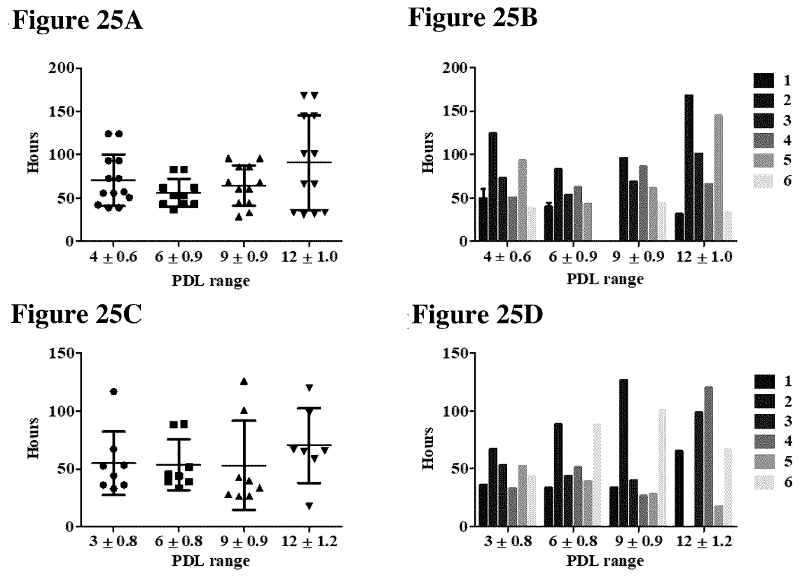

Population Doubling Times: We previously observed that PDLs of cultured mouse and dog islet cells between different donors vary significantly. However, this inter-donor variability did not affect subsequent function of either mouse or dog cell clusters in vivo. In the present study, doubling times of hICs and cICSs over 4-12 population doublings (PDLs) are shown in FIGS. 25A and 24B for hICs and 25C and 25D for cICs. Doubling times ranged from 29 to 170 hrs, with a mean of ˜70 hrs. Such variability of doubling times parallels those we previously observed in both mouse and dog ICs.

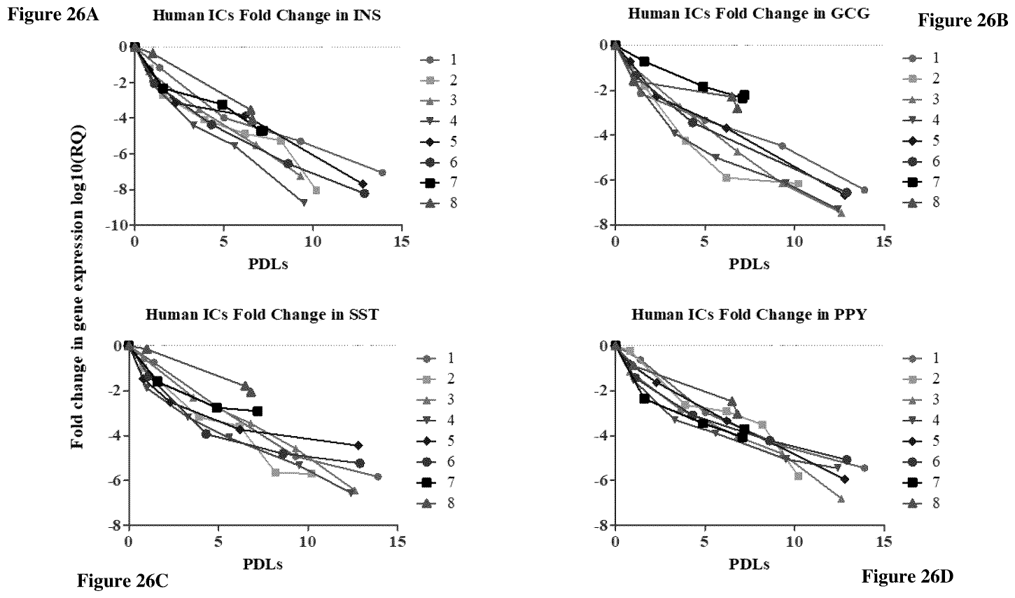

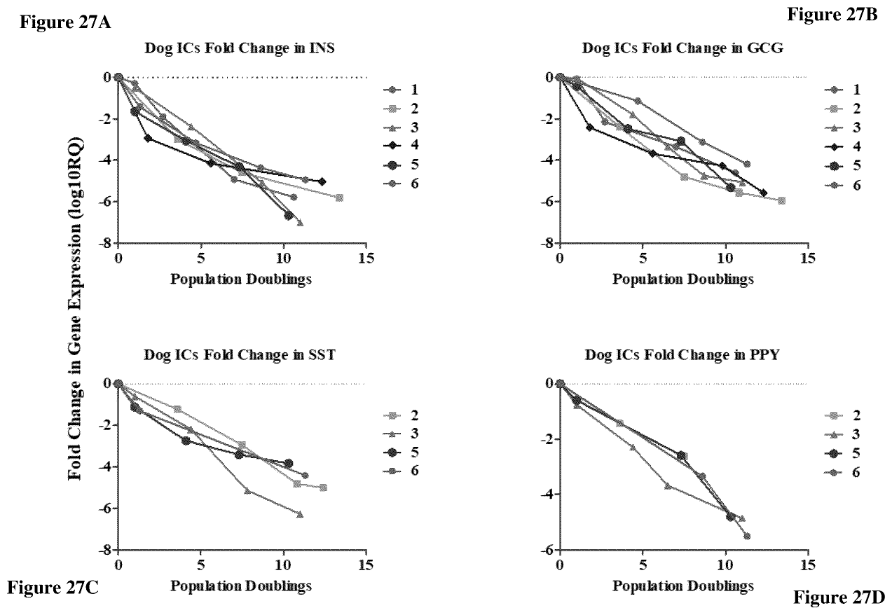

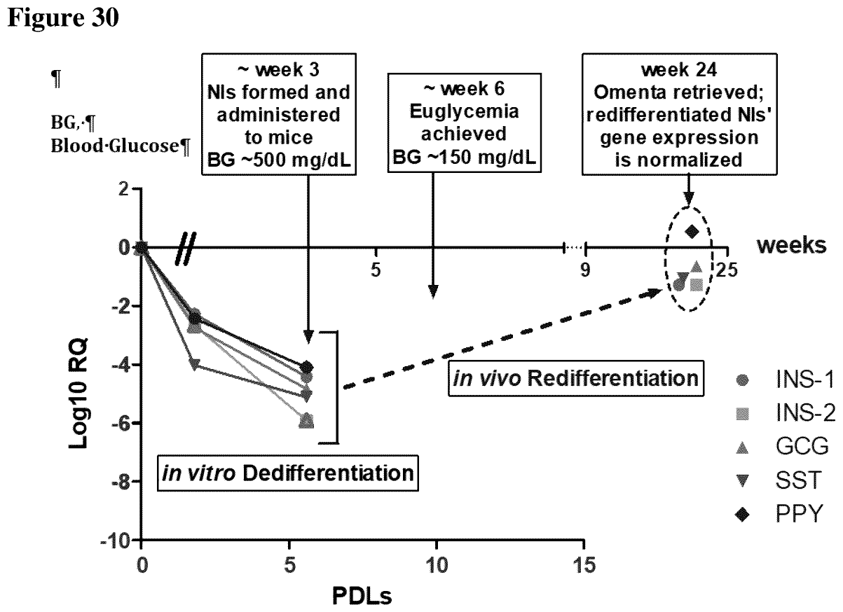

GSIS and Gene Expression Profiles of Insulin and other Islet-specific Hormones as a Function of Population Doublings (PDLs): For the ongoing cell cluster treatment of diabetic dogs, insulin and other islet hormone gene expression levels are assessed in cultured cICs prior to cell cluster formation, and insulin gene expression is used as a measure of potency and serves as release criterion for cICs and canine cell cluster. In order to determine whether hICs (n=6) and cICs (n=6) that are being culture expanded per identical protocol are functionally comparable, we systematically assessed in the cell lines from both species the gene expression levels of insulin (INS), glucagon (GCG), somatostatin (SST), and pancreatic polypeptide (PPY) by rtPCR (see Table C for rtPCR primers), and plotted these as a function of PDLs (see Table D for details). As shown in FIGS. 26 and 27, for both human ICs (FIGS. 26A-26D) and dog ICs (FIGS. 27A-27D), the levels of INS, GCG, SST, and PPY, expressed as a function of PDLs remained consistent and detectible through at least 10 PDLs, and these were strikingly donor independent. The progressive decreases in insulin and other islet hormone gene expression levels in culture expanded hICs and cICs were linear (R-squared>0.91), and these results were comparable to those observed in mouse ICs (see FIG. 30). With the exception of dog SST, changes between slopes among the donors were not significantly different for any of the assessed genes. Similarly, expression levels and extrapolated line elevations were not significantly different from each other between donors. In further support of the similarity of cultured IC behavior among species, dog ICs, like mouse ICs, have been shown to readily redifferentiate to produce physiologic levels of insulin and other islet hormones in vivo. Administration of dog cell clusters to spontaneously diabetic pet dogs reduces the need for insulin long term, demonstrating that the phenomenon of IC redifferentiation in vivo occurs in dogs as well. Finally, when ICs were incorporated into cell clusters and administered i.p. to diabetic mice, mouse (FIG. 30) and dog ICs were shown to redifferentiate in vivo and to produce physiologic levels of insulin. The parallel results observed in FIGS. 26A and 27A for culture-expanded human vs. dog ICs support our hypothesis that the cell cluster technology, already demonstrated to eliminate or reduce the need for insulin in diabetic mice and dogs, respectively, possesses substantial translational promise for its clinical testing and subsequent treatment of human T1DM.

TABLE C
rtPCR primers used to test Human and Dog Target Gene Expression

| | Applied Biosystems |
|---|---|
| Target genes | catalog # |
| ACTB (human) | Hs01060665_g1 |
| B2M (human) | Hs00984230_m1 |
| INS (human) | Hs02741908_m1 |
| GCG (human) | Hs01031536_ml |
| SST (human) | Hs00356144_m1 |
| PPY (human) | Hs00358111_g1 |
| ACTB (dog) | Cf03023880_g1 |
| B2M (dog) | Cf02659077_m1 |
| INS (dog) | Cf02647520_ml |
| GCG (dog) | Cf02624195_m1 |
| SST (dog) | Cf02625293_m1 |
| PPY (dog) | Cf02653446_g1 |

TABLE D
R-squared values for observed decreases in Human and Dog endocrine Gene
Expression Levels as function of PDLs, normalized to Parent Cells

| | Human | | | | Dog | | | |
|---|---|---|---|---|---|---|---|---|
| Donor | INS | GCG | SST | PPY | INS | GCG | SST | PPY |
| 1 | 0.9593 | 0.9371 | 0.9688 | 0.957 | 0.9637 | 0.938 | ND | ND |
| 2 | 0.931 | 0.8742 | 0.9591 | 0.9435 | 0.8998 | 0.9382 | 0.9896 | 0.9942 |
| 3 | 0.9862 | 0.9886 | 0.9936 | 0.9768 | 0.9941 | 0.9701 | 0.984 | 0.9737 |
| 4 | 0.9678 | 0.9138 | 0.9333 | 0.8781 | 0.9143 | 0.8868 | ND | ND |
| 5 | 0.9436 | 0.9742 | 0.796 | 0.9886 | 0.9685 | 0.9707 | 0.897 | 0.9659 |
| 6 | 0.9505 | 0.9566 | 0.8357 | 0.9123 | 0.9428 | 0.9769 | 0.9667 | 0.9705 |
| Ave | 0.9564 | 0.94075 | 0.914417 | 0.942717 | 0.9472 | 0.946783 | 0.959325 | 0.976075 |

ND, not determined; SST and PPY gene expression levels on 2 dog cell lines were not determined.

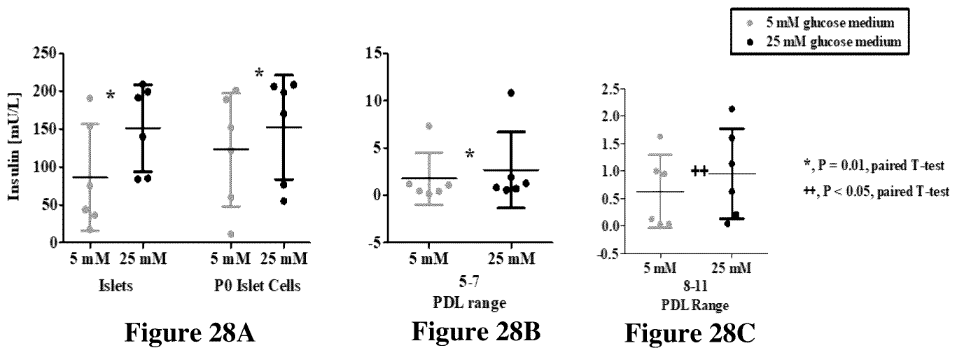

We previously reported that culture expanded mouse and dog ICs and cell clusters secrete insulin in response to glucose stimulation, albeit at significantly reduced levels compared to freshly isolated, whole islets [2,3]. Furthermore, dog cell clusters implanted into streptozotocin-diabetic NOD-SCID mice durably induced euglycemia. When retrieved 9 weeks later, these canine cell clusters secreted 15-fold higher concentrations of canine insulin in response to glucose stimulation than did freshly formed dog cell clusters, clearly demonstrating that they had re-differentiated in vivo. To assess whether culture expanded human ICs also secrete insulin in response to glucose, culture expanded hICs from the donors in Table A were tested per GSIS at different passages and their insulin secretion was compared with that of their parent islets (FIGS. 28A-28C). As with dog and mouse ICs, culture expanded hICs show glucose stimulated insulin secretion through 8-11 PDLs, but again at reduced levels compared to freshly isolated, native islets (FIGS. 28A-28C).

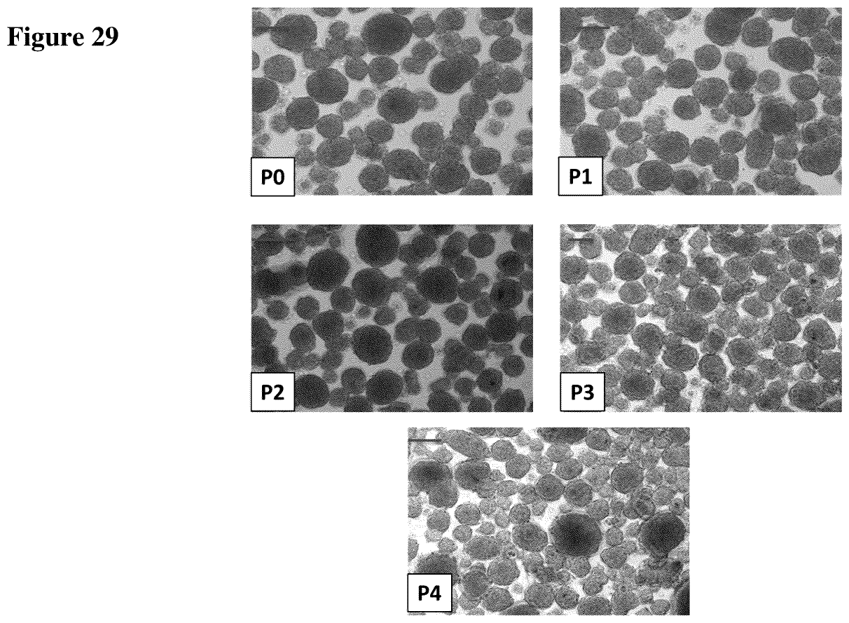

Cell cluster formation: Islet cells from the human donors listed in Table A were tested at passages P0-P4 for their ability to form cell clusters when co-cultured with human MSCs (P3) as described in Methods. Representative images of such freshly formed cell clusters are shown in FIG. 29. Islet cells from all donors and from these passages readily formed phenotypically comparable cell clusters, indicating that human donor variations are unlikely to be a significant factor for this aspect of the cell cluster technology.

Treatment of Mice with Human Cell Clusters

Passaged hICs in human cell clusters that were used to treat diabetic NOD/SCID mice. For in vivo testing, NOD/SCID mice were made diabetic with STZ, treated with insulin pellets (Linbits), and once stabilized, treated i.p. either with ˜2×10e5 NI/kg bw or vehicle, then followed for 7 weeks. To help assess the role of donor variability, two sets of NIs using hICs from different donors (donors 7 and 8 of Table A) and hMSCs were formed. At the beginning of the 7^{th} week, all animals and a group of healthy controls underwent intraperitoneal Glucose Tolerance Tests (IP GTTs).

As was previously found with cNIs [2,3], hNIs dosed at ˜2×10e5 NIs per kg bw durably restore euglycemia as demonstrated by normal IP GTTs and elimination of the need for insulin in diabetic NOD/SCID mice.

Results:

Two sets of hNIs were formed, each set incorporating P3 clinical grade hMSCs and P1 islet cells from donors 7 and 8 of the research grade donors listed in Table A.

hMSCs were shown to undergo trilineage differentiation and to express MSC specific epitopes and genes. [20,21]

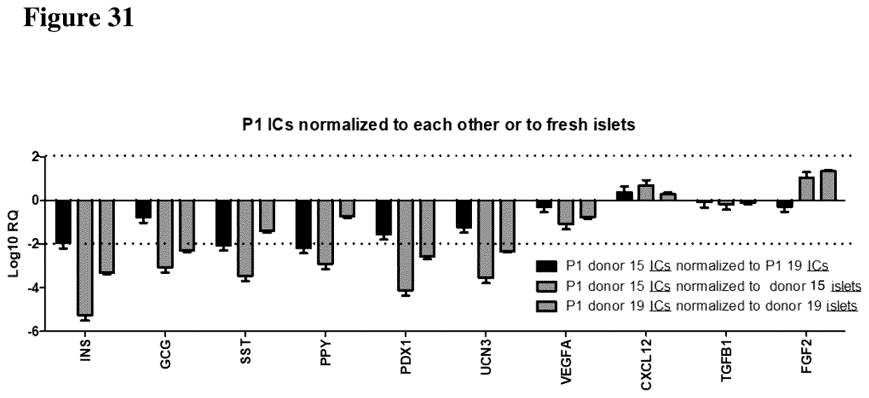

Passage 1 (P1) islet cells and NIs composed of islet cells from donor 7 and donor 8 and hMSCs were characterized by rtPCR for expression of islet-specific genes of interest and compared to each other as well as to fresh islets (FIGS. 26A-26D). As expected from previous experiments conducted using mouse and dog cells, [2,3] and as shown in FIG. 31, while passage 1 (P1) islet cells from both donors expressed INS, GCG, SST, PPY, PDX1, and UCN3 mRNA, P1 islet cells from both donors expressed significantly reduced levels of all assayed islet cell genes as compared to freshly isolated islets (green and orange bars). Likely as a reflection of donor variability, donor 7 P1 islet cells expressed significantly lower levels of mRNA than did donor 8's P1 islet cells (black bars of FIG. 31).

In FIG. 31 shown are the gene expression levels of the P1 ICs cultured from the islets of each of donors 7 and 8 listed in Table A. The black bars show them normalized to each other. The green and orange bars show them normalized to the freshly isolated islets from which the P1 cells were cultured. Log 10 RQ of ±2 is considered significantly different. P1 ICs from donor 7 express significantly lower levels of IC associated genes than donor 8's P1 ICs.

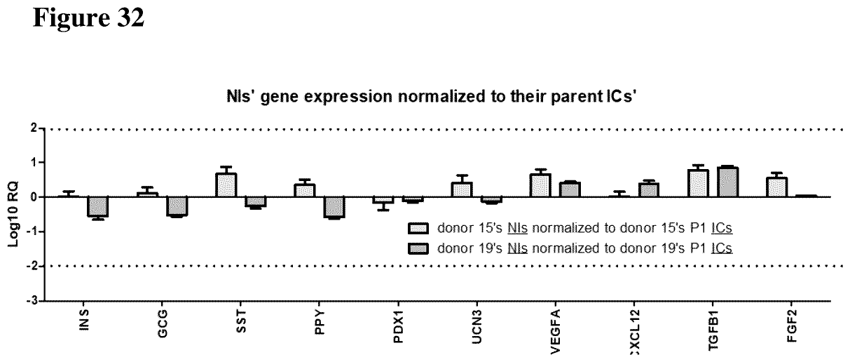

Incorporation into cell clusters of P1 ICs from either donor did not significantly affect expression of any assayed gene (see FIG. 32).

As shown in FIG. 32, gene expression levels of cell clusters made from donor 7's and donor 8's P1 ICs normalized in each case to the donors' P1 ICs' expression levels prior to incorporation. Incorporation of ICs into cell clusters does not significantly alter the gene expression levels from what they were prior to the cells' incorporation (FIG. 31).

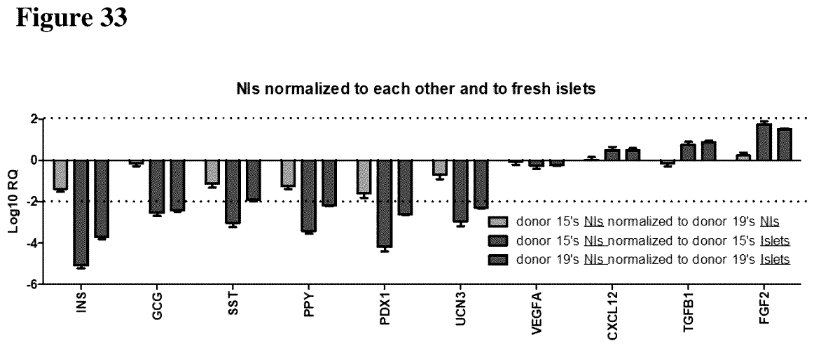

Once ICs were incorporated into cell clusters, Islet-specific gene expression levels from donor 7 NIs were still reduced compared to those of donor 8 NIs, but not significantly (FIG. 33; grey bars).

FIG. 33 shows that the significant differences seen in FIG. 31 in islet associated gene expression between the two donors is eliminated upon incorporation of the ICs into cell clusters (grey bars). The cell clusters used for treatment are therefore roughly equivalent.

Also shown in FIG. 33 are the gene expression levels of cell clusters compared to those of the donors' original islets (green and red bars). As expected, islet associated gene expression levels are significantly reduced in the NIs but are still detectible.

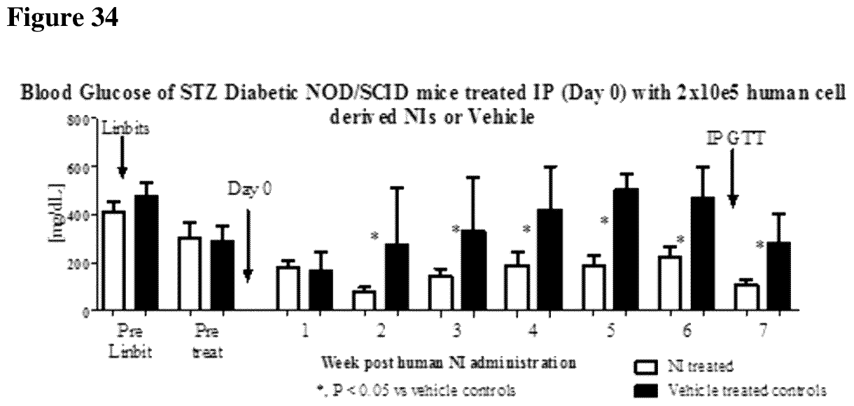

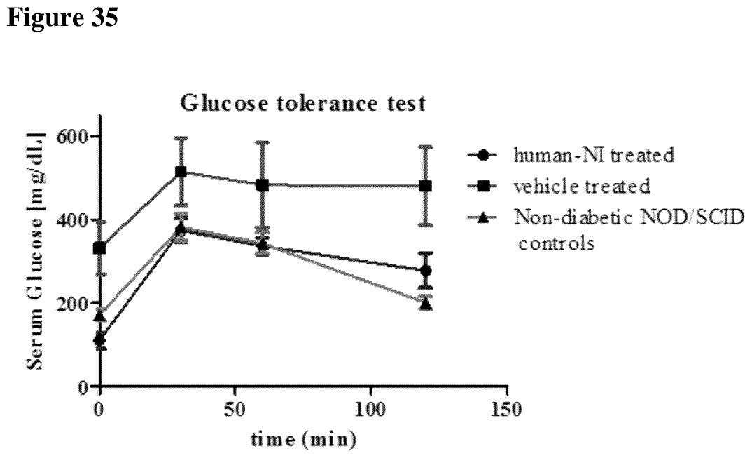

Use of hNIs in NOD/SCID mice for treatment of STZ induced Diabetes mellitus: 12 female, 13 week old NOD/SCID mice were made diabetic with one or two doses of STZ, 200 mg i.p. as described in Methods. Blood glucose levels were monitored 2× per week, and mice were considered diabetic when such levels were >300 mg/dL for 3 consecutive days, at which point, mice were treated with sub-cutaneous insulin (Linbit) pellets. Once blood glucose levels were controlled to <200 mg/dL, mice were divided into two groups of six mice each and treated i.p. either with (i) ˜2×10e5 NI/kg bw or (ii) vehicle (500 uL αMEM). For the 6 hNI treated mice, 3 were treated with NIs that incorporated donor 7's ICs, while the other 3 used donor 8's ICs. Treatment with cells from either donor offered comparable responses. As shown in FIG. 34, treatment with hNIs resulted in durable euglycemia and elimination of the need for insulin while treatment with vehicle did not. IP GTTs were essentially normal in cell cluster treated, but not vehicle treated animals (FIG. 35). Importantly, the species of insulin produced in cell cluster treated mice during the IP GTT was exclusively of human origin.

### Example 14: Intraperitoneal Administration of Human “Neo-Islets”, 3-D Organoids of Mesenchymal Stromal and Pancreatic Islet Cells, Normalizes Blood Glucose Levels in Streptozotocin-Diabetic NOD/SCID Mice

Study Design

The current, preclinical study was undertaken in anticipation of a Phase 1 Clinical Trial with two objectives: to determine (a) whether human NIs (hNIs) can also restore euglycemia, and (b) whether redosing of suboptimally controlled diabetic animals could fully restore euglycemia in streptozotocin (STC)-diabetic Non-Obese Diabetic/Severe Combined Immunodeficiency mice (NOD/SCID, Harlan), as has been previously shown for mouse cell-derived NIs (mNIs), and dog cell-derived NIs (cNIs)[3]. Since these NIs are composed of human cells, and since human MSCs do not maintain immune evasive abilities in a xenogeneic setting (unpublished results), the NOD/SCID model was used. It does reproduce, in part, the clinical situation in which recipients of allogeneic biotherapies must permanently take potent anti-rejection drugs that similarly create a life-long immune-compromised status. Passaged hICs and hNIs that were to be used to treat diabetic NOD/SCID mice were characterized for gene expression profiles by rtPCR. For in vivo testing, NOD/SCID mice were made diabetic with STZ, then randomized based on blood glucose levels into groups of 6 each. Following randomization, the mice were administered insulin pellets (Linbits, Linshin Canada) to control blood glucose levels and prevent glucotoxicity and enhance in vivo redifferentiation of the hICs within the graft. Once blood glucose levels were stabilized near normal, animals were treated i.p. either with ˜2×10e5 human cell-derived NIs/kg bw (n=6) or vehicle (n=6), then followed for 8 weeks. Once placed in a group, and until endpoint or euthanasia for humane reasons, data from all animals were included in subsequent analyses. Once blood glucose levels were determined to be no longer significantly improved compared to controls without administration of exogenous insulin, mice in each group were again treated with either 2×10e5 NIs/kg bw or vehicle, and followed for an additional 6 weeks.

Animal Model

Animal studies were conducted in adherence to the NIH Guide for the Care and Use of Laboratory Animals, and were supervised and approved by an institutional veterinarian and member of the IACUC.

Care. NOD/SCID mice were maintained in a sterile environment, and provided with sterile bedding, food and water. They were kept in a temperature and humidity controlled environment on a 12 hr light dark cycle and given free access to food and water Animal health and behavior were visually observed at least once a day during the work week, and by blood glucose and weight checks at least 2 times a week by staff, each of whom had completed CITI training in the care of rodents, and had at least 2 years' experience with mice and the procedures herein described.

Induction of diabetes and treatment. 12 female, 13 week old NOD/SCID mice were made diabetic with one to two i.p. doses of Streptozotocin (STZ; Sigma), 200 mg ip dissolved in citrate buffer (pH 4.5; Sigma), and administered under light anesthesia as described below. Tail vein blood glucose levels were monitored 2× per week, and mice were considered diabetic when such levels were >300 mg/dL for 3 consecutive days, at which point, mice were lightly anesthetized and treated with sub-cutaneous, slow-release insulin (Linbit) pellets. Once blood glucose levels were controlled to <200 mg/dL, the two groups of six mice each were lightly anesthetized, and treated i.p. either with (i) 2×10e5 human cell derived NIs (hNIs/kg b.wt.; in 500 uL DMEM (5 mM glucose) (Gibco)) or (ii) vehicle (500 uL DMEM (5 mM glucose)).

Anesthesia. Mice were anesthetized with isoflurane (Baxter), 1-5%, using an inhalation rodent anesthesia system (Euthanex). Rectal temperatures were maintained at 37° C. using a heated surgical waterbed (Euthanex).

Blood glucose and weight monitoring. Blood glucose concentrations were assessed twice per week via sterile tail vein sampling, using a 27-30 gauge needle to obtain a drop of blood, and a OneTouch Ultra 2 glucometer (level of detection, 20-600 mg glucose/dL; LifeScan). Post blood sampling, mice were observed until bleeding stopped and for a short time after for signs of tail bruising or pain (hunched appearance, head pressing, etc.). As anesthesia results in a rise of blood glucose, anesthesia was not used for blood glucose monitoring. Care was taken to minimize the pain and distress caused to mice required by handling and blood sampling for glucose monitoring, and analgesics were available as described in pain management below for any animal showing signs of pain from tail vein sampling. Animal weight was assessed twice weekly in conjunction with blood glucose monitoring.

Intraperitoneal Glucose Tolerance Tests (i.p. GTTs) and assay of human insulin. At indicated time-points post 1^{st} and 2^{nd} doses of hNIs, vehicle-treated and hNI-treated NOD/SCID mice, and an additional group of age-matched, non-diabetic NOD/SCID control mice (n=6) were fasted hrs, whereupon baseline blood glucose levels were measured Animals were anesthetized and 2 g glucose/kg b.wt. (dissolved in 0.5 ml serum free medium and filter sterilized; Sigma, St. Louis, MO) were administered via i.p. injection. Tail vein blood glucose levels were determined at 30 min, 60 min and 120 min post glucose administration. Human insulin levels in the sera of hNI and vehicle treated groups of mice were assayed by ELISA, following the manufacturer's instructions (Mercodia, Uppsala, Sweden). Pain management. Buprenorphine 0.05 mg/kg bw IM was available as needed for any animal appearing to suffer from pain following i.p. STZ administration, NI administration, i.p. glucose tolerance testing, or tail vein sampling.

Endpoint criteria. For all mice in this study, the following criteria were used to determine whether they should be removed from the protocol or euthanized to prevent suffering: Animals that exhibited evidence of poor health, including weight loss greater than 20%, excessive wasting (>20% compared to age/sex matched littermates), ungroomed appearance, poor activity level, labored breathing or loss of appetite/water intake, neoplasia, stupor, severe injury due to fighting with cage mates, any signs of abnormal behavior including severe aggressiveness towards handler or cage mates such as to inflict injury, lack of physical or mental alertness, or any animal appearing to be in grave distress Animals beginning to show signs of distress were monitored daily and carefully observed for general appearance, behavior and weight loss. Any animal appearing to be in grave distress or to have weight loss or muscle wasting of 20% or more were immediately euthanized to prevent further suffering.

No animal died before meeting endpoint criteria or study endpoint, but four mice in the vehicle treatment group met the criteria for euthanasia (all four exhibited excessive wasting and lack of appetite, combined with ungroomed appearance), and were euthanized on days 46 (3 mice) and 56 (1 mouse) as detailed in “Euthanasia” below, and as soon as they met those criteria.

Euthanasia. At study endpoint (applied to 8 of the 12 mice in the study), 15 weeks post first treatment with STZ, and where necessary as defined by endpoint criteria (applied to 4 mice in the vehicle treatment group) mice were euthanized using CO2 gas/4-5 L over 2-4 minutes. Death was verified by the assurance of the cessation of respiratory and cardiovascular movements by observation for at least 10 minutes.

Cells

NIs are composed of equal numbers of culture-expanded human MSCs and human Islet Cells, which spontaneously form clusters when co-cultured. Culture and NI formation are detailed below.

Islet cell culture. Research grade human islets from adult, non-diabetic donors were purchased from Prodo Labs. Islet cells were cultured by placing whole islets into tissue culture flasks and culturing them in RPMI 1640 (Life Technologies)+10% human Platelet Lysate (hPL; Cell Therapy and Regenerative Medicine, Salt Lake City, UT)+Gentamycin, Penicillin, Streptomycin (GPS; Sigma) until 90% confluent. For passaging, cells were trypsinized using 1× Trypsin EDTA (Sigma), pelleted by centrifugation at 600×g for 5 min, washed with DMEM (5 mM glucose)+10% hPL+GPS, and reseeded at a density of 2×10e5 cells into Cell Bind coated T75 flasks (Corning). Cultured Islet Cells (IC) were used at Passage 1.

MSC culture. Human, bone marrow derived MSCs were purchased from Lonza (Walkersville, MD) and cultured as previously described. MSCs were used at P3 for NI formation.

Neo-Islet (NI) formation. MSCs and Islet cells were co-cultured in DMEM (5 mM glucose)+10% hPL at a 1:1 ratio in ultra-low adhesion surface culture dishes (Corning), and NIs formed overnight as previously described.

rtPCR

Prior to in vivo administration, NIs were tested by rtPCR for expression of islet-associated genes INS, GCG, SST, PPY, PDX1, and UCN3. rtPCR was carried out as previously described, using the reagents and primers listed in. In brief, Relative Quantification, (RQ; defined as is standard as 2-MCT where CT is the Cycle Threshold), was calculated through normalization to internal (deltaCT; beta actin and beta 2 microglobulin) and external controls (delta-deltaCT; parent cells), both accomplished using the ABS 7500 Real Time PCR System and software. Results are presented as log 10(RQ)±log 10(RQmin and RQmax) so that up- and down-gene regulation is represented equally. Differences between expression levels greater than log 10(RQ) 2 or log 10(RQ)-2 were considered significant.

Statistical Analysis

Data are expressed as Mean±SEM or Mean±95% confidence interval, as indicated. Primary data were collected using Excel (Microsoft, Redmond, WA), and statistical analyses were carried out using Prism (GraphPad). Two tailed t-tests were used to assess differences between data means. A P value of <0.05 was considered significant. For rtPCR, data are presented as Log 10RQ, and statistical significance is defined as ±2.

Results

Reduced Levels of Islet-Associated Genes are Expressed in hNIs

hNIs were formed, each set incorporating P3 hMSCs and P1 islet cells from non-diabetic, adult human donors. hMSCs were obtained pre-characterized for expression of MSC-specific epitopes and genes, and for their ability to undergo trilineage differentiation (adipo-, osteo- and chondrogenic) (Lonza).

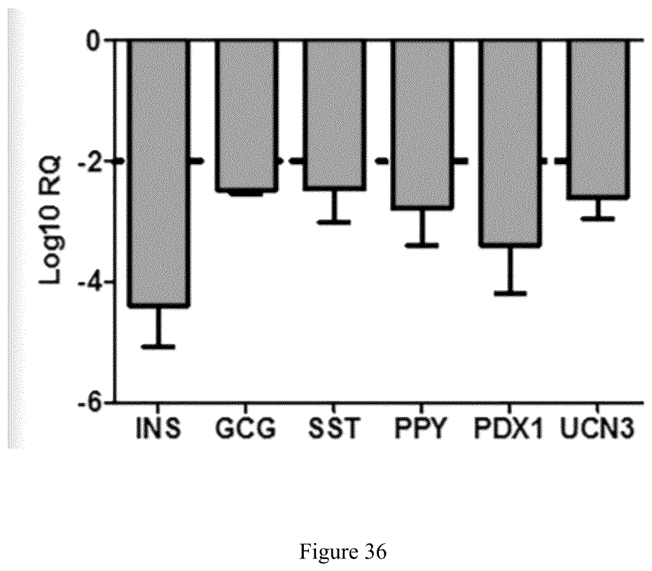

Gene expression analysis was conducted using rtPCR on the freshly formed NIs to determine their expression levels of islet endocrine genes as compared to those of whole islets. As expected from previous experiments using mouse and dog cells, and as shown in FIG. 36, NIs containing P1 islet cells expressed INS, GCG, SST, PPY, PDX1, and UCN3 mRNAs. Also as was previously found for mouse and dog cells, respective expression levels of these islet cell genes were significantly reduced compared to freshly isolated human islets. In other words, islet associated genes (INS, GCG, SST, PPY, PDX1, and UCN3) are expressed in human NIs prior to administration, but at significantly reduced levels compared to those of freshly isolated human islets, comparable to what was previously found for dog and mouse culture-expanded islet cells.

Therapeutic Efficacy of hNIs

A single dose of hNIs improves glycemic control in diabetic mice. In order to assess the therapeutic efficacy of hNIs for the treatment of insulin-dependent DM, Diabetes was established in 12 female NOD/SCID mice, after which they were randomized into 2 groups of 6 mice each, and their blood glucose levels were controlled with slow-release insulin pellets (Linbits). Once blood glucose levels were controlled, mice were treated either with vehicle or hNIs as described in Methods. After this treatment, mice were followed for 8 weeks, at which time, an i.p GTT was conducted as described in Methods, in conjunction with an ELISA assay to detect the presence of human Insulin.

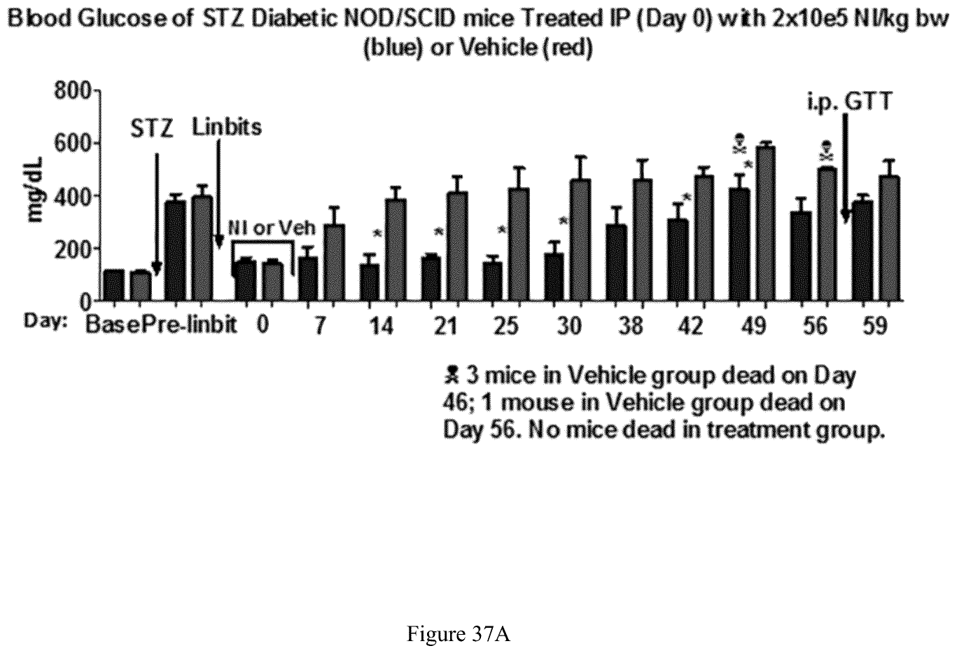

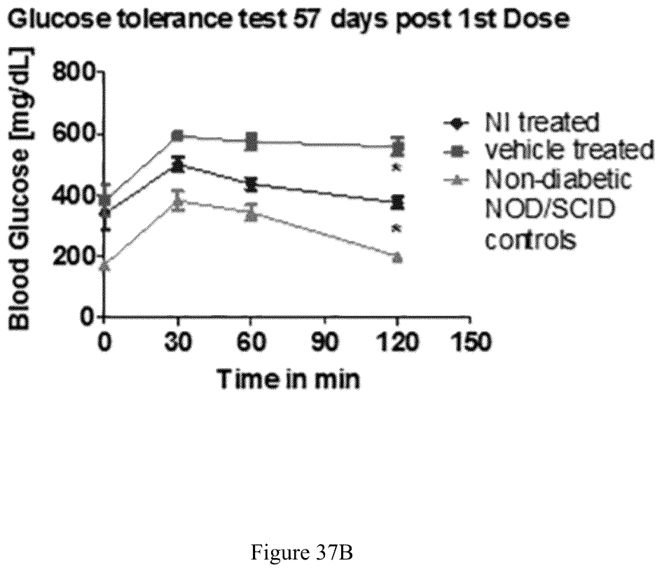

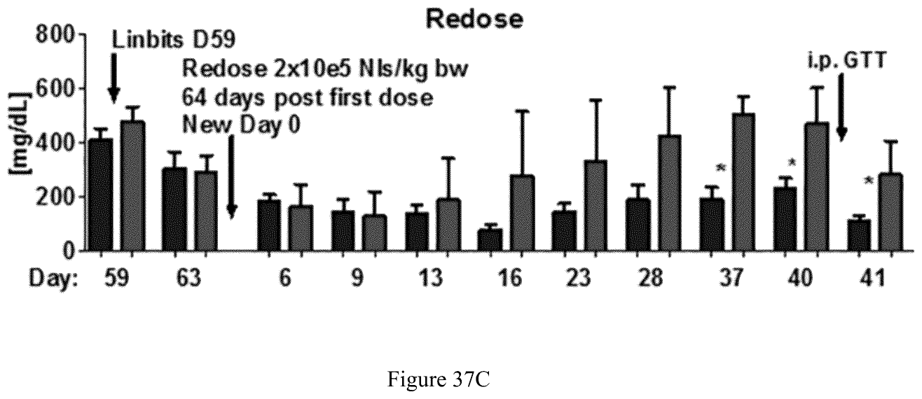

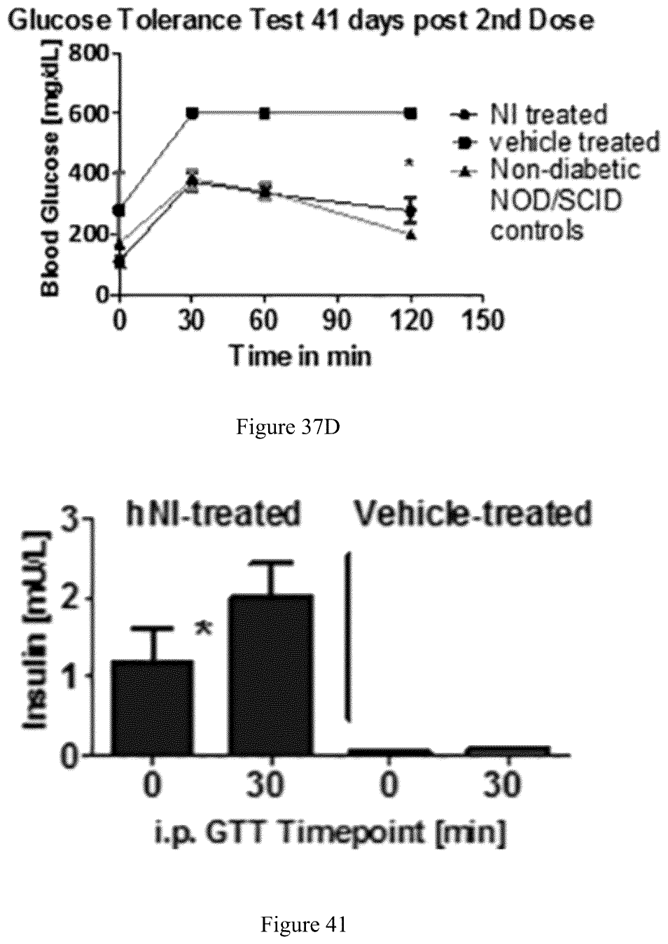

As shown in FIGS. 37 and 38, administration of a single dose of hNIs to diabetic NOD/SCID mice improved glycemic control, as assessed by serum glucose measurements and i.p. GTTs, and this improvement is mediated by the exclusive secretion of human Insulin.

Administration of a second dose of hNIs establishes euglycemia in previously treated dysglycemic mice. While no mice in the treated group died, 4 animals died in the vehicle treated control group, and while hNI therapy significantly improved glycemic control vs. the vehicle group for 7 weeks, normoglycemia was not maintained (FIGS. 37 and 38).

To test whether a second dose of hNIs could achieve euglycemia in the incompletely controlled mice shown in FIGS. 37A and 37B and 38, at day 59 after the first dose, all surviving mice (n=6 in the treatment group; n=2 in the vehicle group) were again treated with insulin pellets (Linbits), and once blood glucose levels were normalized, they were re-dosed i.p. as before with 2×10e5 hNIs per kg bw (blue) or vehicle (red) on day 64 post the initial treatment. A second i.p. GTT was administered on day 41 post the second dose of hNI. After the second dose, blood glucose levels and i.p. GTTs (FIGS. 37D and 37E) were normalized in the hNI treatment group to the pattern observed in non-diabetic NOD/SCID mice, while the i.p. GTT in the vehicle treated group remained abnormal.

Human insulin is detected in serum from hNI- but not vehicle-treated mice. Serum collected during the i.p. GTT depicted in FIG. 37D was assayed by ELISA for the presence of human insulin as described in Methods. Only serum from hNI treated, but not vehicle treated mice contained human insulin (FIG. 37E). As previously reported, murine insulin secretion during the ip GTT in non-diabetic NOD/SCID was physiological (not shown).

### REFERENCES (THE CONTENTS OF THE ENTIRETY OF EACH OF WHICH ARE INCORPORATED HEREIN BY THIS REFERENCE)

1. Hara M, Wang X, Kawamura T, Bindokas V P, Dizon R F, Alcoser S Y, et al. Transgenic mice with green fluorescent protein-labeled pancreatic beta-cells. Am J Physiol Endocrinol Metab. 2003; 284: E177-E183. doi:10.1152/ajpendo.00321.2002
2. Westenfelder C, Gooch A, Hu Z, Ahlstrom J, Zhang P. Durable Control of Autoimmune Diabetes in Mice Achieved by Intraperitoneal Transplantation of “Neo-Islets,” Three-Dimensional Aggregates of Allogeneic Islet and “Mesenchymal Stem Cells”. Stem Cells Transl Med. 2017; 6: 1631-1643. doi:10.1002/sctm.17-0005
3. Gooch A, Zhang P, Hu Z, Loy Son N, Avila N, Fischer J, et al. Interim report on the effective intraperitoneal therapy of insulin-dependent diabetes mellitus in pet dogs using “Neo-Islets,” aggregates of adipose stem and pancreatic islet cells (INAD 012-776). PLoS One. 2019; 14: e0218688. doi:10.1371/journal.pone.0218688
4. Golcbiewska J E, Bachul P J, Wang L, Matosz S, Basto L, Kijek M R, et al. Validation of a New North American Islet Donor Score for Donor Pancreas Selection and Successful Islet Isolation in a Medium-Volume Islet Transplant Center. Cell Transplant. 2019; 28: 185-194. doi:10.1177/0963689718816989
5. Yeh C-C, Wang L-J, Mcgarrigle J J, Wang Y, Liao C-C, Omami M, et al. Effect of Manufacturing Procedures on Human Islet Isolation from Donor Pancreata Standardized by the North American Islet Donor Score. Cell Transplant. 2017; 26: 33-44. doi:10.3727/096368916X692834
6. Burnett D R, Huyett L M, Zisser H C, Doyle F J, Mensh B D. Glucose Sensing in the Peritoneal Space Offers Faster Kinetics Than Sensing in the Subcutaneous Space. Diabetes. 2014; 63: 2498-2505. doi:10.2337/db13-1649
7. King A J F. The use of animal models in diabetes research. Br J Pharmacol. 2012; 166: 877-94. doi:10.1111/j.1476-5381.2012.01911.x
8. DelaRosa O, Sánchez-Correa B, Morgado S, Ramirez C, del Rio B, Menta R, et al. Human Adipose-Derived Stem Cells Impair Natural Killer Cell Function and Exhibit Low Susceptibility to Natural Killer-Mediated Lysis. Stem Cells Dev. 2012; 21: 1333-1343. doi:10.1089/scd.2011.0139
9. Burr S P, Dazzi F, Garden O a. Mesenchymal stromal cells and regulatory T cells: the Yin and Yang of peripheral tolerance? Immunol Cell Biol. 2013; 91: 12-18. doi:10.1038/icb.2012.60
10. English K. Mechanisms of mesenchymal stromal cell immunomodulation. Immunol Cell Biol. 2013; 91: 19-26. doi:10.1038/icb.2012.56
11. Kim Y-H, Wee Y-M, Choi M-Y, Lim D-G, Kim S-C, Han D-J. Interleukin (IL)-10 induced by CD11b(+) cells and IL-10-activated regulatory T cells play a role in immune modulation of mesenchymal stem cells in rat islet allografts. Mol Med. 2011; 17: 697-708. doi:10.2119/molmed.2010.00098
12. Spaggiari G M, Moretta L. Cellular and molecular interactions of mesenchymal stem cells in innate immunity Immunol Cell Biol. 2013; 91: 27-31. doi:10.1038/icb.2012.62
13. LeBlanc K, Davies L C. Mesenchymal stromal cells and the innate immune response Immunol Lett. 2015; 168: 140-146. doi:10.1016/j.imlet.2015.05.004
14. Plock J A, Schnider J T, Zhang W, Schweizer R, Tsuji W, Kostereva N, et al. Adipose- and Bone Marrow-Derived Mesenchymal Stem Cells Prolong Graft Survival in Vascularized Composite Allotransplantation. Transplantation. 2015; 99: 1765-1773. doi:10.1097/TP.0000000000000731
15. Vrabelova D, Adin C A, Kenzig A, Gilor C, Xu F, Buss J L, et al. Evaluation of a high-yield technique for pancreatic islet isolation from deceased canine donors. Domest Anim Endocrinol. 2014; 47: 119-126. doi:10.1016/j.domaniend.2013.01.006
16. Woolcott O O, Bergman R N, Richey J M, Kirkman E L, Harrison L N, Ionut V, et al. Simplified Method to Isolate Highly Pure Canine Pancreatic Islets. Pancreas. 2012; 41: 31-38. doi:10.1097/MPA.0b013e318221fd0e
17. Pittenger M F, Mackay A M, Beck S C, Jaiswal R K, Douglas R, Mosca J D, et al. Multilineage potential of adult human mesenchymal stem cells. Science. 1999; 284: 143-7. Available: http://www.ncbi.nlm.nih.gov/pubmed/10102814
18. Tögel F, Hu Z, Weiss K, Isaac J, Lange C, Westenfelder C. Administered mesenchymal stem cells protect against ischemic acute renal failure through differentiation-independent mechanisms. Am J Physiol Renal Physiol. 2005; 289: F31-42. doi:10.1152/ajprenal.00007.2005
19. Crop M J, Baan C C, Korevaar S S, IJzermans J N M, Alwayn I P J, Weimar W, et al. Donor-Derived Mesenchymal Stem Cells Suppress Alloreactivity of Kidney Transplant Patients. Transplantation. 2009; 87: 896-906. doi:10.1097/TP.0b013e31819b3d72
20. Tögel F E, Westenfelder C. Mesenchymal stem cells: a new therapeutic tool for AKI. Nat Rev Nephrol. 2010; 6: 179-183. doi:10.1038/nrneph.2009.229
21. Tögel F E, Westenfelder C. Kidney protection and regeneration following acute injury: progress through stem cell therapy. Am J Kidney Dis. 2012; 60: 1012-22. doi:10.1053/j.ajkd.2012.08.034
17. Westenfelder C, Gooch A, Hu Z, Ahlstrom J, Zhang P. Durable Control of Autoimmune Diabetes in Mice Achieved by Intraperitoneal Transplantation of “Neo-Islets,” Three-Dimensional Aggregates of Allogeneic Islet and “Mesenchymal Stem Cells”. Stem Cells Transl Med [Internet]. 2017 July; 6(7):1631-43. Available from: http://www.ncbi.nlm.nih.gov/pubmed/28467694
18. Tögel F E, Westenfelder C. Mesenchymal stem cells: a new therapeutic tool for AKI. Nat Rev Nephrol [Internet]. 2010 March [cited 2013 Sep. 3]; 6(3):179-83. Available from: http://www.ncbi.nlm.nih.gov/pubmed/20186233
19. Tögel F E, Westenfelder C. Kidney Protection and Regeneration Following Acute Injury: Progress Through Stem Cell Therapy. Am J Kidney Dis [Internet]. 2012 December [cited 2013 Aug. 10]; 60(6):1012-22. Available from: http://www.ncbi.nlm.nih.gov/pubmed/23036928

1-34. (canceled)

35. A method of making a cell cluster, the method comprising:
expanding pancreatic islet cells for at least five population doublings; and
forming cell clusters comprising:
the expanded pancreatic islet cells; and
mesenchymal stem cells or adipose stem cells.

36. The method according to claim 1, wherein the pancreatic islet cells are primary pancreatic islet cells obtained from an adult human donor.

37. The method according to claim 1, wherein the pancreatic islet cells are classified as research grade before expansion.

38. The method according to claim 2, wherein the adult human donor had a North American Islet Donor Score (NAIDS) of less than 80.

39. The method according to claim 1, wherein the pancreatic islet cells are obtained from more than one donor.

40. The method according to claim 1, wherein the pancreatic islet cells are obtained from a single donor and more than 100 doses of cell clusters sufficient for the treatment of diabetes are formed.

41. The method according to claim 6, wherein a single dose of cell clusters sufficient for the treatment of diabetes comprises at least 7.00E+09 expanded pancreatic islet cells.

42. A method of making a cell cluster, the method comprising:
expanding pancreatic islet cells; and
forming cell clusters comprising:
the expanded pancreatic islet cells; and
mesenchymal stem cells or adipose stem cells;
wherein the pancreatic islet cells are primary pancreatic islet cells obtained from an adult donor; and
wherein the adult donor had a North America Islet Donor Score (NAIDS) of less than 80.

43. The method according to claim 8, wherein expanding the pancreatic islet cells comprises expanding the pancreatic islet cells for at least five population doublings.

44. The method according to claim 8, wherein the pancreatic islet cells are human cells

45. The method according to claim 8, wherein the primary pancreatic islet cells are classified as research grade.

46. The method according to claim 8, wherein the primary pancreatic islet cells are obtained from a single donor and more than 100 doses of cell clusters sufficient for the treatment of diabetes are formed.

47. The method according to claim 12, wherein a single dose of cell clusters sufficient for the treatment of diabetes comprises at least 7.00E+09 expanded pancreatic islet cells.

48. A method of treating diabetes, the method comprising:
administering to a subject suffering from diabetes a therapeutically sufficient number of the cell clusters of obtained by the method of claim 1 and/or claim 12.

49. The method according to claim 14, wherein the therapeutically sufficient number of the cell clusters comprises at least 7.00E+09 expanded pancreatic islet cells.

50. The method according to claim 14, further comprising:
administering to a subject suffering from diabetes two doses of cell clusters.

51. The method according to claim 16, wherein the subject receiving two doses of cell clusters achieves durable euglycemia.

52. The method according to claim 14, wherein the subject is not administered anti-rejection drugs.

53. The method according to claim 14, wherein the cell clusters are not encapsulated or associated with an encapsulation device.
